SEQUENCE LISTING (517 sequences)

SEQ ID NO: 1 - protein, 124 aa, Artificial Sequence
  Description of Artificial Sequence Synthetic polypeptide
QVQLQQSGGGLVRAGGSLRLSCETSGRTFSSYTMGWFRQAPGKEREFVGTIDWWSSSSSYADSVKGRFTISRDNAKNTVYLQMNSLKPEDTAVYYCAASGKYGLAYSRRDYAYWGQGTQVTVSS

SEQ ID NO: 2 - protein, 127 aa, Artificial Sequence
  Description of Artificial Sequence Synthetic polypeptide
QVQLQQSGGSLVQAGGSLRLSCAASGLPFINYAMGWFRQAPGKDREIVAAIDWNGDSTYYAVSVKGRFTISRDNAKNTVTLQMNSLKPEDTAIYYCASHYQPYIRVSATRRFEADYWGQGTQVTVSS

SEQ ID NO: 3 - protein, 127 aa, Artificial Sequence
  Description of Artificial Sequence Synthetic polypeptide
QVQLQQSGGSLVQAGGSLRLSCAASGLPFINYAMGWFRQAPGKDREIVAAIDWNGDSTYYAVSVKGRFTISRDNAKNTVTLQMNSLKPEDTAIYYCAADYQPYIRVSATRRFEADYWGQGTQVTVSS

SEQ ID NO: 4 - protein, 120 aa, Artificial Sequence
  Description of Artificial Sequence Synthetic polypeptide
QVQLQESGGGLVQAGDSLRLSCATSGRTFSRYTMGWFRQTPGKEREFVAAISWSGTYYTDSVKGRFTISVDNAKNTVYLQMNSLKPEDTAVYYCASGSRRLYYSSDIDYWGQGTQVTVSS

SEQ ID NO: 5 - protein, 120 aa, Artificial Sequence
  Description of Artificial Sequence Synthetic polypeptide
QVQLQESGGGLVQAGDSLRLSCATSGRTFSRYTMGWFRQTPGKEREFVAAISWSGTYYTDSVKGRFTISRDNAKNTVYLQMNSLKPEDTAVYYCVVGSRRLYYSSDINYWGQGTQVTVSS

SEQ ID NO: 6 - protein, 126 aa, Artificial Sequence
  Description of Artificial Sequence Synthetic polypeptide
QVQLQESGGGLVQAGESLRLSCAASGFTFSTYTWDWYRQAPGKQREMVARISRDGITNYADSVKGRFTISRDNAKNTVDLQMNSLKPEDTAVYYCAVVKEDNRYWCHADRNLYRNWGQGTQITVSS

SEQ ID NO: 7 - protein, 9 aa, Artificial Sequence
  Description of Artificial Sequence Synthetic peptide
GRTFSSYTM

SEQ ID NO: 8 - protein, 9 aa, Artificial Sequence
  Description of Artificial Sequence Synthetic peptide
GLPFINYAM

SEQ ID NO: 9 - protein, 9 aa, Artificial Sequence
  Description of Artificial Sequence Synthetic peptide
GLPFINYAM

SEQ ID NO: 10 - protein, 9 aa, Artificial Sequence
  Description of Artificial Sequence Synthetic peptide
GRTFSRYTM

SEQ ID NO: 11 - protein, 9 aa, Artificial Sequence
  Description of Artificial Sequence Synthetic peptide
GRTFSRYTM

SEQ ID NO: 12 - protein, 9 aa, Artificial Sequence
  Description of Artificial Sequence Synthetic peptide
GFTFSTYTW

SEQ ID NO: 13 - protein, 8 aa, Artificial Sequence
  Description of Artificial Sequence Synthetic peptide
IDWWSSSS

SEQ ID NO: 14 - protein, 8 aa, Artificial Sequence
  Description of Artificial Sequence Synthetic peptide
IDWNGDST

SEQ ID NO: 15 - protein, 8 aa, Artificial Sequence
  Description of Artificial Sequence Synthetic peptide
IDWNGDST

SEQ ID NO: 16 - protein, 6 aa, Artificial Sequence
  Description of Artificial Sequence Synthetic peptide
ISWSGT

SEQ ID NO: 17 - protein, 6 aa, Artificial Sequence
  Description of Artificial Sequence Synthetic peptide
ISWSGT

SEQ ID NO: 18 - protein, 7 aa, Artificial Sequence
  Description of Artificial Sequence Synthetic peptide
ISRDGIT

SEQ ID NO: 19 - protein, 17 aa, Artificial Sequence
  Description of Artificial Sequence Synthetic peptide
AASGKYGLAYSRRDYAY

SEQ ID NO: 20 - protein, 20 aa, Artificial Sequence
  Description of Artificial Sequence Synthetic peptide
ASHYQPYIRVSATRRFEADY

SEQ ID NO: 21 - protein, 20 aa, Artificial Sequence
  Description of Artificial Sequence Synthetic peptide
AADYQPYIRVSATRRFEADY

SEQ ID NO: 22 - protein, 15 aa, Artificial Sequence
  Description of Artificial Sequence Synthetic peptide
ASGSRRLYYSSDIDY

SEQ ID NO: 23 - protein, 15 aa, Artificial Sequence
  Description of Artificial Sequence Synthetic peptide
VVGSRRLYYSSDINY

SEQ ID NO: 24 - protein, 20 aa, Artificial Sequence
  Description of Artificial Sequence Synthetic peptide
AVVKEDNRYWCHADRNLYRN

SEQ ID NO: 25 - protein, 111 aa, Vibrio parahaemolyticus
MSNNIKHETDYSHDWTVEPNGGVTEVDSKHTPIIPEVGRSVDIENTGRGELTIQYQWGAPFMAGGWKVAKSHVVQRDETYHLQRPDNAFYHQRIVVINNGASRGFCTIYYH

SEQ ID NO: 26 - protein, 438 aa, Vibrio parahaemolyticus
MTNEYVVTMSSLTEFNPNNARKSYLFDNYEVDPNYAFKAMVSFGLSNIPYAGGFLSTLWNIFWPNTPNEPDIENIWEQLRDRIQDLVDESIIDAINGILDSKIKETRDKIQDINETIENFGYAAAKDDYIGLVTHYLIGLEENFKRELDGDEWLGYAILPLLATTVSLQITYMACGLDYKDEFGFTDSDVHKLTRNIDKLYDDVSSYITELAAWADNDSYNNANQDNVYDEVMGARSWCTVHGFEHMLIWQKIKELKKVDVFVHSNLISYSPAVGFPSGNFNYIATGTEDEIPQPLKPNMFGERRNRIVKIESWNSIEIHYYNRVGRLKLTYENGEVVELGKAHKYDEHYQSIELNGAYIKYVDVIANGPEAIDRIVFHFSDDRTFVVGENSGKPSVRLQLEGHFICGMLADQEGSDKVAAFSVAYELFHPDEFGTEK

SEQ ID NO: 27 - protein, 208 aa, White spot syndrome virus
MHMWGVYAAILAGLTLILVVISIVVTNIELNKKLDKKDKDAYPVESEIINLTINGVARGNHFNFVNGTLQTRNYGKVYVAGQGTSDSELVKKKGDIILTSLLGDGDHTLNVNKAESKELELYARVYNNTKRDITVDSVSLSPGLNATGREFSANKFVLYFKPTVLKKNRINTLVFGATFDEDIDDTNRHYLLSMRFSPGNDLFKVGEK

SEQ ID NO: 28 - protein, 204 aa, White spot syndrome virus
MDLSFTLSVVSAILAITAVIAVFIVIFRYHNTVTKTIETHTGNIETNMDENLRIPVTAEVGSGYFKMTDVSFDSDTLGKIKIRNGKSDAQMKEEDADLVITPVEGRALEVTVGQNLTFEGTFKMWNNTSRKINITGMQMVPKINPSKAFVGSSNTSSFTPVSIDEDEVGTFVCGTTFGAPIAATAGGNLFDMYVHVTYSGTETE

SEQ ID NO: 29 - protein, 118 aa, Artificial Sequence
  Description of Artificial Sequence Synthetic polypeptide
QVQLQQSGGGLVQPGGSLRLSCAGSRFTFSTYPMSWVRQAPGKGVEWVSSISVGGGIKNYADSVKGRFTISRDNAKNTMYLQMNGLKPEDTAVYYCAKGGKTSYTREWGQGTQVTVSS

SEQ ID NO: 30 - protein, 129 aa, Artificial Sequence
  Description of Artificial Sequence Synthetic polypeptide
QVQLQESGGGLVQAGDSLRLSCAASGRTFSRYAMGWFRQAPGKEREFVAAVDWSGGSTAYADSVKGRFTISRDNAKNTVYLQMNSLKPDDTAVYYCAARARDVYGRAWYVEDSSTYDYWGQGTQVTVSS

SEQ ID NO: 31 - protein, 117 aa, Artificial Sequence
  Description of Artificial Sequence Synthetic polypeptide
QVQLQESGGGLVQAGGSLRLSCAASGSMFSINAMGWYRQAPGNEREWVATISRGGITYYDDSVKGRFTISRDNAKNTVYLQMNSLKPEDTAVYFCNAENRRLGDDFWGQGTQVTVSS

SEQ ID NO: 32 - protein, 123 aa, Artificial Sequence
  Description of Artificial Sequence Synthetic polypeptide
QVQLQESGGGLVQAGGSLRLSCAASGRTFSSYTMGWFRQVPGKEREFVAAIRWSGGSRIYADSVKGRFTISRDNTKNVVYLQMTSLKPEDTAVYYCAADRDGYSSYAHQYDYWGQGTQVTVSS

SEQ ID NO: 33 - protein, 117 aa, Artificial Sequence
  Description of Artificial Sequence Synthetic polypeptide
QVQLQESGGGLVQPGGALRLSCAASGSFFSIYAMAWYRQAPGKQRELVAGITSGSETNYADSVKGRFTISRDNAKNTVYLQMNSLKLEDTAVYYCNRWAPLTRLDYWGQGTQVTVSS

SEQ ID NO: 34 - protein, 130 aa, Artificial Sequence
  Description of Artificial Sequence Synthetic polypeptide
QVQLQESGGGLVQAGDSLRLSCAASGRTSSSFAMGWFRQAPGKEREFVGGITRTGGRTYYVDSVKGRFTISRDNAKNTMSLQMNSLKPEDTAVYYCAARWATATSNSIRVYYNEGQYDYWGQGTQVTVSS

SEQ ID NO: 35 - protein, 125 aa, Artificial Sequence
  Description of Artificial Sequence Synthetic polypeptide
QVQLQESGGGLVQAGGSLRLSCAASGGTFSRLTMGWFRQAPGEEREFVAAVSWVAETTDYADSVKGRFSISRDNSKNTVYLQMNSLKPVDTAVYYCAAGPRDMIRSRNIRSYDSWGQGTQVTVSS

SEQ ID NO: 36 - protein, 117 aa, Artificial Sequence
  Description of Artificial Sequence Synthetic polypeptide
QVQLQESGGGLVQAGGSLRLSCAASGSMFNINAMGWYRQAPGKQREWVATISSGGITYYDDSVKGRFTISRDNAKNTVYLQMNSLKPEDTAVYFCNAENRRLGDDYWGQGTQVTVSS

SEQ ID NO: 37 - protein, 117 aa, Artificial Sequence
  Description of Artificial Sequence Synthetic polypeptide
QVQLQESGGGLVQAGGSLRLSCAASGSIFSGNVMGWYRQVPGKLREWVATISSGGITYYDDSVKGRFTISRDNAKNTVYLQMNSLKPEDTAVYFCNLENRRLGDDYWGQGTQVTVSS

SEQ ID NO: 38 - protein, 117 aa, Artificial Sequence
  Description of Artificial Sequence Synthetic polypeptide
QVQLQESGGGLVQAGGSLRLSCAASGSIFSRDAMGWYRQAPGNLREWVATISSGGITYYDDSVKGRFTISRDNAKNTVYLQMNSVKPEDTAVYFCNAENRRLGDDYWGQGTQVTVSS

SEQ ID NO: 39 - protein, 125 aa, Artificial Sequence
  Description of Artificial Sequence Synthetic polypeptide
QVQLQQSGGGLVQAGGSLRLSCAASGGTFSRLTMGWFRQAPGEEREFVAAVSWVAETTDYADSVKGRFSISRDNSKNTVYLQMNSLKPVDTAVYYCAAGPRDMIRSRNIKSYDSWGQGTQVTVSS

SEQ ID NO: 40 - protein, 120 aa, Artificial Sequence
  Description of Artificial Sequence Synthetic polypeptide
QVQLQESGGGLVQAGGSLRLSCVASGTIFSINKMGWYRQAPEKERELVAVARSGGIINYADSVKGRFTISRDDAKNTVYLQMNSLRPDDTAVYFCNALIHTRYDRVTGYWGQGTQVTVSS

SEQ ID NO: 41 - protein, 117 aa, Artificial Sequence
  Description of Artificial Sequence Synthetic polypeptide
QVQLQESGGGLVQAGGSLRLSCAASGSIFSINAMGWYRQAPGKLREWVATISSGGITYYDDSVKGRFTISRDNAKNTVYLQMNSLKPEDTAVYFCNAENPRLGDDYWGQGTQVTVSS

SEQ ID NO: 42 - protein, 120 aa, Artificial Sequence
  Description of Artificial Sequence Synthetic polypeptide
QVQLQQSGGGLVQAGGSLRLSCAASGSSFRSNAIGWYRQFPGKSRELIAVITRSGSTQYADSVKGRFTASRDNAKNMIYLQMNNLKLEDTAVYYCHDETMKLISVKNDYWGQGSQVTVSS

SEQ ID NO: 43 - protein, 117 aa, Artificial Sequence
  Description of Artificial Sequence Synthetic polypeptide
QVQLQESGGGLVQAGGSLRLSCAASGSMFSRNAMGWYRQAPGKQREWVATISSGGITYYDDSVKGRFTISRDNAKNTVYLQMNSLKPEDTAVYFCNAENRRLGDDYWGQGTQVTVSS

SEQ ID NO: 44 - protein, 126 aa, Artificial Sequence
  Description of Artificial Sequence Synthetic polypeptide
QVQLQESGGGLVQAGGSLRLSCATSGLTFSSYAMGWFRQAPGKEREFVATISWSGKSTRYSDSVKGRFTISRDNAKNTVYLQMNSLKPEDTAVYYCAADYQRLGLLRVGVAEYDYWGQGIQVTVSS

SEQ ID NO: 45 - protein, 125 aa, Artificial Sequence
  Description of Artificial Sequence Synthetic polypeptide
QVQLQQSGGGLVQAGGSLRLSCQASGRSGSTSFMGWFRQAPGKEREFVAAIRWSSGMTYYADSVKGRFTISRDNAKNTVDLQMNSLKPEDTAVYYCAADNYPLHIGHQDHEVDYWGQGTQVTVSS

SEQ ID NO: 46 - protein, 121 aa, Artificial Sequence
  Description of Artificial Sequence Synthetic polypeptide
QVQLQESGGGLVQPGGSLRLSCAASGSIFSFNAMGWYRQAPGKQRELVAAITKGGSTSYADSVKGRFTISVDNAKNTVYLQMNSLTPEDTAVYYCNVKTLRSALFPGYEYWGQGTQVTVSS

SEQ ID NO: 47 - protein, 125 aa, Artificial Sequence
  Description of Artificial Sequence Synthetic polypeptide
QVQLQQSGGGLVQAGGSLRLFCAASGGTFSRLTMGWFRQAPGEEREFVAAVSWVAETTDYADSVKGRFSISRDNSKNTVYLQMNNLKPVDTAVYYCAAGPRDMIRSRNIRSYDSWGQGTQVTVSS

SEQ ID NO: 48 - protein, 125 aa, Artificial Sequence
  Description of Artificial Sequence Synthetic polypeptide
QVQLQESGGGLVQAGGSLRLSCAASGGTFSRLTLGWFRQAPGEEREFVAAVSWVAEMTDYADSVKGRFSISRDNSKNTVYLQMNSLKPVDTAVYYCAAGPRDMVRSRNIRSYDSWGQGTQVTVSS

SEQ ID NO: 49 - protein, 123 aa, Artificial Sequence
  Description of Artificial Sequence Synthetic polypeptide
QVQLQESGGGLVQAGGSLRLSCAASGRTFSTYSMGWFRQVPGKEREFVAAIRWSGGSRTYADSVKGRFTISRDNTKNVVYLQMTSLKPEDTAVYYCAADRDGYSSYAHQYDYWGQGTQVTVSS

SEQ ID NO: 50 - protein, 117 aa, Artificial Sequence
  Description of Artificial Sequence Synthetic polypeptide
QVQLQESGGGLVQAGGSLRLSCAASGRLFNINAMGWYRQAPGKQREWVATISSGGITYYDDSVKGRFTISRDNAKNTVYLQMDSLKPGDTAVYFCNAENRGLGDDYWGQGTQVTVSS

SEQ ID NO: 51 - protein, 128 aa, Artificial Sequence
  Description of Artificial Sequence Synthetic polypeptide
QVQLQESGGGWVQTGGSLRLSCAASGRTLSNYAMGWFRQAPGKEREFVAAISRSGMSTDAPNSVKGRFTVSRDNAKNTMYLHLNSLKPEDTAVYYCAARGGLPNPSRTYGFEEQYDYWGQGTQVTVSS

SEQ ID NO: 52 - protein, 127 aa, Artificial Sequence
  Description of Artificial Sequence Synthetic polypeptide
QVQLQESGGGLVQAGGSLRLSCAASGRTVSSLPMGWFRQAPGKEREFVAALNWSGTSTYYEDSVKGRFTISRDNAKNTLYLQMNNLKPEDTAVYYCAAKGAIYYSYSPRNNNNYDVWGQGTQVTVSS

SEQ ID NO: 53 - protein, 116 aa, Artificial Sequence
  Description of Artificial Sequence Synthetic polypeptide
QVQLQESGGGLVQAGGSLRLSCAASGSMFNINAMGWYRQAPGKQREWVATISSGGITYYDDSVKGRFTISRDNAKNTVHLQMNSLKPEDTAVYFCNAENRLGDDYWGQGTQVTVSS

SEQ ID NO: 54 - protein, 118 aa, Artificial Sequence
  Description of Artificial Sequence Synthetic polypeptide
QVQLQQSGGGLVQAGGSLRLACAVSETTLATNAMAWYRQAQGKRREWVATISSVSSGGITNYSGSVKGRFTISRDNAKNTVFLQMNSLQPEDTAVYYCNGVRRGRSYWGQGTQVTVSS

SEQ ID NO: 55 - protein, 120 aa, Artificial Sequence
  Description of Artificial Sequence Synthetic polypeptide
QVQLQQSGGGLVQAGGSLRLSCAASGSSFRSNAIGWYRQSPGKSRELIAVITRSGSTQYADSVKGRFTASRDNAKNMIYLQMNSLKPEDTAVYYCHDETMKLITGKNDYWGQGTQVTVSS

SEQ ID NO: 56 - protein, 123 aa, Artificial Sequence
  Description of Artificial Sequence Synthetic polypeptide
QVQLQQSGGGLVQVGGSLRLSCAASGRTFSSYAMGWFRQVPGKEREFVAAIKWSGGSRTYADSVAGRFTISRDNTKNVVYLQMTSLKPEDTAVYYCAADRDGYSRYAHQYDYWGQGTQVTVSS

SEQ ID NO: 57 - protein, 125 aa, Artificial Sequence
  Description of Artificial Sequence Synthetic polypeptide
QVQLQQSGGGLVQAGGSLRLSCAASGGTFSRLTIGWFRQAPGEERVFVAAVSWVAETTDYADSVKGRFSISRDNSKNTVYLQMNSLKPVDTAVYYCAAGPRDMIRSRNIRSYDSWGQGTQVTVSS

SEQ ID NO: 58 - protein, 116 aa, Artificial Sequence
  Description of Artificial Sequence Synthetic polypeptide
QVQLQQSGGGLVPGGGSLRLSCAASGVTFSDYPMAWYRTAPGKQRELVASISAGGGLIKYVDSVKGRFTISRDNAKNTLYLQMNSLKPEDTGVYLCNLKTSYFWPWGQGTQVTVSS

SEQ ID NO: 59 - protein, 125 aa, Artificial Sequence
  Description of Artificial Sequence Synthetic polypeptide
QVQLQESGGGLVQAGDSLRLSCKASGGTFSRLTIAWFRQAPGKEREFVTAVSWVAQTTDYADSVKGRFSISRDNSKNTVYLQMNSLKPLDTAVYYCAAGPQDMIRSRNIRSYISWGQGTQVTVSS

SEQ ID NO: 60 - protein, 125 aa, Artificial Sequence
  Description of Artificial Sequence Synthetic polypeptide
QVQLQQSGGGLVQAGGSLRLSCAASGGTFSRLTMGWFRQAPGEEREFVAAVSWVAGTTDYADSVKGRFTISRDNSKNTVHLQMNSLKPVDTAVYYCAAGPRDMIRSRNIRSYDSWGQGTQVTVSS

SEQ ID NO: 61 - protein, 114 aa, Artificial Sequence
  Description of Artificial Sequence Synthetic polypeptide
QVQLQESGGGLVQPGGSLRLSCTASGTIFRSKSMAWYRQAPGQGRETVAHISGLGHTNYVESVKGRFTVSRDDAKNAVYLQMSSLKPEDTAVYYCNTFTAAFSWGQGTQVTVSS

SEQ ID NO: 62 - protein, 125 aa, Artificial Sequence
  Description of Artificial Sequence Synthetic polypeptide
QVQLQQSGGGLVQAGGSLRLSCAASGGSFSRLTLGWFRQAPGEEREFVAAVSWVAETTDYADSVKGRFSISRDNSKNTVYLQMNSLKPVDTAVYYCAAGPRDMIRSRNIRSYASWGQGTQVTVSS

SEQ ID NO: 63 - protein, 126 aa, Artificial Sequence
  Description of Artificial Sequence Synthetic polypeptide
QVQLQESGGGLVQAGGSLRLSCATSGLTFSNYAMGWFRQAAGKEREFVATISWSGKSTRYADSVKGRFTISRDNAKNTVDLRMNSLKPEDTAVYYCAAEYQRLGLLRDGVADYSYWGQGTQVTVSS

SEQ ID NO: 64 - protein, 125 aa, Artificial Sequence
  Description of Artificial Sequence Synthetic polypeptide
QVQLQESGGGLVQAGGSLRLSCAASGGTFSRLTMGWFRQAPGEEREFVAAVSWVAETTDYADSVKGRFSISRDNSKNTVYLQMNSLKPVDTAVYYCAAGPRDMIRSRNIRSYVSWGQGTQVTVSS

SEQ ID NO: 65 - protein, 116 aa, Artificial Sequence
  Description of Artificial Sequence Synthetic polypeptide
QVQLQESGGGLVQAGNSLKLSCVASGRTFSSYPMGWYRTAPGKQRELVASISAGGGLIKYVDSVKGRFTISRDNAKNTLYLQMNSLKPEDTGVYLCNLKTSYFWPWGQGTQVTVSS

SEQ ID NO: 66 - protein, 115 aa, Artificial Sequence
  Description of Artificial Sequence Synthetic polypeptide
QVQLQESGGGLVQAGGSLRLSCAASGSIFSGNVMGWYRQVPGKQRDLVATITGGGITRYADSVKGRFTISRDNAKNTVYLQMNSLKPEDTAVYYCNYRRIMQQYWGKGTLVTVSS

SEQ ID NO: 67 - protein, 115 aa, Artificial Sequence
  Description of Artificial Sequence Synthetic polypeptide
QVQLQESGGGLVQAGGSLRLSCAASGIVFSSIVMAWYRQAPGKQRELVASITNGGLVNSGDSVKGRFTISRDNAKNTVYLQMNSLKPEDTAVYYCNARRIMTSYWGQGTQVTVSS

SEQ ID NO: 68 - protein, 115 aa, Artificial Sequence
  Description of Artificial Sequence Synthetic polypeptide
QVQLQESGGGLVQPGGSLRLSCAASGSIFSGNVMGWYRQVPGKQRDLVATITGGGITRYADSVKGRFTISRDNAKNTVYLQMNSLKPEDTAVYYCHYRRIMQQYWGQGTQVTVSS

SEQ ID NO: 69 - protein, 115 aa, Artificial Sequence
  Description of Artificial Sequence Synthetic polypeptide
QVQLQESGGGLVQAGGSLRLSCAASGSISNSYVMGWYRQAPGKQRELVATITSGGLTNYAQSLKGRFTISRDNAKNTVYLQMTSLEPEDTAVYYCNARVIFTTYWGQGTQVTVST

SEQ ID NO: 70 - protein, 125 aa, Artificial Sequence
  Description of Artificial Sequence Synthetic polypeptide
QVQLQQSGGGLVQAGGSLRLSCVASTVTFSRYAMGWFRQAPGKEREVVAGISGSGHRTYYGDFVKGRFTISRDNAKKTVYLQMNNLKPEDTAVYYCAGDLVAKFDSAYRVSYDSWGQGTQVTVSS

SEQ ID NO: 71 - protein, 115 aa, Artificial Sequence
  Description of Artificial Sequence Synthetic polypeptide
QVQLQESGGGLVQAGGSLRLSCEASGSIFSGNVMGWYRQVPGKQRDLVATMTGGGVTRYADSVKARFTISRDNAKNTVYLQMNSLKPEDTGVYYCHYRRIMQQYWGQGTQVTVSS

SEQ ID NO: 72 - protein, 115 aa, Artificial Sequence
  Description of Artificial Sequence Synthetic polypeptide
QVQLQESGGGLVQAGGSLRLSCAASGSIFSGNVMGWYRQVPGKQRDLVATITGGGITRYADSVKGRFTISRDNAKNKVYLQMNGLKPEDTAVYFCFYRRIMQQYWGQGTQVTVSS

SEQ ID NO: 73 - protein, 115 aa, Artificial Sequence
  Description of Artificial Sequence Synthetic polypeptide
QVQLQESGGGLVQAGGSLRLSCAASGSIFSGNVMGWYRQVPGKQRDLVATITGGGITHYADSVKGRFTISRDNAKNTVYLQMNSLKPEDTAVYYCLYRRIMQQYWGQGTQVTVSS

SEQ ID NO: 74 - protein, 115 aa, Artificial Sequence
  Description of Artificial Sequence Synthetic polypeptide
QVQLQESGGGLVQAGGSLRLSCAASGSIFSGNVMGWYRQVPGNQRELVATITGGGVTRYADSVKARFTISRDNAKNTVYLQMNSLKPEDTAVYYCHYRRIMQQSWGQGTQVTVSS

SEQ ID NO: 75 - protein, 115 aa, Artificial Sequence
  Description of Artificial Sequence Synthetic polypeptide
QVQLQESGGGSVPPGGSLRLSCAASGSIFSGNVMAWYRQVPGKQRDLVASMTGGGVTRYADSVIARFTISRDNVKNTVYLQMNSLKPEDTAVYYCHYRRIMQQYWGQGTQVTVSS

SEQ ID NO: 76 - protein, 115 aa, Artificial Sequence
  Description of Artificial Sequence Synthetic polypeptide
QVQLQESGGGLVQAGGSLRLSCEASGIIFSSNVMGWYRQAPGKQRELVASRTSGGLTNYADSAKGRFTISRDNAKNTVYLQMNSLKPEDTAVYYCNARRLFTNYWGQGTQVTVSS

SEQ ID NO: 77 - protein, 115 aa, Artificial Sequence
  Description of Artificial Sequence Synthetic polypeptide
QVQLQQSGGGLVQPGGSLTLSCAASGSIASGNVLGWYRQAPGKQRELVATITSGGLTHYKDSVKGRFTISRDNAKNMVFLQMNSLKPEDTAVYYCNYRRLATGYWGQGTQVTVSS

SEQ ID NO: 78 - protein, 115 aa, Artificial Sequence
  Description of Artificial Sequence Synthetic polypeptide
QVQLQESGGGLVQAGGSLRLSCEASGSIFSGNVLGWYRQVPGKQRDLVATITGGGITRYADSVKGRFTISRDNAKNTVYLQMNALKPEDTAVYYCHYRRIMQQYWGQGTQVTVSS

SEQ ID NO: 79 - protein, 119 aa, Artificial Sequence
  Description of Artificial Sequence Synthetic polypeptide
QVQLQESGGGLVQAGGSLRLSCRASGRTFSSYPMGWFRQAPGKEREQIAGISRSGDPGKYAASVSGRFTISRDNAKNTLYLQMNSLKPEDTAVYYCAARQIYSNNYSYWGQGTQVTVSS

SEQ ID NO: 80 - protein, 119 aa, Artificial Sequence
  Description of Artificial Sequence Synthetic polypeptide
QVQLQESGGGLVQAGGSLRLSCAASGRTFSSYPMGWFRQAPGSEREQIAGISRSGVPGKYADSVSGRFTISRDNAKNTLYLQMNSLKPEDTAVYYCAARSIYSNNYSYWGQGTQVTVSS

SEQ ID NO: 81 - protein, 119 aa, Artificial Sequence
  Description of Artificial Sequence Synthetic polypeptide
QVQLQQSGGGLVQAGGSLRLSCAASGRTFSSYPMGWFRQAPGSEREQIAGISRSGVSGKYADSVSGRFTISRDNAKNTLYLQMNSLKPEDTAVYYCAARSIYSNNYSYWGQGTQVTVSS

SEQ ID NO: 82 - protein, 119 aa, Artificial Sequence
  Description of Artificial Sequence Synthetic polypeptide
QVQLQQSGGGLVQAGGSLRLSCTASGRTFSSYPMGWFRQAPGKEREQIAGISRSGNPGKYADSVSGRFTISRDNAKNTLYLQMNSLKPEDTAVYYCAARQIYSNNYSYWGQGTQVTVSS

SEQ ID NO: 83 - protein, 119 aa, Artificial Sequence
  Description of Artificial Sequence Synthetic polypeptide
QVQLQESGGGLVQTGDSLRLACAASGRTFSSYPMAWYRQAPGQEREFVAGINRNGNIPVYADSVKGRFTISRDNAKNTVYLQMNNLKPEDTAVYYCAARTIYDSHYTSWGQGTQVTVSS

SEQ ID NO: 84 - protein, 130 aa, Artificial Sequence
  Description of Artificial Sequence Synthetic polypeptide
QVQLQQSGPGLVKPSETLSLTCTVSDGAITGSYYVWSWIRQPPGKGLEWMGVITYDGSTYYNPSLESRTSISRDTSKNQFSLQLDSVTREDTAVYYCARAGEEYICSGYGCHGSLGLDYWGKGTLVTVSS

SEQ ID NO: 85 - protein, 116 aa, Artificial Sequence
  Description of Artificial Sequence Synthetic polypeptide
QVQLQESGGGLVQPGGSLGLSCAASGFTFGSYAMSWVRQAPGKGPEWVSGINSGDGRITYADSVKGRFTISRDNTKNTLYLQMNSLKPEDTAVYYCATGIRTPIIWGQGTQVTVSS

SEQ ID NO: 86 - protein, 110 aa, Artificial Sequence
  Description of Artificial Sequence Synthetic polypeptide
QVQLQESGGGLVQAGGSLRLSCAASGSIFTSDVGWNRQAPGSVREVVARMTSAGTTIYGDDVMGRFTISRDNAKSTVYLQMNSLLPEDTGVYYCGVGRFWGQGTQVTVSS

SEQ ID NO: 87 - protein, 119 aa, Artificial Sequence
  Description of Artificial Sequence Synthetic polypeptide
QVQLQESGGGLVQAGGSLRLSCAASGRTFSSYPMGWFRQAPGKEREQIAGISRSGDPGKYAASVSGRFTISRDNAKNTLYLQMNSLKPEDTAVYYCAAREIYSNNYSYWGQGTQVTVSS

SEQ ID NO: 88 - protein, 119 aa, Artificial Sequence
  Description of Artificial Sequence Synthetic polypeptide
QVQLQESGGGLVQAGGSLRLSCAASGRTFSSYAMGWFRRAPGKEREQIAGISRSGNPGKYADSVSGRFTISRDNAKNTLYLQMNSLKPEDTAVYYCAARQIYSNNYSYWGQGTQVTVSS

SEQ ID NO: 89 - protein, 111 aa, Artificial Sequence
  Description of Artificial Sequence Synthetic polypeptide
QVQLQESGGGLVQAGGSLRLSCVVSGMLFSIRNMRWYRQAPGKQRELVAQIGSSGNTDYVESVKGRFTISRDNAKNTVYLQMNSLKPEDTAVYFCNALNYWGKGTLVTVSS

SEQ ID NO: 90 - protein, 126 aa, Artificial Sequence
  Description of Artificial Sequence Synthetic polypeptide
QVQLQQSGGDLVQAGGSLRLSCAASMRTFNSRTIGWFRQAPGKGRELAAAIAWTGGNTYYADSVKGRFTISRDNAKNTVYLQMNSLKPEDTAVYYCAAQTRPYDLPSIRPDDYASRGQGTQVTVSS

SEQ ID NO: 91 - protein, 126 aa, Artificial Sequence
  Description of Artificial Sequence Synthetic polypeptide
QVQLQESGGGLVQAGGSLRLSCAASGRTFSSYAMGWFRQSPGKDREFVAAVSWSGGSTYYADSLKGRFTISRDNAKNTVYLQMNSLKPEDTADYYCAAQRVMDYYRPRTESAYAYWGQGTRVTVSS

SEQ ID NO: 92 - protein, 127 aa, Artificial Sequence
  Description of Artificial Sequence Synthetic polypeptide
QVQLQESGGGLVQAGGSLRLSCAASEYIFSNFGMGWFRQAPGKEREFVGAISRSGSRMSYADSVKGRFIISRDNTKNTVYLQMNSLKPEDTAVYYCAAVYGQYSYHYSSDSKQYSYWGQGTQVTVSS

SEQ ID NO: 93 - protein, 128 aa, Artificial Sequence
  Description of Artificial Sequence Synthetic polypeptide
QVQLQESGGGLVQAGGSLRLSCGASGGTNSNYAMGWFRQPPGKKREFVAALSWSGYNTHYADSVKGRFTISRPSARTVDLQMNNVKPEDTAVYYCAARLSGRTAGSRTYYAEGQYDYRGQGTQVTVSS

SEQ ID NO: 94 - protein, 122 aa, Artificial Sequence
  Description of Artificial Sequence Synthetic polypeptide
QVQLQQSGGGLVQAGGSLRLSCAASGRTSSSSYLGWFRQAPGKEREFVASIRWSDGSTYYRDSVEGRFTISRDNAKNTVYLRMNSLKPEDTAVYYCAAATTDWGPRGPYNYWGQGTLVTVSS

SEQ ID NO: 95 - protein, 122 aa, Artificial Sequence
  Description of Artificial Sequence Synthetic polypeptide
QVQLQQSGGREVRPGDSLRLSCRASGRTSGAWNMAWFRQAPGKDREFVAAISGSGRTTEYADSAKGRFTISRDMAKNTVYLQMNSLKPEDTAVYNCAASTFDWGPRGPYRLWGQGTQVTVSS

SEQ ID NO: 96 - protein, 127 aa, Artificial Sequence
  Description of Artificial Sequence Synthetic polypeptide
QVQLQESGGGLVQTGGSLRLSCAASGRTFSNYVIGWFRQAPGKEREFVAAVGRGINSAYHATHYSESVKDRFTTSRDNAKNTGFLQMNSLKTEDTAVYYCAVTSRWGQFDRTDFNSWGQGTQVTVSS

SEQ ID NO: 97 - protein, 111 aa, Artificial Sequence
  Description of Artificial Sequence Synthetic polypeptide
QVQLQESGGGLVQAGGSLRLSCVVSGMLFSIRNMRWYRQAPGKQRELVAQIGTSGATDYVGSVEGRFTISRDNPKNTVYLQMNSLKPEDTAVYFCNALNYWGEGTLVTVSS

SEQ ID NO: 98 - protein, 122 aa, Artificial Sequence
  Description of Artificial Sequence Synthetic polypeptide
QVQLQESGGGLVRPGDSLTLSCTYSGQTFTNSGMAWFRQRPGKEREFVAAVSRSGLGRRYADSVRGRFTITRDNGKNTANLQMDSLKPEDTAVYSCAATTLDWGPRGPYRYWGQGTQVTVSS

SEQ ID NO: 99 - protein, 121 aa, Artificial Sequence
  Description of Artificial Sequence Synthetic polypeptide
QVQLQQSGGGLVQTGGSLRLSCAASGSIFSIDFMGWYRQAPGNPREFVARIRGGNTYYADSVKGRFNISRDNAENTVYMQMNSLKSEDTAVYYCNAQITMRGGTWSTSEYWGQGTQVNVSS

SEQ ID NO: 100 - protein, 131 aa, Artificial Sequence
  Description of Artificial Sequence Synthetic polypeptide
QVQLQESGGGLVQAGGSLRLTCAASGRTLSSYLSSYAMGWFRQAPGKERESVATITWNGDRTLYADAVKGRFTISRDNAKNTVYLQMNSVIPEDTAVYYCAADTVGRWRSTLSVRDEYDYWGQGTQVTVAS

SEQ ID NO: 101 - protein, 124 aa, Artificial Sequence
  Description of Artificial Sequence Synthetic polypeptide
QVQLQESGGGVVETGGSLSVSCVASGRTFSAYTMAWFRQSPGKEREFVASMSRGSAAYYTDSVRGRFAISRVGDKNTVHLQMRDLKPEDTAVYYCAGGSPGSSQIATPEAYTYWGQGTQVTVSA

SEQ ID NO: 102 - protein, 115 aa, Artificial Sequence
  Description of Artificial Sequence Synthetic polypeptide
QVQLQESGGGLVQAGGSLRLSCAASGSISSSHVMGWYRQAPGKQRELVATITSGGSTIYADSVKGRFTVSRDNAKNTVYLQMNSLKSEDTAVYYCHARRLWNTYWGQGTQVTVSS

SEQ ID NO: 103 - protein, 115 aa, Artificial Sequence
  Description of Artificial Sequence Synthetic polypeptide
QVQLQESGGGLVQAGGSLRLSCAASGSISSSFVMGWYRQAPGKQRELVATITSGGSTIYADSVKGRFTVSRDNAKNTVYLQMNSLKSEDTAVYYCHARRLWNTYWGQGTQVTVSS

SEQ ID NO: 104 - protein, 116 aa, Artificial Sequence
  Description of Artificial Sequence Synthetic polypeptide
QVQLQESGGGAVQAGGSLRLSCAGPRSIFSGNAMAWYRQVPGKQRETVATVNTGGLTWYGDFVKGRFTISRDDAKNTLLLQMDSLKPEDTAVYYCNAVLVRARGMWGQGTQVTVSS

SEQ ID NO: 105 - protein, 115 aa, Artificial Sequence
  Description of Artificial Sequence Synthetic polypeptide
QVQLQESGGGSVQPGGSLRLSCSASGDRLSSYVMGWYRQAPGKQRELVATVTSGGRTNYADSVKGRFTISRDNAKNTVYLQMNSLKPEDTAVYYCNARILFTNYWGQGTQVTVSS

SEQ ID NO: 106 - protein, 115 aa, Artificial Sequence
  Description of Artificial Sequence Synthetic polypeptide
QVQLQESGGGLVQPGGSLQLSCVASGSVLSRYVMGWYRQAPGKQRELVATITSGGITRYADSMKGRFTISRDNAKNTVHLQMSSLKPEDTAVYYCNARALWNTYWGQGTQVTVSS

SEQ ID NO: 107 - protein, 115 aa, Artificial Sequence
  Description of Artificial Sequence Synthetic polypeptide
QVQLQESGGGLVQAGGSLRLSCSASGDAFSRYVMGWYRQAPGKQRELVATITSGGSTIYADSVKGRFTVSRDNAKNTVYLQMNSLKSEDTAVYYCHARRLWNTYWGQGTQVTVSS

SEQ ID NO: 108 - protein, 115 aa, Artificial Sequence
  Description of Artificial Sequence Synthetic polypeptide
QVQLQQSGGGLVQAGGSLRLSCAASGSISSSYVMGWYRQAPGKQRELVATITSGGSTIYADSVKGRFTVSRDNAKNTVYLQMNSLKSEDTAVYYCHARRLWNTYWGQGTQVTVSS

SEQ ID NO: 109 - protein, 115 aa, Artificial Sequence
  Description of Artificial Sequence Synthetic polypeptide
QVQLQQSGGGLVQAGGSLRLSCSASGSISSSHVMGWYRQAPGKQRELVATITSGGSTIYADSVKGRFTVSRDNAKNTVYLQMNSLKSEDTAVYYCHARRLWDTYWGQGTQVTVSS

SEQ ID NO: 110 - protein, 115 aa, Artificial Sequence
  Description of Artificial Sequence Synthetic polypeptide
QVQLQESGGGSVQAGGSLRLSCAASESMFRDHNMGWYRQAPGKQRELVATISRGGLINYGDSVRGRFTISRDNAKNTIYLQMNSLKVEDTAVYYCNARRLLTTVWGQGTQVTVSP

SEQ ID NO: 111 - protein, 116 aa, Artificial Sequence
  Description of Artificial Sequence Synthetic polypeptide
QVQLQESGGGLVQPGGSLRLSCSASGNRFSSSYVMGWYRQAPGKQRELVATVTSGGLTHFKDSVKGRFTISRDNAKNTVYLQMNSLKPEDTAVYYCNARILLTNYWGQGTQVTISS

SEQ ID NO: 112 - protein, 115 aa, Artificial Sequence
  Description of Artificial Sequence Synthetic polypeptide
QVQLQQSGGGLVQPGGSLRLSCSASGIRLGSYVMGYYRQAPGKQRELVATVTSGGTTNRADSVKGRFTISRDNAKNAVYLQMNSLKPEDTAVYYCNARILFTNYWGQGTQVTVSS

SEQ ID NO: 113 - protein, 115 aa, Artificial Sequence
  Description of Artificial Sequence Synthetic polypeptide
QVQLQESGGGLVQPGGSLRLSCAASGIVFANHVMGWYRQAPGKQRELVASITNGGLINSVDSVAGRFTISRDNAKNTVYLQMNNLKPEDTAVYYCNARRLYQQYWGQGTQVTVSS

SEQ ID NO: 114 - protein, 125 aa, Artificial Sequence
  Description of Artificial Sequence Synthetic polypeptide
QVQLQESGGGLVQAGGSLRLSCRVSGRTVGSYAMGWFRLQPGKERQFVAAIGWSGASTLYAESVKGRFTISRDNAKNTVYLQMNSLKPEDTAVYYCAQRPSSRYASRYLGDYAYWGQGTQVTVSS

SEQ ID NO: 115 - protein, 115 aa, Artificial Sequence
  Description of Artificial Sequence Synthetic polypeptide
QVQLQESGGGLVQAGGSLRLSCAASGSIGSDYVLGWYRQAPGKQRELVATITSGGLTHYGDSVKGRFTISRDNAKNTVYVQMNSLKFEDTAIYYCNARRLFRNYWGQGTQVTVSS

SEQ ID NO: 116 - protein, 115 aa, Artificial Sequence
  Description of Artificial Sequence Synthetic polypeptide
QVQLQQSGGGLVQAGGSLRLSCAASGSIRSSNVMGWYRQTPGKQRELVATMTAGGLTNYADSVKGRFTISRDNAKNTVYLQMNSLKPEDTAVYYCHYRRIFNVYWGQGTQVTVSS

SEQ ID NO: 117 - protein, 118 aa, Artificial Sequence
  Description of Artificial Sequence Synthetic polypeptide
QVQLQESGGGLVQAGGSLRLSCAASARTFIYKMGWFRQAPGKERDFVASIMWSVGNNYYYTDSAKGRFTISRDIAKNTMYLQMDSLEPEDTGEYYCAAATTSTQWRYWGQGTQVTVSS

SEQ ID NO: 118 - protein, 115 aa, Artificial Sequence
  Description of Artificial Sequence Synthetic polypeptide
QVQLQESGGGWVQPGGSLRLSCAASGSIDNGYVMGWYRQAPGKQRELVATITSGTNTHYADSVKGRFTISRDNAKTTVYLQMNSLKPEDTAVYYCLARRLFTMYWGQGTQVTVSS

SEQ ID NO: 119 - protein, 116 aa, Artificial Sequence
  Description of Artificial Sequence Synthetic polypeptide
QVQLQESGGGLVQPGGSLRLSCSASGNRFSSSYVMGYYRQAPGKQRELVATVTTGGLTNYADSVKGRFTISRDNAKNTVYLQMNSLKPEDTAVYYCNARILLTNYWGQGTQVTVSS

SEQ ID NO: 120 - protein, 115 aa, Artificial Sequence
  Description of Artificial Sequence Synthetic polypeptide
QVQLQESGGGLAQTGDSLRLSCAASGRMFSGFVMGWYRQNPGKQRELVATITNGGLTHYGDSVKGRFTISRDNAKNTVYLQMNSLKSEDSAVYYCNARRLFTNYWGQGTQVTVSP

SEQ ID NO: 121 - protein, 122 aa, Artificial Sequence
  Description of Artificial Sequence Synthetic polypeptide
QVQLQESGGGLVQAGGSLRLSCAASGRTFEATYMGWFRQSPGKEREFVAAISWGGGTTYYGDSVKGRFTVSRDNAKNTAYLQMNSLKLEDTAVYSCAAATVDWGPRGPYRYWGQGTQVTVSS

SEQ ID NO: 122 - protein, 125 aa, Artificial Sequence
  Description of Artificial Sequence Synthetic polypeptide
QVQLQESGGGLVQAGGSLRLSCAASGRTFSTYYKGWFRQAPGKEREFLAAISDGGTYYADSVKGRFTISRDNAKNTVYLQMNSLKPEDTAVYYCAAQGVWRGTGSYTWQYSYDYWGQGTQVTVSS

SEQ ID NO: 123 - protein, 126 aa, Artificial Sequence
  Description of Artificial Sequence Synthetic polypeptide
QVQLQESGGGLVQAGGSLRLSCAASGRTFNRYAMGWFRQAPGKEREFVAAISWSGNTQYTDSVKGRFTISRDDAKNTVYLQMNSLKPEDTAVYYCALRIPASSSTTYYYADQYDYWGQGTQVTVSS

SEQ ID NO: 124 - protein, 115 aa, Artificial Sequence
  Description of Artificial Sequence Synthetic polypeptide
QVQLQESGGGLVQAGGSLRLSCAASGSISASYVMGWYRQTPGKQRELVATTTSGGTTRYADSVRGRFTISRDNARNTVYLQMNSLKPDDTAVYYCNARRLFLNYWGQGTQVTVSS

SEQ ID NO: 125 - protein, 115 aa, Artificial Sequence
  Description of Artificial Sequence Synthetic polypeptide
QVQLQQSGGGLVQPGGSLRLSCAASGRMFSGYVMGWYRQAPGKQRELVATITNGGLTNYADSVKGRFTISRDNAKNTVYLQMNSLKPDDTAVYYCNARRLWDIYWGQGTQVTVSS

SEQ ID NO: 126 - protein, 124 aa, Artificial Sequence
  Description of Artificial Sequence Synthetic polypeptide
QVQLQESGGGLVQAGGSLRLSCEASGRTFSSYRVGWFRQAPGKGREFVAAISATGGTTYYGDSVKGRFTISRDNAENTVSLQMNSLEPEDTAVYYCAATKGIVNYRVAGTYDAWGQGTQVTVSS

SEQ ID NO: 127 - protein, 115 aa, Artificial Sequence
  Description of Artificial Sequence Synthetic polypeptide
QVQLQESGGGLVQAGGSLRLSCSASGSISSSHVMGWYRQAPGKQRELVATITNGGLTHYADSVKGRFTISRDNAKNTVYLQMNSLKPDDTAVYYCNARRLWDNYWGQGTQVTVSS

SEQ ID NO: 128 - protein, 115 aa, Artificial Sequence
  Description of Artificial Sequence Synthetic polypeptide
QVQLQQSGGGLVQAGGSLRISCAASGSISSAYVMGWYRQAPGTQRELVATITSGGTTNYADSVKGRFTVSRDNAKNTVYLQMNSLKPDDTAVYYCNARRLWTTYWGQGTQVTVSS

SEQ ID NO: 129 - protein, 115 aa, Artificial Sequence
  Description of Artificial Sequence Synthetic polypeptide
QVQLQESGGGLVQPGESLRLSCAASTSGFSSYVMAWYRQAPGKQRELVASMTTGGLTNYADSVKGRFTISRDNAKNTVYLQMNSLKPEDTAAYYCNARRLWNAYWGQGTQVTVSS

SEQ ID NO: 130 - protein, 115 aa, Artificial Sequence
  Description of Artificial Sequence Synthetic polypeptide
QVQLQESGGGLVQAGGSLRLSCVSSGSISASHVMGWYRQAPGKQRELVATITSGGTTRYADSVKGRFTVSRDNAKNTVYLQMNDLKSEDTAVYYCHARRLWDTYWGQGTQVTVSS

SEQ ID NO: 131 - protein, 115 aa, Artificial Sequence
  Description of Artificial Sequence Synthetic polypeptide
QVQLQQSGGGLVQPGGSLRLSCAASGRIFSGHVMGWYRQAPGKQRELVATITNGGLTNYGDSVKGRFTISRDNAKNTVYLQMNSLKPDDTAVYYCNARRLWDTYWGQGTQVTVSS

SEQ ID NO: 132 - protein, 116 aa, Artificial Sequence
  Description of Artificial Sequence Synthetic polypeptide
QVQLQESGGGLVQAGGSLRLSCAASRRDFTTTTMAWYRQAPGKKRETVATVNTGGLTWYADFVKGRFTISRDDAVNTLLLQMDSLKPEDTAVYYCNAVLVRARGMWGQGTQVTVSS

SEQ ID NO: 133 - protein, 116 aa, Artificial Sequence
  Description of Artificial Sequence Synthetic polypeptide
QVQLQESGGGLVQPGGSLRLSCSASGNRFSSSYVMGWYRQGPGKQRELVATVTSGGMTHYGDSVKGRFTISRDNAKNTVYLHMNSLKPEDTGVYYCFARRLWDIHWGQGTQVTVSS

SEQ ID NO: 134 - protein, 115 aa, Artificial Sequence
  Description of Artificial Sequence Synthetic polypeptide
QVQLQESGGGLAQAGGSLGLSCAASETIDSSHVMGWYRQAPGKQRELVATITSGGLTNYADSAKGRFTISRDNAKNAVYLQMNSLKPEDTAVYYCHARRLFRVYWGQGTQVTVSS

SEQ ID NO: 135 - protein, 115 aa, Artificial Sequence
  Description of Artificial Sequence Synthetic polypeptide
QVQLQESGGGLVQAGGSLRLSCVASGSISNSHVMGWYRQAPGKERELVATLTSGGLTHFGDSVKGRFTISRDNAKNTIYLQMNSLKVEDTAVYYCNARRLLTSMWGQGTQVTVSP

SEQ ID NO: 136 - protein, 116 aa, Artificial Sequence
  Description of Artificial Sequence Synthetic polypeptide
QVQLQESGGGLVQPGGSLRLSCSASGNRFSSSYVLGWYRQAPGKQRELVATVTSGGLTHYGDSVKGRFTISRDNAKNTVYLQMNSLKPEDTAVYYCNARILLTNYWGQGTQVTVSS

SEQ ID NO: 137 - protein, 122 aa, Artificial Sequence
  Description of Artificial Sequence Synthetic polypeptide
QVQLQQSGGGLAQAGGSLRLSCAASGRTFNWYTMAWFRQAPGKEREFVAAIRLGSTVYGDSVKARFTISRDNAKSTVSLQMNSLKPEDTALYYCAVGITGDGTIQGGPYQYWGQGTQVTVSS

SEQ ID NO: 138 - protein, 115 aa, Artificial Sequence
  Description of Artificial Sequence Synthetic polypeptide
QVQLQESGGGLVQPGGSLRLSCAASGIISSAYVMGWYRQAPGKQRELVATITSGGTTRYADSVKGRITISRDNAKNTVLLQMNSLKPEDTAVYYCNARILLTNYWGQGTQVTVSS

SEQ ID NO: 139 - protein, 115 aa, Artificial Sequence
  Description of Artificial Sequence Synthetic polypeptide
QVQLQESGGGLVQAGGSLRLSCADSGRVFSTYVMGWYRQVPGKQRELVATITPGGLINYGDAVKGRFTISRDNAKNTVYLQMNSLKPADTAVYYCNARRLFAINWGGGTQVTVSS

SEQ ID NO: 140 - protein, 115 aa, Artificial Sequence
  Description of Artificial Sequence Synthetic polypeptide
QVQLQESGGGSVQPGGSLRLSCAASGSALSSNVLGWYRQAPGKQRELVATISSGGLTNYADSVKGRFTISRDNAKNTVYLQMNSLKPEDTAVYYCQSRRLFTVYWGQGTQVTVSS

SEQ ID NO: 141 - protein, 122 aa, Artificial Sequence
  Description of Artificial Sequence Synthetic polypeptide
QVQLQESGGGLVQAGESLRLSCADSGRTSSTYDMAWFRQAPGKEREFVAAISRDGGRLSYADSVKGRFTISRDNAKNTLSLQMNSLRPEDTAVYYCAAFSIRGGLRPSYKYWGQGTQVTVSS

SEQ ID NO: 142 - protein, 118 aa, Artificial Sequence
  Description of Artificial Sequence Synthetic polypeptide
QVQLQESGGGLVQPGGSLRLSCTASGSIFSLLNMGWYRQAPGKQRELVASITSRSYTNYADSVKGRFTISRDNTKNMVYLQMNSLKPEDTAVYYCNLNPADWGRLRNWGQGTQVTVSS

SEQ ID NO: 143 - protein, 116 aa, Artificial Sequence
  Description of Artificial Sequence Synthetic polypeptide
QVQLQESGGGVVQSGGSLRLSCAGPRSIFSGNAMAWYRQAPGKQRETVATVNTGGLTWYVDFVKGRFTISRDDAKNTLLLQMDSLKPEDTAVYYCNAVLVRARGMWGQGTQVTVSS

SEQ ID NO: 144 - protein, 124 aa, Artificial Sequence
  Description of Artificial Sequence Synthetic polypeptide
QVQLQESGGGLVQAGGSLRLSCAASGLTFGSYAMGWFRQAPGKEREFVAAIMRYSSRTYYTDSVKGRFTISRDNAKNTVNLQMNNLEPEDTAIYYCAAAKRLSIVTLPRQYEFWGQGTQVTVSS

SEQ ID NO: 145 - protein, 115 aa, Artificial Sequence
  Description of Artificial Sequence Synthetic polypeptide
QVQLQESGGGLVQPGGSLRLSCAASGSISGSYVMGWYRQAPGKQRELVATITSGGLTRYADSVKGRWTISRDNAKNTVYLQMNNLKLEDTAVYYCSARRIATTYWGQGTQVTVSS

SEQ ID NO: 146 - protein, 115 aa, Artificial Sequence
  Description of Artificial Sequence Synthetic polypeptide
QVQLQESGGGLVQPGGSLRLSCAASGSISSSYVMGWYRQAPGKQRDLVATITNAGNIHYGDSVKGRFTISRDNAKNTVSLQMNSLKPEDTAVYYCNARALWRAYWGQGTQVTVSS

SEQ ID NO: 147 - protein, 124 aa, Artificial Sequence
  Description of Artificial Sequence Synthetic polypeptide
QVQLQESGGGLVQAGGSLRLSCSASGRTFSVRAMGWFRQAPGKERESVAAIHQNTRTTLYADSVKGRFAISRDGTKNTVYLQMNSLKPEDTAVYYCAASDDYGLQIKEVAYKYWGQGTQVTVAS

SEQ ID NO: 148 - protein, 124 aa, Artificial Sequence
  Description of Artificial Sequence Synthetic polypeptide
QVQLQESGGGLVQAGGSLRLSCAASGLTFGSYAMGWFRQAPGKEREFVATIMRYSSRTYYTDSVKGRFTISRDNAKNTVNLQMNNLEPEDAAIYYCAAAKRLSIVALPRQYEFWGQGTQVTVSS

SEQ ID NO: 149 - protein, 124 aa, Artificial Sequence
  Description of Artificial Sequence Synthetic polypeptide
QVQLQQSGGGLVQAGGSLRLSCAASGLTFGSYAMGWFRQAPGKEREFVAAIMRYSSRTYYTDSVKGRFTISRDNAKNTVNLQMNNLEPEDTAIYYCAAAKRLSRVTLPREYEFWGQGTQVTVSS

SEQ ID NO: 150 - protein, 115 aa, Artificial Sequence; identical to SEQ ID NO: 129
  Description of Artificial Sequence Synthetic polypeptide

SEQ ID NO: 151 - protein, 9 aa, Artificial Sequence
  Description of Artificial Sequence Synthetic peptide
RFTFSTYPM

SEQ ID NO: 152 - protein, 9 aa, Artificial Sequence
  Description of Artificial Sequence Synthetic peptide
GRTFSRYAM

SEQ ID NO: 153 - protein, 9 aa, Artificial Sequence
  Description of Artificial Sequence Synthetic peptide
GSMFSINAM

SEQ ID NO: 154 - protein, 9 aa, Artificial Sequence
  Description of Artificial Sequence Synthetic peptide
GRTFSSYTM

SEQ ID NO: 155 - protein, 9 aa, Artificial Sequence
  Description of Artificial Sequence Synthetic peptide
GSFFSIYAM

SEQ ID NO: 156 - protein, 9 aa, Artificial Sequence
  Description of Artificial Sequence Synthetic peptide
GRTSSSFAM

SEQ ID NO: 157 - protein, 9 aa, Artificial Sequence
  Description of Artificial Sequence Synthetic peptide
GGTFSRLTM

SEQ ID NO: 158 - protein, 9 aa, Artificial Sequence
  Description of Artificial Sequence Synthetic peptide
GSMFNINAM

SEQ ID NO: 159 - protein, 9 aa, Artificial Sequence
  Description of Artificial Sequence Synthetic peptide
GSIFSGNVM

SEQ ID NO: 160 - protein, 9 aa, Artificial Sequence
  Description of Artificial Sequence Synthetic peptide
GSIFSRDAM

SEQ ID NO: 161 - protein, 9 aa, Artificial Sequence
  Description of Artificial Sequence Synthetic peptide
GGTFSRLTM

SEQ ID NO: 162 - protein, 9 aa, Artificial Sequence
  Description of Artificial Sequence Synthetic peptide
GTIFSINKM

SEQ ID NO: 163 - protein, 9 aa, Artificial Sequence
  Description of Artificial Sequence Synthetic peptide
GSIFSINAM

SEQ ID NO: 164 - protein, 9 aa, Artificial Sequence
  Description of Artificial Sequence Synthetic peptide
GSSFRSNAI

SEQ ID NO: 165 - protein, 9 aa, Artificial Sequence
  Description of Artificial Sequence Synthetic peptide
GSMFSRNAM

SEQ ID NO: 166 - protein, 9 aa, Artificial Sequence
  Description of Artificial Sequence Synthetic peptide
GLTFSSYAM

SEQ ID NO: 167 - protein, 9 aa, Artificial Sequence
  Description of Artificial Sequence Synthetic peptide
GRSGSTSFM

SEQ ID NO: 168 - protein, 9 aa, Artificial Sequence
  Description of Artificial Sequence Synthetic peptide
GSIFSFNAM

SEQ ID NO: 169 - protein, 9 aa, Artificial Sequence
  Description of Artificial Sequence Synthetic peptide
GGTFSRLTM

SEQ ID NO: 170 - protein, 9 aa, Artificial Sequence
  Description of Artificial Sequence Synthetic peptide
GGTFSRLTL

SEQ ID NO: 171 - protein, 9 aa, Artificial Sequence
  Description of Artificial Sequence Synthetic peptide
GRTFSTYSM

SEQ ID NO: 172 - protein, 9 aa, Artificial Sequence
  Description of Artificial Sequence Synthetic peptide
GRLFNINAM

SEQ ID NO: 173 - protein, 9 aa, Artificial Sequence
  Description of Artificial Sequence Synthetic peptide
GRTLSNYAM

SEQ ID NO: 174 - protein, 9 aa, Artificial Sequence
  Description of Artificial Sequence Synthetic peptide
GRTVSSLPM

SEQ ID NO: 175 - protein, 9 aa, Artificial Sequence
  Description of Artificial Sequence Synthetic peptide
GSMFNINAM

SEQ ID NO: 176 - protein, 9 aa, Artificial Sequence
  Description of Artificial Sequence Synthetic peptide
ETTLATNAM

SEQ ID NO: 177 - protein, 9 aa, Artificial Sequence
  Description of Artificial Sequence Synthetic peptide
GSSFRSNAI

SEQ ID NO: 178 - protein, 9 aa, Artificial Sequence
  Description of Artificial Sequence Synthetic peptide
GRTFSSYAM

SEQ ID NO: 179 - protein, 9 aa, Artificial Sequence
  Description of Artificial Sequence Synthetic peptide
GGTFSRLTI

SEQ ID NO: 180 - protein, 9 aa, Artificial Sequence
  Description of Artificial Sequence Synthetic peptide
GVTFSDYPM

SEQ ID NO: 181 - protein, 9 aa, Artificial Sequence
  Description of Artificial Sequence Synthetic peptide
GGTFSRLTI

SEQ ID NO: 182 - protein, 9 aa, Artificial Sequence
  Description of Artificial Sequence Synthetic peptide
GGTFSRLTM

SEQ ID NO: 183 - protein, 9 aa, Artificial Sequence
  Description of Artificial Sequence Synthetic peptide
GTIFRSKSM

SEQ ID NO: 184 - protein, 9 aa, Artificial Sequence
  Description of Artificial Sequence Synthetic peptide
GGSFSRLTL

SEQ ID NO: 185 - protein, 9 aa, Artificial Sequence
  Description of Artificial Sequence Synthetic peptide
GLTFSNYAM

SEQ ID NO: 186 - protein, 9 aa, Artificial Sequence
  Description of Artificial Sequence Synthetic peptide
GGTFSRLTM

SEQ ID NO: 187 - protein, 9 aa, Artificial Sequence
  Description of Artificial Sequence Synthetic peptide
GRTFSSYPM

SEQ ID NO: 188 - protein, 9 aa, Artificial Sequence
  Description of Artificial Sequence Synthetic peptide
GSIFSGNVM

SEQ ID NO: 189 - protein, 9 aa, Artificial Sequence
  Description of Artificial Sequence Synthetic peptide
GIVFSSIVM

SEQ ID NO: 190 - protein, 9 aa, Artificial Sequence
  Description of Artificial Sequence Synthetic peptide
GSIFSGNVM

SEQ ID NO: 191 - protein, 9 aa, Artificial Sequence
  Description of Artificial Sequence Synthetic peptide
GSISNSYVM

SEQ ID NO: 192 - protein, 9 aa, Artificial Sequence
  Description of Artificial Sequence Synthetic peptide
TVTFSRYAM

SEQ ID NO: 193 - protein, 9 aa, Artificial Sequence
  Description of Artificial Sequence Synthetic peptide
GSIFSGNVM

SEQ ID NO: 194 - protein, 9 aa, Artificial Sequence
  Description of Artificial Sequence Synthetic peptide
GSIFSGNVM

SEQ ID NO: 195 - protein, 9 aa, Artificial Sequence
  Description of Artificial Sequence Synthetic peptide
GSIFSGNVM

SEQ ID NO: 196 - protein, 9 aa, Artificial Sequence
  Description of Artificial Sequence Synthetic peptide
GSIFSGNVM

SEQ ID NO: 197 - protein, 9 aa, Artificial Sequence
  Description of Artificial Sequence Synthetic peptide
GSIFSGNVM

SEQ ID NO: 198 - protein, 9 aa, Artificial Sequence
  Description of Artificial Sequence Synthetic peptide
GIIFSSNVM

SEQ ID NO: 199 - protein, 9 aa, Artificial Sequence
  Description of Artificial Sequence Synthetic peptide
GSIASGNVL

SEQ ID NO: 200 - protein, 9 aa, Artificial Sequence
  Description of Artificial Sequence Synthetic peptide
GSIFSGNVL

SEQ ID NO: 201 - protein, 9 aa, Artificial Sequence
  Description of Artificial Sequence Synthetic peptide
GRTFSSYPM

SEQ ID NO: 202 - protein, 9 aa, Artificial Sequence
  Description of Artificial Sequence Synthetic peptide
GRTFSSYPM

SEQ ID NO: 203 - protein, 9 aa, Artificial Sequence
  Description of Artificial Sequence Synthetic peptide
GRTFSSYPM

SEQ ID NO: 204 - protein, 9 aa, Artificial Sequence
  Description of Artificial Sequence Synthetic peptide
GRTFSSYPM

SEQ ID NO: 205 - protein, 9 aa, Artificial Sequence
  Description of Artificial Sequence Synthetic peptide
GRTFSSYPM

SEQ ID NO: 206 - protein, 11 aa, Artificial Sequence
  Description of Artificial Sequence Synthetic peptide
DGAITGSYYVW

SEQ ID NO: 207 - protein, 9 aa, Artificial Sequence
  Description of Artificial Sequence Synthetic peptide
GFTFGSYAM

SEQ ID NO: 208 - protein, 8 aa, Artificial Sequence
  Description of Artificial Sequence Synthetic peptide
GSIFTSDV

SEQ ID NO: 209 - protein, 9 aa, Artificial Sequence
  Description of Artificial Sequence Synthetic peptide
GRTFSSYPM

SEQ ID NO: 210 - protein, 9 aa, Artificial Sequence
  Description of Artificial Sequence Synthetic peptide
GRTFSSYAM

SEQ ID NO: 211 - protein, 9 aa, Artificial Sequence
  Description of Artificial Sequence Synthetic peptide
GMLFSIRNM

SEQ ID NO: 212 - protein, 9 aa, Artificial Sequence
  Description of Artificial Sequence Synthetic peptide
MRTFNSRTI

SEQ ID NO: 213 - protein, 9 aa, Artificial Sequence
  Description of Artificial Sequence Synthetic peptide
GRTFSSYAM

SEQ ID NO: 214 - protein, 9 aa, Artificial Sequence
  Description of Artificial Sequence Synthetic peptide
EYIFSNFGM

SEQ ID NO: 215 - protein, 9 aa, Artificial Sequence
  Description of Artificial Sequence Synthetic peptide
GGTNSNYAM

SEQ ID NO: 216 - protein, 9 aa, Artificial Sequence
  Description of Artificial Sequence Synthetic peptide
GRTSSSSYL

SEQ ID NO: 217 - protein, 9 aa, Artificial Sequence
  Description of Artificial Sequence Synthetic peptide
GRTSGAWNM

SEQ ID NO: 218 - protein, 9 aa, Artificial Sequence
  Description of Artificial Sequence Synthetic peptide
GRTFSNYVI

SEQ ID NO: 219 - protein, 9 aa, Artificial Sequence
  Description of Artificial Sequence Synthetic peptide
GMLFSIRNM

SEQ ID NO: 220 - protein, 9 aa, Artificial Sequence
  Description of Artificial Sequence Synthetic peptide
GQTFTNSGM

SEQ ID NO: 221 - protein, 9 aa, Artificial Sequence
  Description of Artificial Sequence Synthetic peptide
GSIFSIDFM

SEQ ID NO: 222 - protein, 13 aa, Artificial Sequence
  Description of Artificial Sequence Synthetic peptide
GRTLSSYLSSYAM

SEQ ID NO: 223 - protein, 9 aa, Artificial Sequence
  Description of Artificial Sequence Synthetic peptide
GRTFSAYTM

SEQ ID NO: 224 - protein, 9 aa, Artificial Sequence
  Description of Artificial Sequence Synthetic peptide
GSISSSHVM

SEQ ID NO: 225 - protein, 9 aa, Artificial Sequence
  Description of Artificial Sequence Synthetic peptide
GSISSSFVM

SEQ ID NO: 226 - protein, 9 aa, Artificial Sequence
  Description of Artificial Sequence Synthetic peptide
RSIFSGNAM

SEQ ID NO: 227 - protein, 9 aa, Artificial Sequence
  Description of Artificial Sequence Synthetic peptide
GDRLSSYVM

SEQ ID NO: 228 - protein, 9 aa, Artificial Sequence
  Description of Artificial Sequence Synthetic peptide
GSVLSRYVM

SEQ ID NO: 229 - protein, 9 aa, Artificial Sequence
  Description of Artificial Sequence Synthetic peptide
GDAFSRYVM

SEQ ID NO: 230 - protein, 9 aa, Artificial Sequence
  Description of Artificial Sequence Synthetic peptide
GSISSSYVM

SEQ ID NO: 231 - protein, 9 aa, Artificial Sequence
  Description of Artificial Sequence Synthetic peptide
GSISSSHVM

SEQ ID NO: 232 - protein, 9 aa, Artificial Sequence
  Description of Artificial Sequence Synthetic peptide
ESMFRDHNM

SEQ ID NO: 233 - protein, 10 aa, Artificial Sequence
  Description of Artificial Sequence Synthetic peptide
GNRFSSSYVM

SEQ ID NO: 234 - protein, 9 aa, Artificial Sequence
  Description of Artificial Sequence Synthetic peptide
GIRLGSYVM

SEQ ID NO: 235 - protein, 9 aa, Artificial Sequence
  Description of Artificial Sequence Synthetic peptide
GIVFANHVM

SEQ ID NO: 236 - protein, 9 aa, Artificial Sequence
  Description of Artificial Sequence Synthetic peptide
GRTVGSYAM

SEQ ID NO: 237 - protein, 9 aa, Artificial Sequence
  Description of Artificial Sequence Synthetic peptide
GSIGSDYVL

SEQ ID NO: 238 - protein, 9 aa, Artificial Sequence
  Description of Artificial Sequence Synthetic peptide
GSIRSSNVM

SEQ ID NO: 239 - protein, 8 aa, Artificial Sequence
  Description of Artificial Sequence Synthetic peptide
ARTFIYKM

SEQ ID NO: 240 - protein, 9 aa, Artificial Sequence
  Description of Artificial Sequence Synthetic peptide
GSIDNGYVM

SEQ ID NO: 241 - protein, 10 aa, Artificial Sequence
  Description of Artificial Sequence Synthetic peptide
GNRFSSSYVM

SEQ ID NO: 242 - protein, 9 aa, Artificial Sequence
  Description of Artificial Sequence Synthetic peptide
GRMFSGFVM

SEQ ID NO: 243 - protein, 9 aa, Artificial Sequence
  Description of Artificial Sequence Synthetic peptide
GRTFEATYM

SEQ ID NO: 244 - protein, 9 aa, Artificial Sequence
  Description of Artificial Sequence Synthetic peptide
GRTFSTYYK

SEQ ID NO: 245 - protein, 9 aa, Artificial Sequence
  Description of Artificial Sequence Synthetic peptide
GRTFNRYAM

SEQ ID NO: 246 - protein, 9 aa, Artificial Sequence
  Description of Artificial Sequence Synthetic peptide
GSISASYVM

SEQ ID NO: 247 - protein, 9 aa, Artificial Sequence
  Description of Artificial Sequence Synthetic peptide
GRMFSGYVM

SEQ ID NO: 248 - protein, 9 aa, Artificial Sequence
  Description of Artificial Sequence Synthetic peptide
GRTFSSYRV

SEQ ID NO: 249 - protein, 9 aa, Artificial Sequence
  Description of Artificial Sequence Synthetic peptide
GSISSSHVM

SEQ ID NO: 250 - protein, 9 aa, Artificial Sequence
  Description of Artificial Sequence Synthetic peptide
GSISSAYVM

SEQ ID NO: 251 - protein, 9 aa, Artificial Sequence
  Description of Artificial Sequence Synthetic peptide
TSGFSSYVM

SEQ ID NO: 252 - protein, 9 aa, Artificial Sequence
  Description of Artificial Sequence Synthetic peptide
GSISASHVM

SEQ ID NO: 253 - protein, 9 aa, Artificial Sequence
  Description of Artificial Sequence Synthetic peptide
GRIFSGHVM

SEQ ID NO: 254 - protein, 9 aa, Artificial Sequence
  Description of Artificial Sequence Synthetic peptide
RRDFTTTTM

SEQ ID NO: 255 - protein, 10 aa, Artificial Sequence
  Description of Artificial Sequence Synthetic peptide
GNRFSSSYVM

SEQ ID NO: 256 - protein, 9 aa, Artificial Sequence
  Description of Artificial Sequence Synthetic peptide
ETIDSSHVM

SEQ ID NO: 257 - protein, 9 aa, Artificial Sequence
  Description of Artificial Sequence Synthetic peptide
GSISNSHVM

SEQ ID NO: 258 - protein, 10 aa, Artificial Sequence
  Description of Artificial Sequence Synthetic peptide
GNRFSSSYVL

SEQ ID NO: 259 - protein, 9 aa, Artificial Sequence
  Description of Artificial Sequence Synthetic peptide
GRTFNWYTM

SEQ ID NO: 260 - protein, 9 aa, Artificial Sequence
  Description of Artificial Sequence Synthetic peptide
GIISSAYVM

SEQ ID NO: 261 - protein, 9 aa, Artificial Sequence
  Description of Artificial Sequence Synthetic peptide
GRVFSTYVM

SEQ ID NO: 262 - protein, 9 aa, Artificial Sequence
  Description of Artificial Sequence Synthetic peptide
GSALSSNVL

SEQ ID NO: 263 - protein, 9 aa, Artificial Sequence
  Description of Artificial Sequence Synthetic peptide
GRTSSTYDM

SEQ ID NO: 264 - protein, 9 aa, Artificial Sequence
  Description of Artificial Sequence Synthetic peptide
GSIFSLLNM

SEQ ID NO: 265 - protein, 9 aa, Artificial Sequence
  Description of Artificial Sequence Synthetic peptide
RSIFSGNAM

SEQ ID NO: 266 - protein, 9 aa, Artificial Sequence
  Description of Artificial Sequence Synthetic peptide
GLTFGSYAM

SEQ ID NO: 267 - protein, 9 aa, Artificial Sequence
  Description of Artificial Sequence Synthetic peptide
GSISGSYVM

SEQ ID NO: 268 - protein, 9 aa, Artificial Sequence
  Description of Artificial Sequence Synthetic peptide
GSISSSYVM

SEQ ID NO: 269 - protein, 9 aa, Artificial Sequence
  Description of Artificial Sequence Synthetic peptide
GRTFSVRAM

SEQ ID NO: 270 - protein, 9 aa, Artificial Sequence
  Description of Artificial Sequence Synthetic peptide
GLTFGSYAM

SEQ ID NO: 271 - protein, 9 aa, Artificial Sequence
  Description of Artificial Sequence Synthetic peptide
GLTFGSYAM

SEQ ID NO: 272 - protein, 9 aa, Artificial Sequence
  Description of Artificial Sequence Synthetic peptide
TSGFSSYVM

SEQ ID NO: 273 - protein, 8 aa, Artificial Sequence
  Description of Artificial Sequence Synthetic peptide
ISVGGGIK

SEQ ID NO: 274 - protein, 8 aa, Artificial Sequence
  Description of Artificial Sequence Synthetic peptide
VDWSGGST

SEQ ID NO: 275 - protein, 7 aa, Artificial Sequence
  Description of Artificial Sequence Synthetic peptide
ISRGGIT

SEQ ID NO: 276 - protein, 8 aa, Artificial Sequence
  Description of Artificial Sequence Synthetic peptide
IRWSGGSR

SEQ ID NO: 277 - protein, 7 aa, Artificial Sequence
  Description of Artificial Sequence Synthetic peptide
ITSGSET

SEQ ID NO: 278 - protein, 8 aa, Artificial Sequence
  Description of Artificial Sequence Synthetic peptide
ITRTGGRT

SEQ ID NO: 279 - protein, 8 aa, Artificial Sequence
  Description of Artificial Sequence Synthetic peptide
VSWVAETT

SEQ ID NO: 280 - protein, 7 aa, Artificial Sequence
  Description of Artificial Sequence Synthetic peptide
ISSGGIT

SEQ ID NO: 281 - protein, 7 aa, Artificial Sequence
  Description of Artificial Sequence Synthetic peptide
ISSGGIT

SEQ ID NO: 282 - protein, 7 aa, Artificial Sequence
  Description of Artificial Sequence Synthetic peptide
ISSGGIT

SEQ ID NO: 283 - protein, 8 aa, Artificial Sequence
  Description of Artificial Sequence Synthetic peptide
VSWVAETT

SEQ ID NO: 284 - protein, 7 aa, Artificial Sequence
  Description of Artificial Sequence Synthetic peptide
ARSGGII

SEQ ID NO: 285 - protein, 7 aa, Artificial Sequence
  Description of Artificial Sequence Synthetic peptide
ISSGGIT

SEQ ID NO: 286 - protein, 7 aa, Artificial Sequence
  Description of Artificial Sequence Synthetic peptide
ITRSGST

SEQ ID NO: 287 - protein, 7 aa, Artificial Sequence
  Description of Artificial Sequence Synthetic peptide
ISSGGIT

SEQ ID NO: 288 - protein, 8 aa, Artificial Sequence
  Description of Artificial Sequence Synthetic peptide
ISWSGKST

SEQ ID NO: 289 - protein, 8 aa, Artificial Sequence
  Description of Artificial Sequence Synthetic peptide
IRWSSGMT

SEQ ID NO: 290 - protein, 7 aa, Artificial Sequence
  Description of Artificial Sequence Synthetic peptide
ITKGGST

SEQ ID NO: 291 - protein, 8 aa, Artificial Sequence
  Description of Artificial Sequence Synthetic peptide
VSWVAETT

SEQ ID NO: 292 - protein, 8 aa, Artificial Sequence
  Description of Artificial Sequence Synthetic peptide
VSWVAEMT

SEQ ID NO: 293 - protein, 8 aa, Artificial Sequence
  Description of Artificial Sequence Synthetic peptide
IRWSGGSR

SEQ ID NO: 294 - protein, 7 aa, Artificial Sequence
  Description of Artificial Sequence Synthetic peptide
ISSGGIT

SEQ ID NO: 295 - protein, 8 aa, Artificial Sequence
  Description of Artificial Sequence Synthetic peptide
ISRSGMST

SEQ ID NO: 296 - protein, 8 aa, Artificial Sequence
  Description of Artificial Sequence Synthetic peptide
LNWSGTST

SEQ ID NO: 297 - protein, 7 aa, Artificial Sequence
  Description of Artificial Sequence Synthetic peptide
ISSGGIT

SEQ ID NO: 298 - protein, 10 aa, Artificial Sequence
  Description of Artificial Sequence Synthetic peptide
ISSVSSGGIT

SEQ ID NO: 299 - protein, 7 aa, Artificial Sequence
  Description of Artificial Sequence Synthetic peptide
ITRSGST

SEQ ID NO: 300 - protein, 8 aa, Artificial Sequence
  Description of Artificial Sequence Synthetic peptide
IKWSGGSR

SEQ ID NO: 301 - protein, 8 aa, Artificial Sequence
  Description of Artificial Sequence Synthetic peptide
VSWVAETT

SEQ ID NO: 302 - protein, 8 aa, Artificial Sequence
  Description of Artificial Sequence Synthetic peptide
ISAGGGLI

SEQ ID NO: 303 - protein, 8 aa, Artificial Sequence
  Description of Artificial Sequence Synthetic peptide
VSWVAQTT

SEQ ID NO: 304 - protein, 8 aa, Artificial Sequence
  Description of Artificial Sequence Synthetic peptide
VSWVAGTT

SEQ ID NO: 305 - protein, 7 aa, Artificial Sequence
  Description of Artificial Sequence Synthetic peptide
ISGLGHT

SEQ ID NO: 306 - protein, 8 aa, Artificial Sequence
  Description of Artificial Sequence Synthetic peptide
VSWVAETT

SEQ ID NO: 307 - protein, 8 aa, Artificial Sequence
  Description of Artificial Sequence Synthetic peptide
ISWSGKST

SEQ ID NO: 308 - protein, 8 aa, Artificial Sequence
  Description of Artificial Sequence Synthetic peptide
VSWVAETT

SEQ ID NO: 309 - protein, 8 aa, Artificial Sequence
  Description of Artificial Sequence Synthetic peptide
ISAGGGLI

SEQ ID NO: 310 - protein, 7 aa, Artificial Sequence
  Description of Artificial Sequence Synthetic peptide
ITGGGIT

SEQ ID NO: 311 - protein, 7 aa, Artificial Sequence
  Description of Artificial Sequence Synthetic peptide
ITNGGLV

SEQ ID NO: 312 - protein, 7 aa, Artificial Sequence
  Description of Artificial Sequence Synthetic peptide
ITGGGIT

SEQ ID NO: 313 - protein, 7 aa, Artificial Sequence
  Description of Artificial Sequence Synthetic peptide
ITSGGLT

SEQ ID NO: 314 - protein, 8 aa, Artificial Sequence
  Description of Artificial Sequence Synthetic peptide
ISGSGHRT

SEQ ID NO: 315 - protein, 7 aa, Artificial Sequence
  Description of Artificial Sequence Synthetic peptide
MTGGGVT

SEQ ID NO: 316 - protein, 7 aa, Artificial Sequence
  Description of Artificial Sequence Synthetic peptide
ITGGGIT

SEQ ID NO: 317 - protein, 7 aa, Artificial Sequence
  Description of Artificial Sequence Synthetic peptide
ITGGGIT

SEQ ID NO: 318 - protein, 7 aa, Artificial Sequence
  Description of Artificial Sequence Synthetic peptide
ITGGGVT

SEQ ID NO: 319 - protein, 7 aa, Artificial Sequence
  Description of Artificial Sequence Synthetic peptide
MTGGGVT

SEQ ID NO: 320 - protein, 7 aa, Artificial Sequence
  Description of Artificial Sequence Synthetic peptide
RTSGGLT

SEQ ID NO: 321 - protein, 7 aa, Artificial Sequence
  Description of Artificial Sequence Synthetic peptide
ITSGGLT

SEQ ID NO: 322 - protein, 7 aa, Artificial Sequence
  Description of Artificial Sequence Synthetic peptide
ITGGGIT

SEQ ID NO: 323 - protein, 8 aa, Artificial Sequence
  Description of Artificial Sequence Synthetic peptide
ISRSGDPG

SEQ ID NO: 324 - protein, 8 aa, Artificial Sequence
  Description of Artificial Sequence Synthetic peptide
ISRSGVPG

SEQ ID NO: 325 - protein, 8 aa, Artificial Sequence
  Description of Artificial Sequence Synthetic peptide
ISRSGVSG

SEQ ID NO: 326 - protein, 8 aa, Artificial Sequence
  Description of Artificial Sequence Synthetic peptide
ISRSGNPG

SEQ ID NO: 327 - protein, 8 aa, Artificial Sequence
  Description of Artificial Sequence Synthetic peptide
INRNGNIP

SEQ ID NO: 328 - protein, 7 aa, Artificial Sequence
  Description of Artificial Sequence Synthetic peptide
ITYDGST

SEQ ID NO: 329 - protein, 8 aa, Artificial Sequence
  Description of Artificial Sequence Synthetic peptide
INSGDGRI

SEQ ID NO: 330 - protein, 7 aa, Artificial Sequence
  Description of Artificial Sequence Synthetic peptide
MTSAGTT

SEQ ID NO: 331 - protein, 8 aa, Artificial Sequence
  Description of Artificial Sequence Synthetic peptide
ISRSGDPG

SEQ ID NO: 332 - protein, 8 aa, Artificial Sequence
  Description of Artificial Sequence Synthetic peptide
ISRSGNPG

SEQ ID NO: 333 - protein, 7 aa, Artificial Sequence
  Description of Artificial Sequence Synthetic peptide
IGSSGNT

SEQ ID NO: 334 - protein, 7 aa, Artificial Sequence
  Description of Artificial Sequence Synthetic peptide
IAWTGGN

SEQ ID NO: 335 - protein, 8 aa, Artificial Sequence
  Description of Artificial Sequence Synthetic peptide
VSWSGGST

SEQ ID NO: 336 - protein, 8 aa, Artificial Sequence
  Description of Artificial Sequence Synthetic peptide
ISRSGSRM

SEQ ID NO: 337 - protein, 8 aa, Artificial Sequence
  Description of Artificial Sequence Synthetic peptide
LSWSGYNT

SEQ ID NO: 338 - protein, 8 aa, Artificial Sequence
  Description of Artificial Sequence Synthetic peptide
IRWSDGST

SEQ ID NO: 339 - protein, 8 aa, Artificial Sequence
  Description of Artificial Sequence Synthetic peptide
ISGSGRTT

SEQ ID NO: 340 - protein, 12 aa, Artificial Sequence
  Description of Artificial Sequence Synthetic peptide
VGRGINSAYHAT

SEQ ID NO: 341 - protein, 7 aa, Artificial Sequence
  Description of Artificial Sequence Synthetic peptide
IGTSGAT

SEQ ID NO: 342 - protein, 8 aa, Artificial Sequence
  Description of Artificial Sequence Synthetic peptide
VSRSGLGR

SEQ ID NO: 343 - protein, 6 aa, Artificial Sequence
  Description of Artificial Sequence Synthetic peptide
IRGGNT

SEQ ID NO: 344 - protein, 8 aa, Artificial Sequence
  Description of Artificial Sequence Synthetic peptide
ITWNGDRT

SEQ ID NO: 345 - protein, 7 aa, Artificial Sequence
  Description of Artificial Sequence Synthetic peptide
MSRGSAA

SEQ ID NO: 346 - protein, 7 aa, Artificial Sequence
  Description of Artificial Sequence Synthetic peptide
ITSGGST

SEQ ID NO: 347 - protein, 7 aa, Artificial Sequence
  Description of Artificial Sequence Synthetic peptide
ITSGGST

SEQ ID NO: 348 - protein, 7 aa, Artificial Sequence
  Description of Artificial Sequence Synthetic peptide
VNTGGLT

SEQ ID NO: 349 - protein, 7 aa, Artificial Sequence
  Description of Artificial Sequence Synthetic peptide
VTSGGRT

SEQ ID NO: 350 - protein, 7 aa, Artificial Sequence
  Description of Artificial Sequence Synthetic peptide
ITSGGIT

SEQ ID NO: 351 - protein, 7 aa, Artificial Sequence
  Description of Artificial Sequence Synthetic peptide
ITSGGST

SEQ ID NO: 352 - protein, 7 aa, Artificial Sequence
  Description of Artificial Sequence Synthetic peptide
ITSGGST

SEQ ID NO: 353 - protein, 7 aa, Artificial Sequence
  Description of Artificial Sequence Synthetic peptide
ITSGGST

SEQ ID NO: 354 - protein, 7 aa, Artificial Sequence
  Description of Artificial Sequence Synthetic peptide
ISRGGLI

SEQ ID NO: 355 - protein, 7 aa, Artificial Sequence
  Description of Artificial Sequence Synthetic peptide
VTSGGLT

SEQ ID NO: 356 - protein, 7 aa, Artificial Sequence
  Description of Artificial Sequence Synthetic peptide
VTSGGTT

SEQ ID NO: 357 - protein, 7 aa, Artificial Sequence
  Description of Artificial Sequence Synthetic peptide
ITNGGLI

SEQ ID NO: 358 - protein, 8 aa, Artificial Sequence
  Description of Artificial Sequence Synthetic peptide
IGWSGAST

SEQ ID NO: 359 - protein, 7 aa, Artificial Sequence
  Description of Artificial Sequence Synthetic peptide
ITSGGLT

SEQ ID NO: 360 - protein, 7 aa, Artificial Sequence
  Description of Artificial Sequence Synthetic peptide
MTAGGLT

SEQ ID NO: 361 - protein, 9 aa, Artificial Sequence
  Description of Artificial Sequence Synthetic peptide
IMWSVGNNY

SEQ ID NO: 362 - protein, 7 aa, Artificial Sequence
  Description of Artificial Sequence Synthetic peptide
ITSGTNT

SEQ ID NO: 363 - protein, 7 aa, Artificial Sequence
  Description of Artificial Sequence Synthetic peptide
VTTGGLT

SEQ ID NO: 364 - protein, 7 aa, Artificial Sequence
  Description of Artificial Sequence Synthetic peptide
ITNGGLT

SEQ ID NO: 365 - protein, 8 aa, Artificial Sequence
  Description of Artificial Sequence Synthetic peptide
ISWGGGTT

SEQ ID NO: 366 - protein, 6 aa, Artificial Sequence
  Description of Artificial Sequence Synthetic peptide
ISDGGT

SEQ ID NO: 367 - protein, 7 aa, Artificial Sequence
  Description of Artificial Sequence Synthetic peptide
ISWSGNT

SEQ ID NO: 368 - protein, 7 aa, Artificial Sequence
  Description of Artificial Sequence Synthetic peptide
TTSGGTT

SEQ ID NO: 369 - protein, 7 aa, Artificial Sequence
  Description of Artificial Sequence Synthetic peptide
ITNGGLT

SEQ ID NO: 370 - protein, 8 aa, Artificial Sequence
  Description of Artificial Sequence Synthetic peptide
ISATGGTT

SEQ ID NO: 371 - protein, 8 aa, Artificial Sequence
  Description of Artificial Sequence Synthetic peptide
ITNGGLTH

SEQ ID NO: 372 - protein, 7 aa, Artificial Sequence
  Description of Artificial Sequence Synthetic peptide
ITSGGTT

SEQ ID NO: 373 - protein, 7 aa, Artificial Sequence
  Description of Artificial Sequence Synthetic peptide
MTTGGLT

SEQ ID NO: 374 - protein, 7 aa, Artificial Sequence
  Description of Artificial Sequence Synthetic peptide
ITSGGTT

SEQ ID NO: 375 - protein, 7 aa, Artificial Sequence
  Description of Artificial Sequence Synthetic peptide
ITNGGLT

SEQ ID NO: 376 - protein, 7 aa, Artificial Sequence
  Description of Artificial Sequence Synthetic peptide
VNTGGLT

SEQ ID NO: 377 - protein, 7 aa, Artificial Sequence
  Description of Artificial Sequence Synthetic peptide
VTSGGMT

SEQ ID NO: 378 - protein, 7 aa, Artificial Sequence
  Description of Artificial Sequence Synthetic peptide
ITSGGLT

SEQ ID NO: 379 - protein, 7 aa, Artificial Sequence
  Description of Artificial Sequence Synthetic peptide
LTSGGLT

SEQ ID NO: 380 - protein, 7 aa, Artificial Sequence
  Description of Artificial Sequence Synthetic peptide
VTSGGLT

SEQ ID NO: 381 - protein, 6 aa, Artificial Sequence
  Description of Artificial Sequence Synthetic peptide
IRLGST

SEQ ID NO: 382 - protein, 7 aa, Artificial Sequence
  Description of Artificial Sequence Synthetic peptide
ITSGGTT

SEQ ID NO: 383 - protein, 7 aa, Artificial Sequence
  Description of Artificial Sequence Synthetic peptide
ITPGGLI

SEQ ID NO: 384 - protein, 7 aa, Artificial Sequence
  Description of Artificial Sequence Synthetic peptide
ISSGGLT

SEQ ID NO: 385 - protein, 8 aa, Artificial Sequence
  Description of Artificial Sequence Synthetic peptide
ISRDGGRL

SEQ ID NO: 386 - protein, 7 aa, Artificial Sequence
  Description of Artificial Sequence Synthetic peptide
ITSRSYT

SEQ ID NO: 387 - protein, 7 aa, Artificial Sequence
  Description of Artificial Sequence Synthetic peptide
VNTGGLT

SEQ ID NO: 388 - protein, 8 aa, Artificial Sequence
  Description of Artificial Sequence Synthetic peptide
IMRYSSRT

SEQ ID NO: 389 - protein, 7 aa, Artificial Sequence
  Description of Artificial Sequence Synthetic peptide
ITSGGLT

SEQ ID NO: 390 - protein, 7 aa, Artificial Sequence
  Description of Artificial Sequence Synthetic peptide
ITNAGNI

SEQ ID NO: 391 - protein, 8 aa, Artificial Sequence
  Description of Artificial Sequence Synthetic peptide
IHQNTRTT

SEQ ID NO: 392 - protein, 8 aa, Artificial Sequence
  Description of Artificial Sequence Synthetic peptide
IMRYSSRT

SEQ ID NO: 393 - protein, 8 aa, Artificial Sequence
  Description of Artificial Sequence Synthetic peptide
IMRYSSRT

SEQ ID NO: 394 - protein, 7 aa, Artificial Sequence
  Description of Artificial Sequence Synthetic peptide
MTTGGLT

SEQ ID NO: 395 - protein, 11 aa, Artificial Sequence
  Description of Artificial Sequence Synthetic peptide
AKGGKTSYTRE

SEQ ID NO: 396 - protein, 22 aa, Artificial Sequence
  Description of Artificial Sequence Synthetic peptide
AARARDVYGRAWYVEDSSTYDY

SEQ ID NO: 397 - protein, 11 aa, Artificial Sequence
  Description of Artificial Sequence Synthetic peptide
NAENRRLGDDF

SEQ ID NO: 398 - protein, 16 aa, Artificial Sequence
  Description of Artificial Sequence Synthetic peptide
AADRDGYSSYAHQYDY

SEQ ID NO: 399 - protein, 11 aa, Artificial Sequence
  Description of Artificial Sequence Synthetic peptide
NRWAPLTRLDY

SEQ ID NO: 400 - protein, 23 aa, Artificial Sequence
  Description of Artificial Sequence Synthetic peptide
AARWATATSNSIRVYYNEGQYDY

SEQ ID NO: 401 - protein, 18 aa, Artificial Sequence
  Description of Artificial Sequence Synthetic peptide
AAGPRDMIRSRNIRSYDS

SEQ ID NO: 402 - protein, 11 aa, Artificial Sequence
  Description of Artificial Sequence Synthetic peptide
NAENRRLGDDY

SEQ ID NO: 403 - protein, 11 aa, Artificial Sequence
  Description of Artificial Sequence Synthetic peptide
NLENRRLGDDY

SEQ ID NO: 404 - protein, 11 aa, Artificial Sequence
  Description of Artificial Sequence Synthetic peptide
NAENRRLGDDY

SEQ ID NO: 405 - protein, 18 aa, Artificial Sequence
  Description of Artificial Sequence Synthetic peptide
AAGPRDMIRSRNIKSYDS

SEQ ID NO: 406 - protein, 14 aa, Artificial Sequence
  Description of Artificial Sequence Synthetic peptide
NALIHTRYDRVTGY

SEQ ID NO: 407 - protein, 12 aa, Artificial Sequence
  Description of Artificial Sequence Synthetic peptide
NAENPRLGDDYW

SEQ ID NO: 408 - protein, 14 aa, Artificial Sequence
  Description of Artificial Sequence Synthetic peptide
HDETMKLISVKNDY

SEQ ID NO: 409 - protein, 11 aa, Artificial Sequence
  Description of Artificial Sequence Synthetic peptide
NAENRRLGDDY

SEQ ID NO: 410 - protein, 19 aa, Artificial Sequence
  Description of Artificial Sequence Synthetic peptide
AADYQRLGLLRVGVAEYDY

SEQ ID NO: 411 - protein, 18 aa, Artificial Sequence
  Description of Artificial Sequence Synthetic peptide
AADNYPLHIGHQDHEVDY

SEQ ID NO: 412 - protein, 15 aa, Artificial Sequence
  Description of Artificial Sequence Synthetic peptide
NVKTLRSALFPGYEY

SEQ ID NO: 413 - protein, 18 aa, Artificial Sequence
  Description of Artificial Sequence Synthetic peptide
AAGPRDMIRSRNIRSYDS

SEQ ID NO: 414 - protein, 18 aa, Artificial Sequence
  Description of Artificial Sequence Synthetic peptide
AAGPRDMVRSRNIRSYDS

SEQ ID NO: 415 - protein, 16 aa, Artificial Sequence
  Description of Artificial Sequence Synthetic peptide
AADRDGYSSYAHQYDY

SEQ ID NO: 416 - protein, 11 aa, Artificial Sequence
  Description of Artificial Sequence Synthetic peptide
NAENRGLGDDY

SEQ ID NO: 417 - protein, 21 aa, Artificial Sequence
  Description of Artificial Sequence Synthetic peptide
AARGGLPNPSRTYGFEEQYDY

SEQ ID NO: 418 - protein, 20 aa, Artificial Sequence
  Description of Artificial Sequence Synthetic peptide
AAKGAIYYSYSPRNNNNYDV

SEQ ID NO: 419 - protein, 10 aa, Artificial Sequence
  Description of Artificial Sequence Synthetic peptide
NAENRLGDDY

SEQ ID NO: 420 - protein, 9 aa, Artificial Sequence
  Description of Artificial Sequence Synthetic peptide
NGVRRGRSY

SEQ ID NO: 421 - protein, 14 aa, Artificial Sequence
  Description of Artificial Sequence Synthetic peptide
HDETMKLITGKNDY

SEQ ID NO: 422 - protein, 16 aa, Artificial Sequence
  Description of Artificial Sequence Synthetic peptide
AADRDGYSRYAHQYDY

SEQ ID NO: 423 - protein, 18 aa, Artificial Sequence
  Description of Artificial Sequence Synthetic peptide
AAGPRDMIRSRNIRSYDS

SEQ ID NO: 424 - protein, 10 aa, Artificial Sequence
  Description of Artificial Sequence Synthetic peptide
NLKTSYFWPW

SEQ ID NO: 425 - protein, 18 aa, Artificial Sequence
  Description of Artificial Sequence Synthetic peptide
AAGPQDMIRSRNIRSYIS

SEQ ID NO: 426 - protein, 18 aa, Artificial Sequence
  Description of Artificial Sequence Synthetic peptide
AAGPRDMIRSRNIRSYDS

SEQ ID NO: 427 - protein, 8 aa, Artificial Sequence
  Description of Artificial Sequence Synthetic peptide
NTFTAAFS

SEQ ID NO: 428 - protein, 18 aa, Artificial Sequence
  Description of Artificial Sequence Synthetic peptide
AAGPRDMIRSRNIRSYAS

SEQ ID NO: 429 - protein, 19 aa, Artificial Sequence
  Description of Artificial Sequence Synthetic peptide
AAEYQRLGLLRDGVADYSY

SEQ ID NO: 430 - protein, 18 aa, Artificial Sequence
  Description of Artificial Sequence Synthetic peptide
AAGPRDMIRSRNIRSYVS

SEQ ID NO: 431 - protein, 9 aa, Artificial Sequence
  Description of Artificial Sequence Synthetic peptide
NLKTSYFWP

SEQ ID NO: 432 - protein, 9 aa, Artificial Sequence
  Description of Artificial Sequence Synthetic peptide
NYRRIMQQY

SEQ ID NO: 433 - protein, 9 aa, Artificial Sequence
  Description of Artificial Sequence Synthetic peptide
NARRIMTSY

SEQ ID NO: 434 - protein, 9 aa, Artificial Sequence
  Description of Artificial Sequence Synthetic peptide
HYRRIMQQY

SEQ ID NO: 435 - protein, 9 aa, Artificial Sequence
  Description of Artificial Sequence Synthetic peptide
NARVIFTTY

SEQ ID NO: 436 - protein, 18 aa, Artificial Sequence
  Description of Artificial Sequence Synthetic peptide
AGDLVAKFDSAYRVSYDS

SEQ ID NO: 437 - protein, 9 aa, Artificial Sequence
  Description of Artificial Sequence Synthetic peptide
HYRRIMQQY

SEQ ID NO: 438 - protein, 9 aa, Artificial Sequence
  Description of Artificial Sequence Synthetic peptide
FYRRIMQQY

SEQ ID NO: 439 - protein, 9 aa, Artificial Sequence
  Description of Artificial Sequence Synthetic peptide
LYRRIMQQY

SEQ ID NO: 440 - protein, 9 aa, Artificial Sequence
  Description of Artificial Sequence Synthetic peptide
HYRRIMQQS

SEQ ID NO: 441 - protein, 9 aa, Artificial Sequence
  Description of Artificial Sequence Synthetic peptide
HYRRIMQQY

SEQ ID NO: 442 - protein, 9 aa, Artificial Sequence
  Description of Artificial Sequence Synthetic peptide
NARRLFTNY

SEQ ID NO: 443 - protein, 9 aa, Artificial Sequence
  Description of Artificial Sequence Synthetic peptide
NYRRLATGY

SEQ ID NO: 444 - protein, 9 aa, Artificial Sequence
  Description of Artificial Sequence Synthetic peptide
HYRRIMQQY

SEQ ID NO: 445 - protein, 12 aa, Artificial Sequence
  Description of Artificial Sequence Synthetic peptide
AARQIYSNNYSY

SEQ ID NO: 446 - protein, 12 aa, Artificial Sequence
  Description of Artificial Sequence Synthetic peptide
AARSIYSNNYSY

SEQ ID NO: 447 - protein, 12 aa, Artificial Sequence
  Description of Artificial Sequence Synthetic peptide
AARSIYSNNYSY

SEQ ID NO: 448 - protein, 12 aa, Artificial Sequence
  Description of Artificial Sequence Synthetic peptide
AARQIYSNNYSY

SEQ ID NO: 449 - protein, 12 aa, Artificial Sequence
  Description of Artificial Sequence Synthetic peptide
AARTIYDSHYTS

SEQ ID NO: 450 - protein, 22 aa, Artificial Sequence
  Description of Artificial Sequence Synthetic peptide
ARAGEEYICSGYGCHGSLGLDY

SEQ ID NO: 451 - protein, 9 aa, Artificial Sequence
  Description of Artificial Sequence Synthetic peptide
ATGIRTPII

SEQ ID NO: 452 - protein, 5 aa, Artificial Sequence
  Description of Artificial Sequence Synthetic peptide
GVGRF

SEQ ID NO: 453 - protein, 12 aa, Artificial Sequence
  Description of Artificial Sequence Synthetic peptide
AAREIYSNNYSY

SEQ ID NO: 454 - protein, 12 aa, Artificial Sequence
  Description of Artificial Sequence Synthetic peptide
AARQIYSNNYSY

SEQ ID NO: 455 - protein, 5 aa, Artificial Sequence
  Description of Artificial Sequence Synthetic peptide
NALNY

SEQ ID NO: 456 - protein, 19 aa, Artificial Sequence
  Description of Artificial Sequence Synthetic peptide
AAQTRPYDLPSIRPDDYAS

SEQ ID NO: 457 - protein, 19 aa, Artificial Sequence
  Description of Artificial Sequence Synthetic peptide
AAQRVMDYYRPRTESAYAY

SEQ ID NO: 458 - protein, 20 aa, Artificial Sequence
  Description of Artificial Sequence Synthetic peptide
AAVYGQYSYHYSSDSKQYSY

SEQ ID NO: 459 - protein, 22 aa, Artificial Sequence
  Description of Artificial Sequence Synthetic peptide
AARLSGRTAGSRTYYAEGQYDY

SEQ ID NO: 460 - protein, 15 aa, Artificial Sequence
  Description of Artificial Sequence Synthetic peptide
AAATTDWGPRGPYNY

SEQ ID NO: 461 - protein, 15 aa, Artificial Sequence
  Description of Artificial Sequence Synthetic peptide
AASTFDWGPRGPYRL

SEQ ID NO: 462 - protein, 17 aa, Artificial Sequence
  Description of Artificial Sequence Synthetic peptide
AVTSRWGQFDRTDFNSW

SEQ ID NO: 463 - protein, 5 aa, Artificial Sequence
  Description of Artificial Sequence Synthetic peptide
NALNY

SEQ ID NO: 464 - protein, 15 aa, Artificial Sequence
  Description of Artificial Sequence Synthetic peptide
AATTLDWGPRGPYRY

SEQ ID NO: 465 - protein, 16 aa, Artificial Sequence
  Description of Artificial Sequence Synthetic peptide
NAQITMRGGTWSTSEY

SEQ ID NO: 466 - protein, 20 aa, Artificial Sequence
  Description of Artificial Sequence Synthetic peptide
AADTVGRWRSTLSVRDEYDY

SEQ ID NO: 467 - protein, 18 aa, Artificial Sequence
  Description of Artificial Sequence Synthetic peptide
AGGSPGSSQIATPEAYTY

SEQ ID NO: 468 - protein, 9 aa, Artificial Sequence
  Description of Artificial Sequence Synthetic peptide
HARRLWNTY

SEQ ID NO: 469 - protein, 9 aa, Artificial Sequence
  Description of Artificial Sequence Synthetic peptide
HARRLWNTY

SEQ ID NO: 470 - protein, 10 aa, Artificial Sequence
  Description of Artificial Sequence Synthetic peptide
NAVLVRARGM

SEQ ID NO: 471 - protein, 9 aa, Artificial Sequence
  Description of Artificial Sequence Synthetic peptide
NARILFTNY

SEQ ID NO: 472 - protein, 9 aa, Artificial Sequence
  Description of Artificial Sequence Synthetic peptide
NARALWNTY

SEQ ID NO: 473 - protein, 9 aa, Artificial Sequence
  Description of Artificial Sequence Synthetic peptide
HARRLWNTY

SEQ ID NO: 474 - protein, 9 aa, Artificial Sequence
  Description of Artificial Sequence Synthetic peptide
HARRLWNTY

SEQ ID NO: 475 - protein, 9 aa, Artificial Sequence
  Description of Artificial Sequence Synthetic peptide
HARRLWDTY

SEQ ID NO: 476 - protein, 9 aa, Artificial Sequence
  Description of Artificial Sequence Synthetic peptide
NARRLLTTV

SEQ ID NO: 477 - protein, 9 aa, Artificial Sequence
  Description of Artificial Sequence Synthetic peptide
NARILLTNY

SEQ ID NO: 478 - protein, 9 aa, Artificial Sequence
  Description of Artificial Sequence Synthetic peptide
NARILFTNY

SEQ ID NO: 479 - protein, 9 aa, Artificial Sequence
  Description of Artificial Sequence Synthetic peptide
NARRLYQQY

SEQ ID NO: 480 - protein, 18 aa, Artificial Sequence
  Description of Artificial Sequence Synthetic peptide
AQRPSSRYASRYLGDYAY

SEQ ID NO: 481 - protein, 9 aa, Artificial Sequence
  Description of Artificial Sequence Synthetic peptide
NARRLFRNY

SEQ ID NO: 482 - protein, 9 aa, Artificial Sequence
  Description of Artificial Sequence Synthetic peptide
HYRRIFNVY

SEQ ID NO: 483 - protein, 11 aa, Artificial Sequence
  Description of Artificial Sequence Synthetic peptide
AAATTSTQWRY

SEQ ID NO: 484 - protein, 9 aa, Artificial Sequence
  Description of Artificial Sequence Synthetic peptide
LARRLFTMY

SEQ ID NO: 485 - protein, 9 aa, Artificial Sequence
  Description of Artificial Sequence Synthetic peptide
NARILLTNY

SEQ ID NO: 486 - protein, 9 aa, Artificial Sequence
  Description of Artificial Sequence Synthetic peptide
NARRLFTNY

SEQ ID NO: 487 - protein, 15 aa, Artificial Sequence
  Description of Artificial Sequence Synthetic peptide
AAATVDWGPRGPYRY

SEQ ID NO: 488 - protein, 20 aa, Artificial Sequence
  Description of Artificial Sequence Synthetic peptide
AAQGVWRGTGSYTWQYSYDY

SEQ ID NO: 489 - protein, 20 aa, Artificial Sequence
  Description of Artificial Sequence Synthetic peptide
ALRIPASSSTTYYYADQYDY

SEQ ID NO: 490 - protein, 9 aa, Artificial Sequence
  Description of Artificial Sequence Synthetic peptide
NARRLFLNY

SEQ ID NO: 491 - protein, 9 aa, Artificial Sequence
  Description of Artificial Sequence Synthetic peptide
NARRLWDIY

SEQ ID NO: 492 - protein, 17 aa, Artificial Sequence
  Description of Artificial Sequence Synthetic peptide
AATKGIVNYRVAGTYDA

SEQ ID NO: 493 - protein, 9 aa, Artificial Sequence
  Description of Artificial Sequence Synthetic peptide
NARRLWDNY

SEQ ID NO: 494 - protein, 9 aa, Artificial Sequence
  Description of Artificial Sequence Synthetic peptide
NARRLWTTY

SEQ ID NO: 495 - protein, 9 aa, Artificial Sequence
  Description of Artificial Sequence Synthetic peptide
NARRLWNAY

SEQ ID NO: 496 - protein, 9 aa, Artificial Sequence
  Description of Artificial Sequence Synthetic peptide
HARRLWDTY

SEQ ID NO: 497 - protein, 10 aa, Artificial Sequence
  Description of Artificial Sequence Synthetic peptide
NARRLWDTYW

SEQ ID NO: 498 - protein, 10 aa, Artificial Sequence
  Description of Artificial Sequence Synthetic peptide
NAVLVRARGM

SEQ ID NO: 499 - protein, 9 aa, Artificial Sequence
  Description of Artificial Sequence Synthetic peptide
FARRLWDIH

SEQ ID NO: 500 - protein, 9 aa, Artificial Sequence
  Description of Artificial Sequence Synthetic peptide
HARRLFRVY

SEQ ID NO: 501 - protein, 9 aa, Artificial Sequence
  Description of Artificial Sequence Synthetic peptide
NARRLLTSM

SEQ ID NO: 502 - protein, 9 aa, Artificial Sequence
  Description of Artificial Sequence Synthetic peptide
NARILLTNY

SEQ ID NO: 503 - protein, 17 aa, Artificial Sequence
  Description of Artificial Sequence Synthetic peptide
AVGITGDGTIQGGPYQY

SEQ ID NO: 504 - protein, 9 aa, Artificial Sequence
  Description of Artificial Sequence Synthetic peptide
NARILLTNY

SEQ ID NO: 505 - protein, 9 aa, Artificial Sequence
  Description of Artificial Sequence Synthetic peptide
NARRLFAIN

SEQ ID NO: 506 - protein, 9 aa, Artificial Sequence
  Description of Artificial Sequence Synthetic peptide
QSRRLFTVY

SEQ ID NO: 507 - protein, 15 aa, Artificial Sequence
  Description of Artificial Sequence Synthetic peptide
AAFSIRGGLRPSYKY

SEQ ID NO: 508 - protein, 12 aa, Artificial Sequence
  Description of Artificial Sequence Synthetic peptide
NLNPADWGRLRN

SEQ ID NO: 509 - protein, 10 aa, Artificial Sequence
  Description of Artificial Sequence Synthetic peptide
NAVLVRARGM

SEQ ID NO: 510 - protein, 17 aa, Artificial Sequence
  Description of Artificial Sequence Synthetic peptide
AAAKRLSIVTLPRQYEF

SEQ ID NO: 511 - protein, 9 aa, Artificial Sequence
  Description of Artificial Sequence Synthetic peptide
SARRIATTY

SEQ ID NO: 512 - protein, 9 aa, Artificial Sequence
  Description of Artificial Sequence Synthetic peptide
NARALWRAY

SEQ ID NO: 513 - protein, 17 aa, Artificial Sequence
  Description of Artificial Sequence Synthetic peptide
AASDDYGLQIKEVAYKY

SEQ ID NO: 514 - protein, 17 aa, Artificial Sequence
  Description of Artificial Sequence Synthetic peptide
AAAKRLSIVALPRQYEF

SEQ ID NO: 515 - protein, 17 aa, Artificial Sequence
  Description of Artificial Sequence Synthetic peptide
AAAKRLSRVTLPREYEF

SEQ ID NO: 516 - protein, 9 aa, Artificial Sequence
  Description of Artificial Sequence Synthetic peptide
NARRLWNAY

SEQ ID NO: 517 - protein, 6 aa, Artificial Sequence
  Description of Artificial Sequence Synthetic 6xHis tag
HHHHHH

(19) United States
(12) Patent Application Publication
(10) Pub. No.: US 2022/0119506 A1
(43) Pub. Date: Apr. 21, 2022

(54) ANTIBODIES AGAINST AQUACULTURE DISEASE-CAUSING AGENTS AND USES THEREOF

(71) Applicant: NOVOBIND LIVESTOCK THERAPEUTICS INC., Vancouver (CA)

(72) Inventors: Hamlet ABNOUSI, Vancouver (CA); Slade LOUTET, Vancouver (CA); Filip VAN PETEGEM, Vancouver (CA); Tsz Ying Sylvia CHEUNG, Vancouver (CA)

(21) Appl. No.: 15/734,939

(22) PCT Filed: Jun. 4, 2019
(86) PCT No.: PCT/IB2019/000687

§ 371 Date: Dec. 3, 2020

Related U.S. Application Data
(60) Provisional application No. 62/680,736, filed on Jun. 5, 2018.

(51) Int. Cl. C07K 16/12; C07K 16/08

(52) U.S. Cl. CPC C07K 16/1239; C07K 2317/565; C07K 2317/56; C07K 16/081

(57) ABSTRACT

Described herein are methods and antibodies useful for reducing, eliminating, or preventing infection with a bacterial or viral population in an aquatic animal. Also described herein are antigens useful for targeting by heavy chain antibodies and VHH fragments for reducing a bacterial or viral population in an aquatic animal.

### CROSS-REFERENCE TO RELATED APPLICATIONS

This application claims the benefit of U.S. Provisional Application No. 62/680,736, filed Jun. 5, 2018, which application is incorporated herein by reference. Priority is claimed pursuant to 35 U.S.C. § 119. The above noted patent application is incorporated by reference as if set forth fully herein.

### FIELD OF THE INVENTION

This invention relates to methods and compositions for the control of microorganisms in aquaculture and uses thereof.

### BACKGROUND OF THE INVENTION

Losses to the aquaculture industry following contamination of livestock with pathogens are a global burden. With a growing global population and no significant increase in the amount of farm land available to agriculture, there is a need to produce larger quantities of food without using more space. Aquaculture is an especially attractive use of this space because the feed conversion ratio for aquaculture organisms is roughly 1:1, whereas the ratio for larger farmed sources of protein is 1:3 or higher⁽¹⁾. Losses to the global aquaculture industry due to pathogens is estimated to be around 40%, or $6 billion USD per annum⁽²⁾. Traditional treatment of animals with antibiotics is a major contributor to the emergence of multi-drug resistant organisms and is widely recognized as an unsustainable solution to controlling contamination of livestock. There is a need for the development of pathogen-specific molecules that inhibit infection or association of the pathogen with the host, without encouraging resistance.

### SUMMARY OF THE INVENTION

With reference to the definitions set out below, described herein are polypeptides comprising heavy chain variable region fragments (V_{H}Hs) whose intended use includes applications in aquaculture, diagnostics, in vitro assays, feed, therapeutics, substrate identification, nutritional supplementation, bioscientific and medical research, and companion diagnostics. Also described herein are polypeptides comprising V_{H}Hs that bind to and decrease the virulence of disease-causing agents in aquaculture. Further to these descriptions, set out below are the uses of polypeptides that comprise V_{H}Hs in methods of reducing transmission and severity of disease in host animals, including their use as an ingredient in a product. Further described are the means to produce, characterize, refine and modify V_{H}Hs for this purpose.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1B: Panel A shows a schematic of camelid heavy chain only antibodies and their relationship to V_{H}H domains. Panel B illustrates the framework regions (FRs) and complementarity determining regions (CDRs) of the V_{H}H domain.

FIGS. 2A-2F: Show phage ELISA binding data for V_{H}H antibodies of this disclosure.

FIG. 3: Shows binding of a selection of recombinantly expressed and purified V_{H}H antibodies to PirA using a protein pull-down assay.

FIG. 4: Shows the stability of a selection of recombinantly expressed and purified V_{H}H antibodies to PirA in shrimp midgut extract fluids.

### DEFINITIONS

In describing the present invention, the following terminology is used in accordance with the definitions below.

In the following description, certain specific details are set forth in order to provide a thorough understanding of various embodiments. However, one skilled in the art will understand that the embodiments provided may be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

### 1) Host

As referred to herein, “host”, “host organism”, “recipient animal”, “host animal” and variations thereof refer to the intended recipient of the product when the product constitutes a feed. In certain embodiments, the host is a crustacean, a shellfish, a shrimp or a prawn.

### 2) Shellfish

As referred to herein, “shellfish” refers to any aquatic exoskeleton-bearing invertebrate. Shellfish can be harvested from the wild or reared. Without limitation, shellfish includes crustaceans, bivalvia, gastropods, cephalopods, octopus, squid, cuttlefish, clams, oysters, mussels, scallops, cockles, whelks, winkles, shrimp, prawns, crawfish, crayfish, lobster, crabs, krill and barnacles.

### 3) Aquaculture-Specific

As referred to herein, “aquaculture”, “aquatic” and variations thereof refer to the cultivation or dwelling of organisms, including animals and plants, in water.

### 4) Pathogens

As referred to herein, “pathogen”, “pathogenic”, and variations thereof refer to virulent microorganisms, that can be associated with host organisms, that give rise to a symptom or set of symptoms in that organism that are not present in uninfected host organisms, including the reduction in ability to survive, thrive, reproduce. Without limitation, pathogens encompass parasites, bacteria, viruses, prions, protists, fungi and algae. In certain embodiments, the pathogen is a bacterium belonging to the Vibrio genus. In certain embodiments, the pathogen is the White Spot Syndrome Virus.

“Virulence”, “virulent” and variations thereof refer to a pathogen's ability to cause symptoms in a host organism. “Virulence factor” refers to nucleic acids, plasmids, genomic islands, genes, peptides, proteins, toxins, lipids, macromolecular machineries or complexes thereof that have a demonstrated or putative role in infection.

“Disease-causing agent” refers to a microorganism, pathogen or virulence factor with a demonstrated or putative role in infection.

### 5) Bacteria

As referred to herein, “bacteria”, “bacterial” and variations thereof refer, without limitation, to Vibrio species, Aeromonas species, Edwarsiella species, Streptococcus species, Rickettsia species, or any other bacterial species associated with aquatic organisms or host organisms. In certain embodiments, bacteria may not be virulent in all host organisms it is associated with.

### 6) Viruses

As referred to herein, “virus”, “viral” and variations thereof refer, without limitation, to the White Spot Syndrome Virus, or any other viral species associated with aquatic organisms or host organisms.

### 7) Antibodies

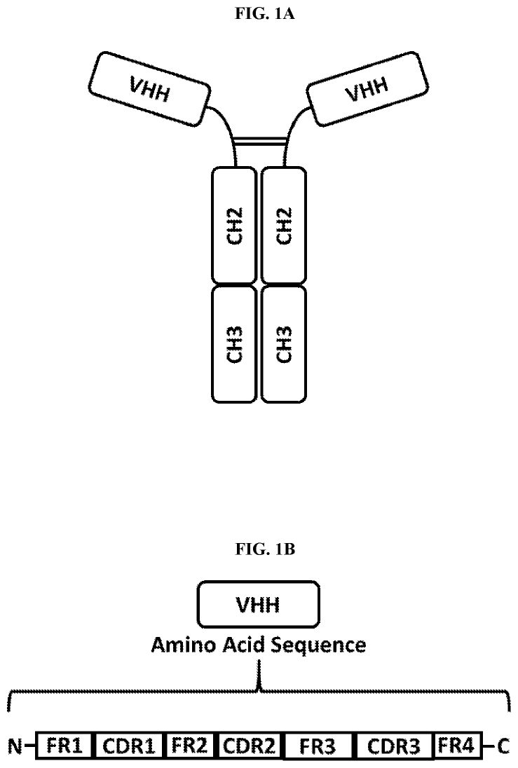

A schematic of camelid heavy chain only antibodies and their relationship to V_{H}H domains and complementarity determining regions (CDRs) is shown in FIG. 1. (Panel A). A camelid heavy chain only antibody consists of two heavy chains linked by a disulphide bridge. Each heavy chain contains two constant immunoglobulin domains (CH2 and CH3) linked through a hinge region to a variable immunoglobulin domain (V_{H}H). (Panel B) are derived from single V_{H}H domains. Each V_{H}H domain contains an amino acid sequence of approximately 110-130 amino acids. The V_{H}H domain consists of the following regions starting at the N-terminus (N): framework region 1 (FR1), complementarity-determining region 1 (CDR1), framework region 2 (FR2), complementarity-determining region 2 (CDR2), framework region 3 (FR3), complementarity-determining region 3 (CDR3), and framework region 4 (FR4). The domain ends at the C-terminus (C). The complementarity-determining regions are highly variable, determine antigen binding by the antibody, and are held together in a scaffold by the framework regions of the V_{H}H domain. The framework regions consist of more conserved amino acid sequences; however, some variability exists in these regions.

As referred to herein “V_{H}H” refers to an antibody or antibody fragment comprising a single heavy chain variable region which may be derived from natural or synthetic sources. NBXs referred to herein are an example of a V_{H}H. In a certain aspect a V_{H}H may lack a portion of a heavy chain constant region (CH2 or CH3), or an entire heavy chain constant region.

As referred to herein “heavy chain antibody” refers to an antibody that comprises two heavy chains, and lacking the two light chains normally found in a conventional antibody. The heavy chain antibody may originate from a species of the Camelidae family or Chondrichthyes class. Heavy chain antibodies retain specific binding to an antigen in the absence of any light chain

As referred to herein “specific binding”, “specifically binds” or variations thereof refer to binding that occurs between an antibody and its target molecule that is mediated by at least one complementarity determining region (CDR) of the antibody's variable region. Binding that is between the constant region and another molecule, such as Protein A or G, for example, does not constitute specific binding.

As referred to herein “antibody fragment” refers to any portion of a conventional or heavy chain antibody that retains a capacity to specifically bind a target antigen and may include a single chain antibody, a variable region fragment of a heavy chain antibody, a nanobody, a polypeptide or an immunoglobulin new antigen receptor (IgNAR).

As referred to herein an “antibody originates from a species” when any of the CDR regions of the antibody were raised in an animal of said species. Antibodies that are raised in a certain species and then optimized by an in vitro method (e.g., phage display) are considered to have originated from that species.

As referred to herein “conventional antibody” refers to any full-sized immunoglobulin that comprises two heavy chain molecules and two light chain molecules joined together by a disulfide bond. In certain embodiments, the antibodies, compositions, feeds, products, and methods described herein do not utilize conventional antibodies.

### 8) Production System

As referred to herein, “production system” and variations thereof refer to any system that can be used to produce any physical embodiment of the invention or modified forms of the invention. Without limitation, this includes but is not limited to biological production by any of the following: bacteria, yeast, algae, arthropods, arthropod cells, plants, mammalian cells. Without limitation, biological production can give rise to antibodies that can be intracellular, periplasmic, membrane-associated, secreted, or phage-associated. Without limitation, “production system” and variations thereof also include, without limitation, any synthetic production system. This includes, without limitation, de novo protein synthesis, protein synthesis in the presence of cell extracts, protein synthesis in the presence of purified enzymes, and any other alternative protein synthesis system.

### 9) Product

As referred to herein, “product” refers to any physical embodiment of the invention or modified forms of the invention, wherein the binding of the V_{H}H to any molecule, including itself, defines its use. Without limitation, this includes a feed, a feed additive, a nutritional supplement, a premix, a medicine, a therapeutic, a drug, a diagnostic tool, a component or entirety of an in vitro assay, a component or the entirety of a diagnostic assay (including companion diagnostic assays).

### 10) Feed Product

As referred to herein, “feed product” refers to any physical embodiment of the invention or modified forms of the invention, wherein the binding of the V_{H}H to any molecule, including itself, defines its intended use as a product that is taken up by a host organism. Without limitation, this includes a feed, a pellet, a feed additive, a nutritional supplement, a premix, a medicine, a therapeutic or a drug.

### DETAILED DESCRIPTION OF THE INVENTION

Descriptions of the invention provided are to be interpreted in conjunction with the definitions and caveats provided herein.

Some farmed aquatic organisms, such as some crustaceans, lack a true adaptive immune response. Additionally, the administration of therapeutics by injection for small and intensely reared organisms is cumbersome. For these reasons, vaccine-based approaches to protecting farmed aquaculture organisms from pathogenic infection is ineffective. Secondly, the use of antibiotics as growth promoters in animal feed has already been banned in Europe (effective from 2006) in an effort to phase out antibiotics for non-medicinal purposes and limit antimicrobial resistance. Indeed, many bacterial pathogens of aquatic organisms already harbor resistance to common antibiotics. This underpins the need for the development of non-antibiotic products to administer to aquatic organisms to prevent infection and promote growth.

Significant pathogens affecting farmed aquatic organisms include bacteria, such as members of the Vibrio genus, among others, as well as viruses such as White Spot Syndrome Virus (WSSV). Losses due to Vibrio parahaemolyticus, for example, first emerged in 2009 and have been prevalent ever since⁽³⁾. It was not until 2013 that V. parahaemolyticus was shown to be the causative agent of Acute Hepatopancreatic Necrosis Disease (AHPND): a subtype of Early Mortality Syndrome (EMS) that contributes approximately $1 billion USD loss to the shrimp farming industry per annum^{(4, 5)}. In 2015 it was demonstrated that the presence of the pVA-1 plasmid and the toxins encoded (PirA and PirB) are directly responsible for AHPND⁽⁵⁾. Once infected, organisms are up to 100% moribund within 3 days. V. parahaemolyticus is also a prevalent human pathogen, responsible for gastrointestinal infection and septicemia after exposure to contaminated fish or fisheries⁽⁶⁾. In addition to PirA and PirB, V. parahaemolyticus produces several proteinaceous factors that have been demonstrated to facilitate host infection and can be targeted to curb virulence.

WSSV infection is a longer-standing problem; having been identified in 1992⁽⁷⁾ there is still no effective means of controlling viral spread or infection in aquatic organisms. Cumulative losses to the aquaculture industry as a consequence of WSSV are estimated at $15 billion USD⁽⁸⁾. Infected organisms are moribund within 3-5 days. The surface of the viral envelope is well characterized and can be targeted to prevent infection.

Other disease-causing agents affecting farmed aquaculture organisms include bacteria (such as Yersinia spp., Edwarsiella spp., Aeromonas spp., Streptococcus spp. and Rickettsia spp.), viruses (such as White Spot Syndrome Virus (WSSV), Yellowhead virus, tilapia iridovirus, epizootic hematopoietic necrosis virus (EHNV), infectious hematopoietic necrosis virus (IHNV), infectious salmon anemia virus (ISAV), infectious pancreatic necrosis virus (IPNV), infectious hypodermal and hematopoietic necrosis virus (IHHNV), taura syndrome virus (TSV) and white spot bacilloform virus (WSBV), hepatopancreatic parvo-like virus (HPV), reo-like virus, monodon baculovirus (MBV), baculoviral midgut GI and necrosis virus (BMN)), algae, prions, protists, parasites, fungi, peptides, proteins and nucleic acids. To our knowledge, an effective, non-vaccine-based treatment against any of these disease-causing agents has yet to be developed for commercial use.

Existing methods fail to acknowledge the limited immune development of aquatic organisms affected by the pathogens listed above, and as such rely on the host organism to generate protection against disease-causing agents. This approach is limited by the inadequacies of the host organism's immune system and therefore does not provide an effective means of protection. This problem is circumvented by introducing exogenous peptides into the host that neutralize the virulence and spread of the disease-causing agent without eliciting the host immune response. Moreover, the methods described herein provide scope for the adaptation and refinement of neutralizing peptides, which provides synthetic functionality beyond what the host is naturally able to produce.

Antibody heavy chain variable region fragments (V_{H}Hs) are small (12-15 kDa) proteins that comprise specific binding regions to antigens. When introduced into an animal, V_{H}Hs bind and neutralize the effect of disease-causing agents in situ. Owing to their smaller mass, they are less susceptible than conventional antibodies, such as previously documented IgYs, to cleavage by enzymes found in host organisms, more resilient to temperature and pH changes, more soluble, have low systemic absorption and are easier to recombinantly produce on a large scale, making them more suitable for use in animal therapeutics than conventional antibodies.

### Antibodies for Preventing or Reducing Virulence (Summary)

In one aspect, the present invention provides a polypeptide or pluralities thereof comprising a V_{H}H or V_{H}Hs that bind disease-causing agents to reduce the severity and transmission of disease between and across species. In certain embodiments, the V_{H}H is supplied to host animals. In certain embodiments, the V_{H}H is an ingredient of a product.

### Binding to Reduce Virulence

In another aspect, the present invention provides a polypeptide or pluralities thereof comprising a V_{H}H or V_{H}Hs that bind disease-causing agents, and in doing so, reduce the ability of the disease-causing agent to exert a pathological function or contribute to a disease phenotype. In certain embodiments, binding of the V_{H}H(s) to the disease-causing agent reduces the rate of replication of the disease-causing agent. In certain embodiments, binding of the V_{H}H(s) to the disease-causing agent reduces the ability of the disease-causing agent to bind to its cognate receptor. In certain embodiments, binding of the V_{H}H(s) to the disease-causing agent reduces the ability of the disease-causing agent to interact with another molecule or molecules. In certain embodiments, binding of the V_{H}H(s) to the disease-causing agent reduces the mobility or motility of the disease-causing agent. In certain embodiments, binding of the V_{H}H(s) to the disease-causing agent reduces the ability of the disease-causing agent to reach the site of infection. In certain embodiments, binding of the V_{H}H(s) to the disease-causing agent reduces the ability of the disease-causing agent to cause cell death.

Antibodies Derived from Llamas

In a further aspect, the present invention provides a method for the inoculation of Camelid or other species with recombinant virulence factors, the retrieval of mRNA encoding V_{H}H domains from lymphocytes of the inoculated organism, the reverse transcription of mRNA encoding V_{H}H domains to produce cDNA, the cloning of cDNA into a suitable vector and the recombinant expression of the V_{H}H from the vector. In certain embodiments, the camelid can be a dromedary, camel, llama, alpaca, vicuna or guanaco, without limitation. In certain embodiments, the inoculated species can be, without limitation, any organism that can produce single domain antibodies, including cartilaginous fish, such as a member of the Chondrichthyes class of organisms, which includes for example sharks, rays, skates and sawfish. In certain embodiments, the heavy chain antibody comprises a sequence set forth in Table 1. In certain embodiments, the heavy chain antibody comprises an amino acid sequence with at least 80%, 90%, 95%, 97%, 99%, or 100% identity to any sequence disclosed in Table 1. In certain embodiments, the heavy chain antibody possesses a CDR1 set forth in Table 2. In certain embodiments, the heavy chain antibody possesses a CDR2 set forth in Table 2. In certain embodiments, the heavy chain antibody possesses a CDR3 set forth in Table 2.

TABLE 1
Unique SEQ IDs for V_{H}H antibodies of this disclosure

| SEQ ID | NBX | Amino acid sequence | Antigen |
|---|---|---|---|
| 1 | NBX0401 | QVQLQQSGGGLVRAGGSLRLSCETSGRTFSSYTMG | PirA |
| | | WFRQAPGKEREFVGTIDWWSSSSSYADSVKGRFTI | |
| | | SRDNAKNTVYLQMNSLKPEDTAVYYCAASGKYGLA | |
| | | YSRRDYAYWGQGTQVTVSS | |
| 2 | NBX0402 | QVQLQQSGGSLVQAGGSLRLSCAASGLPFINYAMG | PirA |
| | | WFRQAPGKDREIVAAIDWNGDSTYYAVSVKGRFTI | |
| | | SRDNAKNTVTLQMNSLKPEDTAIYYCASHYQPYIR | |
| | | VSATRRFEADYWGQGTQVTVSS | |
| 3 | NBX0403 | QVQLQQSGGSLVQAGGSLRLSCAASGLPFINYAMG | PirA |
| | | WFRQAPGKDREIVAAIDWNGDSTYYAVSVKGRFTI | |
| | | SRDNAKNTVTLQMNSLKPEDTAIYYCAADYQPYIR | |
| | | VSATRRFEADYWGQGTQVTVSS | |
| 4 | NBX0404 | QVQLQESGGGLVQAGDSLRLSCATSGRTFSRYTMG | PirB |
| | | WFRQTPGKEREFVAAISWSGTYYTDSVKGRFTISV | |
| | | DNAKNTVYLQMNSLKPEDTAVYYCASGSRRLYYSS | |
| | | DIDYWGQGTQVTVSS | |
| 5 | NBX0405 | QVQLQESGGGLVQAGDSLRLSCATSGRTFSRYTMG | PirB |
| | | WFRQTPGKEREFVAAISWSGTYYTDSVKGRFTISR | |
| | | DNAKNTVYLQMNSLKPEDTAVYYCVVGSRRLYYSS | |
| | | DINYWGQGTQVTVSS | |
| 6 | NBX0406 | QVQLQESGGGLVQAGESLRLSCAASGFTFSTYTWD | VP24 |
| | | WYRQAPGKQREMVARISRDGITNYADSVKGRFTIS | |
| | | RDNAKNTVDLQMNSLKPEDTAVYYCAVVKEDNRYW | |
| | | CHADRNLYRNWGQGTQITVSS | |
| 29 | NBX0601 | QVQLQQSGGGLVQPGGSLRLSCAGSRFTFSTYPMS | PirA |
| | | WVRQAPGKGVEWVSSISVGGGIKNYADSVKGRFTI | |
| | | SRDNAKNTMYLQMNGLKPEDTAVYYCAKGGKTSYT | |
| | | REWGQGTQVTVSS | |
| 30 | NBX0602 | QVQLQESGGGLVQAGDSLRLSCAASGRTFSRYAMG | PirA |
| | | WFRQAPGKEREFVAAVDWSGGSTAYADSVKGRFTI | |
| | | SRDNAKNTVYLQMNSLKPDDTAVYYCAARARDVYG | |
| | | RAWYVEDSSTYDYWGQGTQVTVSS | |
| 31 | NBX0603 | QVQLQESGGGLVQAGGSLRLSCAASGSMFSINAMG | PirA |
| | | WYRQAPGNEREWVATISRGGITYYDDSVKGRFTIS | |
| | | RDNAKNTVYLQMNSLKPEDTAVYFCNAENRRLGDD | |
| | | FWGQGTQVTVSS | |
| 32 | NBX0604 | QVQLQESGGGLVQAGGSLRLSCAASGRTFSSYTMG | PirA |
| | | WFRQVPGKEREFVAAIRWSGGSRIYADSVKGRFTI | |
| | | SRDNTKNVVYLQMTSLKPEDTAVYYCAADRDGYSS | |
| | | YAHQYDYWGQGTQVTVSS | |
| 33 | NBX0605 | QVQLQESGGGLVQPGGALRLSCAASGSFFSIYAMA | PirA |
| | | WYRQAPGKQRELVAGITSGSETNYADSVKGRFTIS | |
| | | RDNAKNTVYLQMNSLKLEDTAVYYCNRWAPLTRLD | |
| | | YWGQGTQVTVSS | |
| 34 | NBX0606 | QVQLQESGGGLVQAGDSLRLSCAASGRTSSSFAMG | PirA |
| | | WFRQAPGKEREFVGGITRTGGRTYYVDSVKGRFTI | |
| | | SRDNAKNTMSLQMNSLKPEDTAVYYCAARWATATS | |
| | | NSIRVYYNEGQYDYWGQGTQVTVSS | |
| 35 | NBX0607 | QVQLQESGGGLVQAGGSLRLSCAASGGTFSRLTMG | PirA |
| | | WFRQAPGEEREFVAAVSWVAETTDYADSVKGRFSI | |
| | | SRDNSKNTVYLQMNSLKPVDTAVYYCAAGPRDMIR | |
| | | SRNIRSYDSWGQGTQVTVSS | |
| 36 | NBX0608 | QVQLQESGGGLVQAGGSLRLSCAASGSMFNINAMG | PirA |
| | | WYRQAPGKQREWVATISSGGITYYDDSVKGRFTIS | |
| | | RDNAKNTVYLQMNSLKPEDTAVYFCNAENRRLGDD | |
| | | YWGQGTQVTVSS | |
| 37 | NBX0609 | QVQLQESGGGLVQAGGSLRLSCAASGSIFSGNVMG | PirA |
| | | WYRQVPGKLREWVATISSGGITYYDDSVKGRFTIS | |
| | | RDNAKNTVYLQMNSLKPEDTAVYFCNLENRRLGDD | |
| | | YWGQGTQVTVSS | |
| 38 | NBX0610 | QVQLQESGGGLVQAGGSLRLSCAASGSIFSRDAMG | PirA |
| | | WYRQAPGNLREWVATISSGGITYYDDSVKGRFTIS | |
| | | RDNAKNTVYLQMNSVKPEDTAVYFCNAENRRLGDD | |
| | | YWGQGTQVTVSS | |
| 39 | NBX0611 | QVQLQQSGGGLVQAGGSLRLSCAASGGTFSRLTMG | PirA |
| | | WFRQAPGEEREFVAAVSWVAETTDYADSVKGRFSI | |
| | | SRDNSKNTVYLQMNSLKPVDTAVYYCAAGPRDMIR | |
| | | SRNIKSYDSWGQGTQVTVSS | |
| 40 | NBX0612 | QVQLQESGGGLVQAGGSLRLSCVASGTIFSINKMG | PirA |
| | | WYRQAPEKERELVAVARSGGIINYADSVKGRFTIS | |
| | | RDDAKNTVYLQMNSLRPDDTAVYFCNALIHTRYDR | |
| | | VTGYWGQGTQVTVSS | |
| 41 | NBX0613 | QVQLQESGGGLVQAGGSLRLSCAASGSIFSINAMG | PirA |
| | | WYRQAPGKLREWVATISSGGITYYDDSVKGRFTIS | |
| | | RDNAKNTVYLQMNSLKPEDTAVYFCNAENPRLGDD | |
| | | YWGQGTQVTVSS | |
| 42 | NBX0614 | QVQLQQSGGGLVQAGGSLRLSCAASGSSFRSNAIG | PirB |
| | | WYRQFPGKSRELIAVITRSGSTQYADSVKGRFTAS | |
| | | RDNAKNMIYLQMNNLKLEDTAVYYCHDETMKLISV | |
| | | KNDYWGQGSQVTVSS | |
| 43 | NBX0615 | QVQLQESGGGLVQAGGSLRLSCAASGSMFSRNAMG | PirA |
| | | WYRQAPGKQREWVATISSGGITYYDDSVKGRFTIS | |
| | | RDNAKNTVYLQMNSLKPEDTAVYFCNAENRRLGDD | |
| | | YWGQGTQVTVSS | |
| 44 | NBX0616 | QVQLQESGGGLVQAGGSLRLSCATSGLTFSSYAMG | PirA |
| | | WFRQAPGKEREFVATISWSGKSTRYSDSVKGRFTI | |
| | | SRDNAKNTVYLQMNSLKPEDTAVYYCAADYQRLGL | |
| | | LRVGVAEYDYWGQGIQVTVSS | |
| 45 | NBX0617 | QVQLQQSGGGLVQAGGSLRLSCQASGRSGSTSFMG | PirA |
| | | WFRQAPGKEREFVAAIRWSSGMTYYADSVKGRFTI | |
| | | SRDNAKNTVDLQMNSLKPEDTAVYYCAADNYPLHI | |
| | | GHQDHEVDYWGQGTQVTVSS | |
| 46 | NBX0618 | QVQLQESGGGLVQPGGSLRLSCAASGSIFSFNAMG | PirA |
| | | WYRQAPGKQRELVAAITKGGSTSYADSVKGRFTIS | |
| | | VDNAKNTVYLQMNSLTPEDTAVYYCNVKTLRSALF | |
| | | PGYEYWGQGTQVTVSS | |
| 47 | NBX0619 | QVQLQQSGGGLVQAGGSLRLFCAASGGTFSRLTMG | PirA |
| | | WFRQAPGEEREFVAAVSWVAETTDYADSVKGRFSI | |
| | | SRDNSKNTVYLQMNNLKPVDTAVYYCAAGPRDMIR | |
| | | SRNIRSYDSWGQGTQVTVSS | |
| 48 | NBX0620 | QVQLQESGGGLVQAGGSLRLSCAASGGTFSRLTLG | PirA |
| | | WFRQAPGEEREFVAAVSWVAEMTDYADSVKGRFSI | |
| | | SRDNSKNTVYLQMNSLKPVDTAVYYCAAGPRDMVR | |
| | | SRNIRSYDSWGQGTQVTVSS | |
| 49 | NBX0621 | QVQLQESGGGLVQAGGSLRLSCAASGRTFSTYSMG | PirA |
| | | WFRQVPGKEREFVAAIRWSGGSRTYADSVKGRFTI | |
| | | SRDNTKNVVYLQMTSLKPEDTAVYYCAADRDGYSS | |
| | | YAHQYDYWGQGTQVTVSS | |
| 50 | NBX0622 | QVQLQESGGGLVQAGGSLRLSCAASGRLFNINAMG | PirA |
| | | WYRQAPGKQREWVATISSGGITYYDDSVKGRFTIS | |
| | | RDNAKNTVYLQMDSLKPGDTAVYFCNAENRGLGDD | |
| | | YWGQGTQVTVSS | |
| 51 | NBX0623 | QVQLQESGGGWVQTGGSLRLSCAASGRTLSNYAMG | PirA |
| | | WFRQAPGKEREFVAAISRSGMSTDAPNSVKGRFTV | |
| | | SRDNAKNTMYLHLNSLKPEDTAVYYCAARGGLPNP | |
| | | SRTYGFEEQYDYWGQGTQVTVSS | |
| 52 | NBX0624 | QVQLQESGGGLVQAGGSLRLSCAASGRTVSSLPMG | PirA |
| | | WFRQAPGKEREFVAALNWSGTSTYYEDSVKGRFTI | |
| | | SRDNAKNTLYLQMNNLKPEDTAVYYCAAKGAIYYS | |
| | | YSPRNNNNYDVWGQGTQVTVSS | |
| 53 | NBX0625 | QVQLQESGGGLVQAGGSLRLSCAASGSMFNINAMG | PirA |
| | | WYRQAPGKQREWVATISSGGITYYDDSVKGRFTIS | |
| | | RDNAKNTVHLQMNSLKPEDTAVYFCNAENRLGDDY | |
| | | WGQGTQVTVSS | |
| 54 | NBX0626 | QVQLQQSGGGLVQAGGSLRLACAVSETTLATNAMA | PirB |
| | | WYRQAQGKRREWVATISSVSSGGITNYSGSVKGRF | |
| | | TISRDNAKNTVFLQMNSLQPEDTAVYYCNGVRRGR | |
| | | SYWGQGTQVTVSS | |
| 55 | NBX0627 | QVQLQQSGGGLVQAGGSLRLSCAASGSSFRSNAIG | PirB |
| | | WYRQSPGKSRELIAVITRSGSTQYADSVKGRFTAS | |
| | | RDNAKNMIYLQMNSLKPEDTAVYYCHDETMKLITG | |
| | | KNDYWGQGTQVTVSS | |
| 56 | NBX0628 | QVQLQQSGGGLVQVGGSLRLSCAASGRTFSSYAMG | PirA |
| | | WFRQVPGKEREFVAAIKWSGGSRTYADSVAGRFTI | |
| | | SRDNTKNWYLQMTSLKPEDTAVYYCAADRDGYSRY | |
| | | AHQYDYWGQGTQVTVSS | |
| 57 | NBX0629 | QVQLQQSGGGLVQAGGSLRLSCAASGGTFSRLTIG | PirA |
| | | WFRQAPGEERVFVAAVSWVAETTDYADSVKGRFSI | |
| | | SRDNSKNTVYLQMNSLKPVDTAVYYCAAGPRDMIR | |
| | | SRNIRSYDSWGQGTQVTVSS | |
| 58 | NBX0630 | QVQLQQSGGGLVPGGGSLRLSCAASGVTFSDYPMA | PirA |
| | | WYRTAPGKQRELVASISAGGGLIKYVDSVKGRFTI | |
| | | SRDNAKNTLYLQMNSLKPEDTGVYLCNLKTSYFWP | |
| | | WGQGTQVTVSS | |
| 59 | NBX0631 | QVQLQESGGGLVQAGDSLRLSCKASGGTFSRLTIA | PirA |
| | | WFRQAPGKEREFVTAVSWVAQTTDYADSVKGRFSI | |
| | | SRDNSKNTVYLQMNSLKPLDTAVYYCAAGPQDMIR | |
| | | SRNIRSYISWGQGTQVTVSS | |
| 60 | NBX0632 | QVQLQQSGGGLVQAGGSLRLSCAASGGTFSRLTMG | PirA |
| | | WFRQAPGEEREFVAAVSWVAGTTDYADSVKGRFTI | |
| | | SRDNSKNTVHLQMNSLKPVDTAVYYCAAGPRDMIR | |
| | | SRNIRSYDSWGQGTQVTVSS | |
| 61 | NBX0633 | QVQLQESGGGLVQPGGSLRLSCTASGTIFRSKSMA | PirA |
| | | WYRQAPGQGRETVAHISGLGHTNYVESVKGRFTVS | |
| | | RDDAKNAVYLQMSSLKPEDTAVYYCNTFTAAFSWG | |
| | | QGTQVTVSS | |
| 62 | NBX0634 | QVQLQQSGGGLVQAGGSLRLSCAASGGSFSRLTLG | PirA |
| | | WFRQAPGEEREFVAAVSWVAETTDYADSVKGRFSI | |
| | | SRDNSKNTVYLQMNSLKPVDTAVYYCAAGPRDMIR | |
| | | SRNIRSYASWGQGTQVTVSS | |
| 63 | NBX0635 | QVQLQESGGGLVQAGGSLRLSCATSGLTFSNYAMG | PirA |
| | | WFRQAAGKEREFVATISWSGKSTRYADSVKGRFTI | |
| | | SRDNAKNTVDLRMNSLKPEDTAVYYCAAEYQRLGL | |
| | | LRDGVADYSYWGQGTQVTVSS | |
| 64 | NBX0636 | QVQLQESGGGLVQAGGSLRLSCAASGGTFSRLTMG | PirA |
| | | WFRQAPGEEREFVAAVSWVAETTDYADSVKGRFSI | |
| | | SRDNSKNTVYLQMNSLKPVDTAVYYCAAGPRDMIR | |
| | | SRNIRSYVSWGQGTQVTVSS | |
| 65 | NBX0637 | QVQLQESGGGLVQAGNSLKLSCVASGRTFSSYPMG | PirA |
| | | WYRTAPGKQRELVASISAGGGLIKYVDSVKGRFTI | |
| | | SRDNAKNTLYLQMNSLKPEDTGVYLCNLKTSYFWP | |
| | | WGQGTQVTVSS | |
| 66 | NBX0638 | QVQLQESGGGLVQAGGSLRLSCAASGSIFSGNVMG | PirB |
| | | WYRQVPGKQRDLVATITGGGITRYADSVKGRFTIS | |
| | | RDNAKNTVYLQMNSLKPEDTAVYYCNYRRIMQQYW | |
| | | GKGTLVTVSS | |
| 67 | NBX0639 | QVQLQESGGGLVQAGGSLRLSCAASGIVFSSIVMA | PirB |
| | | WYRQAPGKQRELVASITNGGLVNSGDSVKGRFTIS | |
| | | RDNAKNTVYLQMNSLKPEDTAVYYCNARRIMTSYW | |
| | | GQGTQVTVSS | |
| 68 | NBX0640 | QVQLQESGGGLVQPGGSLRLSCAASGSIFSGNVMG | PirB |
| | | WYRQVPGKQRDLVATITGGGITRYADSVKGRFTIS | |
| | | RDNAKNTVYLQMNSLKPEDTAVYYCHYRRIMQQYW | |
| | | GQGTQVTVSS | |
| 69 | NBX0641 | QVQLQESGGGLVQAGGSLRLSCAASGSISNSYVMG | PirB |
| | | WYRQAPGKQRELVATITSGGLTNYAQSLKGRFTIS | |
| | | RDNAKNTVYLQMTSLEPEDTAVYYCNARVIFTTYW | |
| | | GQGTQVTVST | |
| 70 | NBX0642 | QVQLQQSGGGLVQAGGSLRLSCVASTVTFSRYAMG | PirB |
| | | WFRQAPGKEREVVAGISGSGHRTYYGDFVKGRFTI | |
| | | SRDNAKKTVYLQMNNLKPEDTAVYYCAGDLVAKFD | |
| | | SAYRVSYDSWGQGTQVTVSS | |
| 71 | NBX0643 | QVQLQESGGGLVQAGGSLRLSCEASGSIFSGNVMG | PirB |
| | | WYRQVPGKQRDLVATMTGGGVTRYADSVKARFTIS | |
| | | RDNAKNTVYLQMNSLKPEDTGVYYCHYRRIMQQYW | |
| | | GQGTQVTVSS | |
| 72 | NBX0644 | QVQLQESGGGLVQAGGSLRLSCAASGSIFSGNVMG | PirB |
| | | WYRQVPGKQRDLVATITGGGITRYADSVKGRFTIS | |
| | | RDNAKNKVYLQMNGLKPEDTAVYFCFYRRIMQQYW | |
| | | GQGTQVTVSS | |
| 73 | NBX0645 | QVQLQESGGGLVQAGGSLRLSCAASGSIFSGNVMG | PirB |
| | | WYRQVPGKQRDLVATITGGGITHYADSVKGRFTIS | |
| | | RDNAKNTVYLQMNSLKPEDTAVYYCLYRRIMQQYW | |
| | | GQGTQVTVSS | |
| 74 | NBX0646 | QVQLQESGGGLVQAGGSLRLSCAASGSIFSGNVMG | PirB |
| | | WYRQVPGNQRELVATITGGGVTRYADSVKARFTIS | |
| | | RDNAKNTVYLQMNSLKPEDTAVYYCHYRRIMQQSW | |
| | | GQGTQVTVSS | |
| 75 | NBX0647 | QVQLQESGGGSVPPGGSLRLSCAASGSIFSGNVMA | PirB |
| | | WYRQVPGKQRDLVASMTGGGVTRYADSVIARFTIS | |
| | | RDNVKNTVYLQMNSLKPEDTAVYYCHYRRIMQQYW | |
| | | GQGTQVTVSS | |
| 76 | NBX0648 | QVQLQESGGGLVQAGGSLRLSCEASGIIFSSNVMG | PirB |
| | | WYRQAPGKQRELVASRTSGGLTNYADSAKGRFTIS | |
| | | RDNAKNTVYLQMNSLKPEDTAVYYCNARRLFTNYW | |
| | | GQGTQVTVSS | |
| 77 | NBX0649 | QVQLQQSGGGLVQPGGSLTLSCAASGSIASGNVLG | PirB |
| | | WYRQAPGKQRELVATITSGGLTHYKDSVKGRFTIS | |
| | | RDNAKNMVFLQMNSLKPEDTAVYYCNYRRLATGYW | |
| | | GQGTQVTVSS | |
| 78 | NBX0650 | QVQLQESGGGLVQAGGSLRLSCEASGSIFSGNVLG | PirB |
| | | WYRQVPGKQRDLVATITGGGITRYADSVKGRFTIS | |
| | | RDNAKNTVYLQMNALKPEDTAVYYCHYRRIMQQYW | |
| | | GQGTQVTVSS | |
| 79 | NBX0722 | QVQLQESGGGLVQAGGSLRLSCRASGRTFSSYPMG | VP28 |
| | | WFRQAPGKEREQIAGISRSGDPGKYAASVSGRFTI | |
| | | SRDNAKNTLYLQMNSLKPEDTAVYYCAARQIYSNN | |
| | | YSYWGQGTQVTVSS | |
| 80 | NBX0723 | QVQLQESGGGLVQAGGSLRLSCAASGRTFSSYPMG | VP28 |
| | | WFRQAPGSEREQIAGISRSGVPGKYADSVSGRFTI | |
| | | SRDNAKNTLYLQMNSLKPEDTAVYYCAARSIYSNN | |
| | | YSYWGQGTQVTVSS | |
| 81 | NBX0724 | QVQLQQSGGGLVQAGGSLRLSCAASGRTFSSYPMG | VP28 |
| | | WFRQAPGSEREQIAGISRSGVSGKYADSVSGRFTI | |
| | | SRDNAKNTLYLQMNSLKPEDTAVYYCAARSIYSNN | |
| | | YSYWGQGTQVTVSS | |
| 82 | NBX0725 | QVQLQQSGGGLVQAGGSLRLSCTASGRTFSSYPMG | VP28 |
| | | WFRQAPGKEREQIAGISRSGNPGKYADSVSGRFTI | |
| | | SRDNAKNTLYLQMNSLKPEDTAVYYCAARQIYSNN | |
| | | YSYWGQGTQVTVSS | |
| 83 | NBX0730 | QVQLQESGGGLVQTGDSLRLACAASGRTFSSYPMA | VP28 |
| | | WYRQAPGQEREFVAGINRNGNIPVYADSVKGRFTI | |
| | | SRDNAKNTVYLQMNNLKPEDTAVYYCAARTIYDSH | |
| | | YTSWGQGTQVTVSS | |
| 84 | NBX0737 | QVQLQQSGPGLVKPSETLSLTCTVSDGAITGSYYV | VP24 |
| | | WSWIRQPPGKGLEWMGVITYDGSTYYNPSLESRTS | |
| | | ISRDTSKNQFSLQLDSVTREDTAVYYCARAGEEYI | |
| | | CSGYGCHGSLGLDYWGKGTLVTVSS | |
| 85 | NBX0738 | QVQLQESGGGLVQPGGSLGLSCAASGFTFGSYAMS | VP24 |
| | | WVRQAPGKGPEWVSGENSGDGRITYADSVKGRFTI | |
| | | SRDNTKNTLYLQMNSLKPEDTAVYYCATGIRTPII | |
| | | WGQGTQVTVSS | |
| 86 | NBX0739 | QVQLQESGGGLVQAGGSLRLSCAASGSIFTSDVGW | VP24 |
| | | NRQAPGSVREVVARMTSAGTTIYGDDVMGRFTISR | |
| | | DNAKSTVYLQMNSLLPEDTGVYYCGVGRFWGQGTQ | |
| | | VTVSS | |
| 87 | NBX0745 | QVQLQESGGGLVQAGGSLRLSCAASGRTFSSYPMG | VP28 |
| | | WFRQAPGKEREQIAGISRSGDPGKYAASVSGRFTI | |
| | | SRDNAKNTLYLQMNSLKPEDTAVYYCAAREIYSNN | |
| | | YSYWGQGTQVTVSS | |
| 88 | NBX0746 | QVQLQESGGGLVQAGGSLRLSCAASGRTFSSYAMG | VP28 |
| | | WFRRAPGKEREQIAGISRSGNPGKYADSVSGRFTI | |
| | | SRDNAKNTLYLQMNSLKPEDTAVYYCAARQIYSNN | |
| | | YSYWGQGTQVTVSS | |
| 89 | NBX0813 | QVQLQESGGGLVQAGGSLRLSCVVSGMLFSIRNMR | PirA |
| | | WYRQAPGKQRELVAQIGSSGNTDYVESVKGRFTIS | |
| | | RDNAKNTVYLQMNSLKPEDTAVYFCNALNYWGKGT | |
| | | LVTVSS | |
| 90 | NBX0814 | QVQLQQSGGDLVQAGGSLRLSCAASMRTFNSRTIG | PirA |
| | | WFRQAPGKGRELAAAIAWTGGNTYYADSVKGRFTI | |
| | | SRDNAKNTVYLQMNSLKPEDTAVYYCAAQTRPYDL | |
| | | PSIRPDDYASRGQGTQVTVSS | |
| 91 | NBX0815 | QVQLQESGGGLVQAGGSLRLSCAASGRTFSSYAMG | PirA |
| | | WFRQSPGKDREFVAAVSWSGGSTYYADSLKGRFTI | |
| | | SRDNAKNTVYLQMNSLKPEDTADYYCAAQRVMDYY | |
| | | RPRTESAYAYWGQGTRVTVSS | |
| 92 | NBX0816 | QVQLQESGGGLVQAGGSLRLSCAASEYIFSNFGMG | PirA |
| | | WFRQAPGKEREFVGAISRSGSRMSYADSVKGRFII | |
| | | SRDNTKNTVYLQMNSLKPEDTAVYYCAAVYGQYSY | |
| | | HYSSDSKQYSYWGQGTQVTVSS | |
| 93 | NBX0817 | QVQLQESGGGLVQAGGSLRLSCGASGGTNSNYAMG | PirA |
| | | WFRQPPGKKREFVAALSWSGYNTHYADSVKGRFTI | |
| | | SRPSARTVDLQMNNVKPEDTAVYYCAARLSGRTAG | |
| | | SRTYYAEGQYDYRGQGTQVTVSS | |
| 94 | NBX0818 | QVQLQQSGGGLVQAGGSLRLSCAASGRTSSSSYLG | PirA |
| | | WFRQAPGKEREFVASIRWSDGSTYYRDSVEGRFTI | |
| | | SRDNAKNTVYLRMNSLKPEDTAVYYCAAATTDWGP | |
| | | RGPYNYWGQGTLVTVSS | |
| 95 | NBX0819 | QVQLQQSGGREVRPGDSLRLSCRASGRTSGAWNMA | PirA |
| | | WFRQAPGKDREFVAAISGSGRTTEYADSAKGRFTI | |
| | | SRDMAKNTVYLQIVINSLKPEDTAVYNCAASTFDW | |
| | | GPRGPYRLWGQGTQVTVSS | |
| 96 | NBX0820 | QVQLQESGGGLVQTGGSLRLSCAASGRTFSNYVIG | PirA |
| | | WFRQAPGKEREFVAAVGRGINSAYHATHYSESVKD | |
| | | RFTTSRDNAKNTGFLQMNSLKTEDTAVYYCAVTSR | |
| | | WGQFDRTDFNSWGQGTQVTVSS | |
| 97 | NBX0821 | QVQLQESGGGLVQAGGSLRLSCVVSGMLFSIRNMR | PirA |
| | | WYRQAPGKQRELVAQIGTSGATDYVGSVEGRFTIS | |
| | | RDNPKNTVYLQMNSLKPEDTAVYFCNALNYWGEGT | |
| | | LVTVSS | |
| 98 | NBX0822 | QVQLQESGGGLVRPGDSLTLSCTYSGQTFTNSGMA | PirA |
| | | WFRQRPGKEREFVAAVSRSGLGRRYADSVRGRFTI | |
| | | TRDNGKNTANLQMDSLKPEDTAVYSCAATTLDWGP | |
| | | RGPYRYWGQGTQVTVSS | |
| 99 | NBX0823 | QVQLQQSGGGLVQTGGSLRLSCAASGSIFSIDFMG | PirA |
| | | WYRQAPGNPREFVARIRGGNTYYADSVKGRFNISR | |
| | | DNAENTVYMQMNSLKSEDTAVYYCNAQITMRGGTW | |
| | | STSEYWGQGTQVNVSS | |
| 100 | NBX0824 | QVQLQESGGGLVQAGGSLRLTCAASGRTLSSYLSS | PirA |
| | | YAMGWFRQAPGKERESVATITWNGDRTLYADAVKG | |
| | | RFTISRDNAKNTVYLQMNSVIPEDTAVYYCAADTV | |
| | | GRWRSTLSVRDEYDYWGQGTQVTVAS | |
| 101 | NBX0825 | QVQLQESGGGVVETGGSLSVSCVASGRTFSAYTMA | PirA |
| | | WFRQSPGKEREFVASMSRGSAAYYTDSVRGRFAIS | |
| | | RVGDKNTVHLQMRDLKPEDTAVYYCAGGSPGSSQI | |
| | | ATPEAYTYWGQGTQVTVSA | |
| 102 | NBX0826 | QVQLQESGGGLVQAGGSLRLSCAASGSISSSHVMG | PirB |
| | | WYRQAPGKQRELVATITSGGSTIYADSVKGRFTVS | |
| | | RDNAKNTVYLQMNSLKSEDTAVYYCHARRLWNTYW | |
| | | GQGTQVTVSS | |
| 103 | NBX0827 | QVQLQESGGGLVQAGGSLRLSCAASGSISSSFVMG | PirB |
| | | WYRQAPGKQRELVATITSGGSTIYADSVKGRFTVS | |
| | | RDNAKNTVYLQMNSLKSEDTAVYYCHARRLWNTYW | |
| | | GQGTQVTVSS | |
| 104 | NBX0828 | QVQLQESGGGAVQAGGSLRLSCAGPRSIFSGNAMA | PirB |
| | | WYRQVPGKQRETVATVNTGGLTWYGDFVKGRFTIS | |
| | | RDDAKNTLLLQMDSLKPEDTAVYYCNAVLVRARGM | |
| | | WGQGTQVTVSS | |
| 105 | NBX0829 | QVQLQESGGGSVQPGGSLRLSCSASGDRLSSYVMG | PirB |
| | | WYRQAPGKQRELVATVTSGGRTNYADSVKGRFTIS | |
| | | RDNAKNTVYLQMNSLKPEDTAVYYCNARILFTNYW | |
| | | GQGTQVTVSS | |
| 106 | NBX0830 | QVQLQESGGGLVQPGGSLQLSCVASGSVLSRYVMG | PirB |
| | | WYRQAPGKQRELVATITSGGITRYADSMKGRFTIS | |
| | | RDNAKNTVHLQMSSLKPEDTAVYYCNARALWNTYW | |
| | | GQGTQVTVSS | |
| 107 | NBX0831 | QVQLQESGGGLVQAGGSLRLSCSASGDAFSRYVMG | PirB |
| | | WYRQAPGKQRELVATITSGGSTIYADSVKGRFTVS | |
| | | RDNAKNTVYLQMNSLKSEDTAVYYCHARRLWNTYW | |
| | | GQGTQVTVSS | |
| 108 | NBX0832 | QVQLQQSGGGLVQAGGSLRLSCAASGSISSSYVMG | PirB |
| | | WYRQAPGKQRELVATITSGGSTIYADSVKGRFTVS | |
| | | RDNAKNTVYLQMNSLKSEDTAVYYCHARRLWNTYW | |
| | | GQGTQVTVSS | |
| 109 | NBX0833 | QVQLQQSGGGLVQAGGSLRLSCSASGSISSSHVMG | PirB |
| | | WYRQAPGKQRELVATITSGGSTIYADSVKGRFTVS | |
| | | RDNAKNTVYLQMNSLKSEDTAVYYCHARRLWDTYW | |
| | | GQGTQVTVSS | |
| 110 | NBX0834 | QVQLQESGGGSVQAGGSLRLSCAASESMFRDHNMG | PirB |
| | | WYRQAPGKQRELVATISRGGLINYGDSVRGRFTIS | |
| | | RDNAKNTIYLQMNSLKVEDTAVYYCNARRLLTTVW | |
| | | GQGTQVTVSP | |
| 111 | NBX0835 | QVQLQESGGGLVQPGGSLRLSCSASGNRFSSSYVM | PirB |
| | | GWYRQAPGKQRELVATVTSGGLTHFKDSVKGRFTI | |
| | | SRDNAKNTVYLQMNSLKPEDTAVYYCNARILLTNY | |
| | | WGQGTQVTISS | |
| 112 | NBX0836 | QVQLQQSGGGLVQPGGSLRLSCSASGIRLGSYVMG | PirB |
| | | YYRQAPGKQRELVATVTSGGTTNRADSVKGRFTIS | |
| | | RDNAKNAVYLQMNSLKPEDTAVYYCNARILFTNYW | |
| | | GQGTQVTVSS | |
| 113 | NBX0837 | QVQLQESGGGLVQPGGSLRLSCAASGIVEANHVMG | PirB |
| | | WYRQAPGKQRELVASITNGGLINSVDSVAGRFTIS | |
| | | RDNAKNTVYLQMNNLKPEDTAVYYCNARRLYQQYW | |
| | | GQGTQVTVSS | |
| 114 | NBX0838 | QVQLQESGGGLVQAGGSLRLSCRVSGRTVGSYAMG | PirB |
| | | WFRLQPGKERQFVAAIGWSGASTLYAESVKGRFTI | |
| | | SRDNAKNTVYLQMNSLKPEDTAVYYCAQRPSSRYA | |
| | | SRYLGDYAYWGQGTQVTVSS | |
| 115 | NBX0839 | QVQLQESGGGLVQAGGSLRLSCAASGSIGSDYVLG | PirB |
| | | WYRQAPGKQRELVATITSGGLTHYGDSVKGRFTIS | |
| | | RDNAKNTVYVQMNSLKFEDTAIYYCNARRLFRNYW | |
| | | GQGTQVTVSS | |
| 116 | NBX0840 | QVQLQQSGGGLVQAGGSLRLSCAASGSIRSSNVMG | PirB |
| | | WYRQTPGKQRELVATMTAGGLTNYADSVKGRFTIS | |
| | | RDNAKNTVYLQMNSLKPEDTAVYYCHYRRIFNVYW | |
| | | GQGTQVTVSS | |
| 117 | NBX0841 | QVQLQESGGGLVQAGGSLRLSCAASARTFIYKMGW | PirB |
| | | FRQAPGKERDFVASIMWSVGNNYYYTDSAKGRFTI | |
| | | SRDIAKNTMYLQMDSLEPEDTGEYYCAAATTSTQW | |
| | | RYWGQGTQVTVSS | |
| 118 | NBX0842 | QVQLQESGGGWVQPGGSLRLSCAASGSIDNGYVMG | PirB |
| | | WYRQAPGKQRELVATITSGTNTHYADSVKGRFTIS | |
| | | RDNAKTTVYLQMNSLKPEDTAVYYCLARRLFTMYW | |
| | | GQGTQVTVSS | |
| 119 | NBX0843 | QVQLQESGGGLVQPGGSLRLSCSASGNRFSSSYVM | PirB |
| | | GYYRQAPGKQRELVATVTTGGLTNYADSVKGRFTI | |
| | | SRDNAKNTVYLQMNSLKPEDTAVYYCNARILLTNY | |
| | | WGQGTQVTVSS | |
| 120 | NBX0844 | QVQLQESGGGLAQTGDSLRLSCAASGRMFSGFVMG | PirB |
| | | WYRQNPGKQRELVATITNGGLTHYGDSVKGRFTIS | |
| | | RDNAKNTVYLQMNSLKSEDSAVYYCNARRLFTNYW | |
| | | GQGTQVTVSP | |
| 121 | NBX0845 | QVQLQESGGGLVQAGGSLRLSCAASGRTFEATYMG | PirA |
| | | WFRQSPGKEREFVAAISWGGGTTYYGDSVKGRFTV | |
| | | SRDNAKNTAYLQMNSLKLEDTAVYSCAAATVDWGP | |
| | | RGPYRYWGQGTQVTVSS | |
| 122 | NBX0846 | QVQLQESGGGLVQAGGSLRLSCAASGRTFSTYYKG | PirA |
| | | WFRQAPGKEREFLAAISDGGTYYADSVKGRFTISR | |
| | | DNAKNTVYLQMNSLKPEDTAVYYCAAQGVWRGTGS | |
| | | YTWQYSYDYWGQGTQVTVSS | |
| 123 | NBX0849 | QVQLQESGGGLVQAGGSLRLSCAASGRTFNRYAMG | PirA |
| | | WFRQAPGKEREFVAAISWSGNTQYTDSVKGRFTIS | |
| | | RDDAKNTVYLQMNSLKPEDTAVYYCALRIPASSST | |
| | | TYYYADQYDYWGQGTQVTVSS | |
| 124 | NBX0850 | QVQLQESGGGLVQAGGSLRLSCAASGSISASYVMG | PirB |
| | | WYRQTPGKQRELVATTTSGGTTRYADSVRGRFTIS | |
| | | RDNARNTVYLQMNSLKPDDTAVYYCNARRLFLNYW | |
| | | GQGTQVTVSS | |
| 125 | NBX0851 | QVQLQQSGGGLVQPGGSLRLSCAASGRMFSGYVMG | PirB |
| | | WYRQAPGKQRELVATITNGGLTNYADSVKGRFTIS | |
| | | RDNAKNTVYLQMNSLKPDDTAVYYCNARRLWDIYW | |
| | | GQGTQVTVSS | |
| 126 | NBX0852 | QVQLQESGGGLVQAGGSLRLSCEASGRTFSSYRVG | PirB |
| | | WFRQAPGKGREFVAAISATGGTTYYGDSVKGRFTI | |
| | | SRDNAENTVSLQMNSLEPEDTAVYYCAATKGIVNY | |
| | | RVAGTYDAWGQGTQVTVSS | |
| 127 | NBX0853 | QVQLQESGGGLVQAGGSLRLSCSASGSISSSHVMG | PirB |
| | | WYRQAPGKQRELVATITNGGLTHYADSVKGRFTIS | |
| | | RDNAKNTVYLQMNSLKPDDTAVYYCNARRLWDNYW | |
| | | GQGTQVTVSS | |
| 128 | NBX0854 | QVQLQQSGGGLVQAGGSLRISCAASGSISSAYVMG | PirB |
| | | WYRQAPGTQRELVATITSGGTTNYADSVKGRFTVS | |
| | | RDNAKNTVYLQMNSLKPDDTAVYYCNARRLWTTYW | |
| | | GQGTQVTVSS | |
| 129 | NBX0855 | QVQLQESGGGLVQPGESLRLSCAASTSGFSSYVMA | PirB |
| | | WYRQAPGKQRELVASMTTGGLTNYADSVKGRFTIS | |
| | | RDNAKNTVYLQMNSLKPEDTAAYYCNARRLWNAYW | |
| | | GQGTQVTVSS | |
| 130 | NBX0856 | QVQLQESGGGLVQAGGSLRLSCVSSGSISASHVMG | PirB |
| | | WYRQAPGKQRELVATITSGGTTRYADSVKGRFTVS | |
| | | RDNAKNTVYLQMNDLKSEDTAVYYCHARRLWDTYW | |
| | | GQGTQVTVSS | |
| 131 | NBX0857 | QVQLQQSGGGLVQPGGSLRLSCAASGRIFSGHVMG | PirB |
| | | WYRQAPGKQRELVATITNGGLTNYGDSVKGRFTIS | |
| | | RDNAKNTVYLQMNSLKPDDTAVYYCNARRLWDTYW | |
| | | GQGTQVTVSS | |
| 132 | NBX0858 | QVQLQESGGGLVQAGGSLRLSCAASRRDFTTTTMA | PirB |
| | | WYRQAPGKKRETVATVNTGGLTWYADFVKGRFTIS | |
| | | RDDAVNTLLLQMDSLKPEDTAVYYCNAVLVRARGM | |
| | | WGQGTQVTVSS | |
| 133 | NBX0859 | QVQLQESGGGLVQPGGSLRLSCSASGNRFSSSYVM | PirB |
| | | GWYRQGPGKQRELVATVTSGGMTHYGDSVKGRFTI | |
| | | SRDNAKNTVYLHMNSLKPEDTGVYYCFARRLWDIH | |
| | | WGQGTQVTVSS | |
| 134 | NBX0860 | QVQLQESGGGLAQAGGSLGLSCAASETEDSSHVMG | PirB |
| | | WYRQAPGKQRELVATITSGGLTNYADSAKGRFTIS | |
| | | RDNAKNAVYLQMNSLKPEDTAVYYCHARRLFRVYW | |
| | | GQGTQVTVSS | |
| 135 | NBX0861 | QVQLQESGGGLVQAGGSLRLSCVASGSISNSHVMG | PirB |
| | | WYRQAPGKERELVATLTSGGLTHFGDSVKGRFTIS | |
| | | RDNAKNTIYLQMNSLKVEDTAVYYCNARRLLTSMW | |
| | | GQGTQVTVSP | |
| 136 | NBX0862 | QVQLQESGGGLVQPGGSLRLSCSASGNRFSSSYVL | PirB |
| | | GWYRQAPGKQRELVATVTSGGLTHYGDSVKGRFTI | |
| | | SRDNAKNTVYLQMNSLKPEDTAVYYCNARILLTNY | |
| | | WGQGTQVTVSS | |
| 137 | NBX0863 | QVQLQQSGGGLAQAGGSLRLSCAASGRTFNWYTMA | PirB |
| | | WFRQAPGKEREFVAAIRLGSTVYGDSVKARFTISR | |
| | | DNAKSTVSLQMNSLKPEDTALYYCAVGITGDGTIQ | |
| | | GGPYQYWGQGTQVTVSS | |
| 138 | NBX0864 | QVQLQESGGGLVQPGGSLRLSCAASGIISSAYYMG | PirB |
| | | WYRQAPGKQRELVATUSGGTTRYADSVKGRITISR | |
| | | DNAKNTVLLQMNSLKPEDTAVYYCNARILLTNYWG | |
| | | QGTQVTVSS | |
| 139 | NBX0865 | QVQLQESGGGLVQAGGSLRLSCADSGRVFSTYVMG | PirB |
| | | WYRQVPGKQRELVATITPGGLINYGDAVKGRFTIS | |
| | | RDNAKNTVYLQMNSLKPADTAVYYCNARRLFAINW | |
| | | GGGTQVTVSS | |
| 140 | NBX09001 | QVQLQESGGGSVQPGGSLRLSCAASGSALSSNVLG | PirB |
| | | WYRQAPGKQRELVATISSGGLTNYADSVKGRFTIS | |
| | | RDNAKNTVYLQMNSLKPEDTAVYYCQSRRLFTVYW | |
| | | GQGTQVTVSS | |
| 141 | NBX09002 | QVQLQESGGGLVQAGESLRLSCADSGRTSSTYDMA | PirB |
| | | WFRQAPGKEREFVAAISRDGGRLSYADSVKGRFTI | |
| | | SRDNAKNTLSLQMNSLRPEDTAVYYCAAFSIRGGL | |
| | | RPSYKYWGQGTQVTVSS | |
| 142 | NBX09003 | QVQLQESGGGLVQPGGSLRLSCTASGSIFSLLNMG | PirB |
| | | WYRQAPGKQRELVASITSRSYTNYADSVKGRFTIS | |
| | | RDNTKNMVYLQMNSLKPEDTAVYYCNLNPADWGRL | |
| | | RNWGQGTQVTVSS | |
| 143 | NBX09004 | QVQLQESGGGVVQSGGSLRLSCAGPRSIFSGNAMA | PirB |
| | | WYRQAPGKQRETVATVNTGGLTWYVDFVKGRFTIS | |
| | | RDDAKNTLLLQMDSLKPEDTAVYYCNAVLVRARGM | |
| | | WGQGTQVTVSS | |
| 144 | NBX09005 | QVQLQESGGGLVQAGGSLRLSCAASGLTFGSYAMG | PirB |
| | | WFRQAPGKEREFVAAIMRYSSRTYYTDSVKGRFTI | |
| | | SRDNAKNTVNLQMNNLEPEDTAIYYCAAAKRLSIV | |
| | | TLPRQYEFWGQGTQVTVSS | |
| 145 | NBX09006 | QVQLQESGGGLVQPGGSLRLSCAASGSISGSYVMG | PirB |
| | | WYRQAPGKQRELVATITSGGLTRYADSVKGRWTIS | |
| | | RDNAKNTVYLQMNNLKLEDTAVYYCSARRIATTYW | |
| | | GQGTQVTVSS | |
| 146 | NBX09007 | QVQLQESGGGLVQPGGSLRLSCAASGSISSSYVMG | PirB |
| | | WYRQAPGKQRDLVATITNAGNIHYGDSVKGRFTIS | |
| | | RDNAKNTVSLQMNSLKPEDTAVYYCNARALWRAYW | |
| | | GQGTQVTVSS | |
| 147 | NBX09008 | QVQLQESGGGLVQAGGSLRLSCSASGRTFSVRAMG | PirB |
| | | WFRQAPGKERESVAAIHQNTRTTLYADSVKGRFAI | |
| | | SRDGTKNTVYLQMNSLKPEDTAVYYCAASDDYGLQ | |
| | | IKEVAYKYWGQGTQVTVAS | |
| 148 | NBX09009 | QVQLQESGGGLVQAGGSLRLSCAASGLTFGSYAMG | PirB |
| | | WFRQAPGKEREFVATIMRYSSRTYYTDSVKGRFTI | |
| | | SRDNAKNTVNLQMNNLEPEDAAIYYCAAAKRLSIV | |
| | | ALPRQYEFWGQGTQVTVSS | |
| 149 | NBX09010 | QVQLQQSGGGLVQAGGSLRLSCAASGLTFGSYAMG | PirB |
| | | WFRQAPGKEREFVAAIMRYSSRTYYTDSVKGRFTI | |
| | | SRDNAKNTVNLQMNNLEPEDTAIYYCAAAKRLSRV | |
| | | TLPREYEFWGQGTQVTVSS | |
| 150 | NBX09011 | QVQLQESGGGLVQPGESLRLSCAASTSGFSSYVMA | PirB |
| | | WYRQAPGKQRELVASMTTGGLTNYADSVKGRFTIS | |
| | | RDNAKNTVYLQMNSLKPEDTAAYYCNARRLWNAYW | |
| | | GQGTQVTVSS | |

TABLE 2
Unique SEQ IDs for V_{H}H CDRs of this disclosure

| | CDR1 Amino | CDR1 | CDR2 Amino | CDR2 | CDR3 Amino | CDR3 | |
|---|---|---|---|---|---|---|---|
| | Acid | SEQ | Acid | SEQ | Acid | SEQ | |
| NBX | Sequence | ID NO: | Sequence | ID NO: | Sequence | ID NO: | Antigen |
| NBX0401 | GRTFSSYTM | 7 | IDWWSSSS | 13 | AASGKYGLAYSRRDYAY | 19 | PirA |
| NBX0402 | GLPFINYAM | 8 | IDWNGDST | 14 | ASHYQPYIRVSATRRFEADY | 20 | PirA |
| NBX0403 | GLPFINYAM | 9 | IDWNGDST | 15 | AADYQPYIRVSATRRFEADY | 21 | PirA |
| NBX0404 | GRTFSRYTM | 10 | ISWSGT | 16 | ASGSRRLYYSSDIDY | 22 | PirB |
| NBX0405 | GRTFSRYTM | 11 | ISWSGT | 17 | VVGSRRLYYSSDINY | 23 | PirB |
| NBX0406 | GFTFSTYTW | 12 | ISRDGIT | 18 | AVVKEDNRYWCHADRNLYRN | 24 | VP24 |
| NBX0601 | RFTFSTYPM | 151 | ISVGGGIK | 273 | AKGGKTSYTRE | 395 | PirA |
| NBX0602 | GRTFSRYAM | 152 | VDWSGGST | 274 | AARARDVYGRAWYVEDSSTYDY | 396 | PirA |
| NBX0603 | GSMFSINAM | 153 | ISRGGIT | 275 | NAENRRLGDDF | 397 | PirA |
| NBX0604 | GRTFSSYTM | 154 | IRWSGGSR | 276 | AADRDGYSSYAHQYDY | 398 | PirA |
| NBX0605 | GSFFSIYAM | 155 | ITSGSET | 277 | NRWAPLTRLDY | 399 | PirA |
| NBX0606 | GRTSSSFAM | 156 | ITRTGGRT | 278 | AARWATATSNSIRVYYNEGQYDY | 400 | PirA |
| NBX0607 | GGTFSRLTM | 157 | VSWVAETT | 279 | AAGPRDMIRSRNIRSYDS | 401 | PirA |
| NBX0608 | GSMFNINAM | 158 | ISSGGIT | 280 | NAENRRLGDDY | 402 | PirA |
| NBX0609 | GSIFSGNVM | 159 | ISSGGIT | 281 | NLENRRLGDDY | 403 | PirA |
| NBX0610 | GSIFSRDAM | 160 | ISSGGIT | 282 | NAENRRLGDDY | 404 | PirA |
| NBX0611 | GGTFSRLTM | 161 | VSWVAETT | 283 | AAGPRDMIRSRNIKSYDS | 405 | PirA |
| NBX0612 | GTIFSINKM | 162 | ARSGGII | 284 | NALIHTRYDRVTGY | 406 | PirA |
| NBX0613 | GSIFSINAM | 163 | ISSGGIT | 285 | NAENPRLGDDYW | 407 | PirA |
| NBX0614 | GSSFRSNAI | 164 | ITRSGST | 286 | HDETMKLISVKNDY | 408 | PirB |
| NBX0615 | GSMFSRNAM | 165 | ISSGGIT | 287 | NAENRRLGDDY | 409 | PirA |
| NBX0616 | GLTFSSYAM | 166 | ISWSGKST | 288 | AADYQRLGLLRVGVAEYDY | 410 | PirA |
| NBX0617 | GRSGSTSFM | 167 | IRWSSGMT | 289 | AADNYPLHIGHQDHEVDY | 411 | PirA |
| NBX0618 | GSIFSFNAM | 168 | ITKGGST | 290 | NVKTLRSALFPGYEY | 412 | PirA |
| NBX0619 | GGTFSRLTM | 169 | VSWVAETT | 291 | AAGPRDMIRSRNIRSYDS | 413 | PirA |
| NBX0620 | GGTFSRLTL | 170 | VSWVAEMT | 292 | AAGPRDMVRSRNIRSYDS | 414 | PirA |
| NBX0621 | GRTFSTYSM | 171 | IRWSGGSR | 293 | AADRDGYSSYAHQYDY | 415 | PirA |
| NBX0622 | GRLFNINAM | 172 | ISSGGIT | 294 | NAENRGLGDDY | 416 | PirA |
| NBX0623 | GRTLSNYAM | 173 | ISRSGMST | 295 | AARGGLPNPSRTYGFEEQYDY | 417 | PirA |
| NBX0624 | GRTVSSLPM | 174 | LNWSGTST | 296 | AAKGAIYYSYSPRNNNNYDV | 418 | PirA |
| NBX0625 | GSMFNINAM | 175 | ISSGGIT | 297 | NAENRLGDDY | 419 | PirA |
| NBX0626 | ETTLATNAM | 176 | ISSVSSGGIT | 298 | NGVRRGRSY | 420 | PirB |
| NBX0627 | GSSFRSNAI | 177 | ITRSGST | 299 | HDETMKLITGKNDY | 421 | PirB |
| NBX0628 | GRTFSSYAM | 178 | IKWSGGSR | 300 | AADRDGYSRYAHQYDY | 422 | PirA |
| NBX0629 | GGTFSRLTI | 179 | VSWVAETT | 301 | AAGPRDMIRSRNIRSYDS | 423 | PirA |
| NBX0630 | GVTFSDYPM | 180 | ISAGGGLI | 302 | NLKTSYFWPW | 424 | PirA |
| NBX0631 | GGTFSRLTI | 181 | VSWVAQTT | 303 | AAGPQDMIRSRNIRSYIS | 425 | PirA |
| NBX0632 | GGTFSRLTM | 182 | VSWVAGTT | 304 | AAGPRDMIRSRNIRSYDS | 426 | PirA |
| NBX0633 | GTIFRSKSM | 183 | ISGLGHT | 305 | NTFTAAFS | 427 | PirA |
| NBX0634 | GGSFSRLTL | 184 | VSWVAETT | 306 | AAGPRDMIRSRNIRSYAS | 428 | PirA |
| NBX0635 | GLTFSNYAM | 185 | ISWSGKST | 307 | AAEYQRLGLLRDGVADYSY | 429 | PirA |
| NBX0636 | GGTFSRLTM | 186 | VSWVAETT | 308 | AAGPRDMIRSRNIRSYVS | 430 | PirA |
| NBX0637 | GRTFSSYPM | 187 | ISAGGGLI | 309 | NLKTSYFWP | 431 | PirA |
| NBX0638 | GSIFSGNVM | 188 | ITGGGIT | 310 | NYRRIMQQY | 432 | PirB |
| NBX0639 | GIVFSSIVM | 189 | ITNGGLV | 311 | NARRIMTSY | 433 | PirB |
| NBX0640 | GSIFSGNVM | 190 | ITGGGIT | 312 | HYRRIMQQY | 434 | PirB |
| NBX0641 | GSISNSYVM | 191 | ITSGGLT | 313 | NARVIFTTY | 435 | PirB |
| NBX0642 | TVTFSRYAM | 192 | ISGSGHRT | 314 | AGDLVAKFDSAYRVSYDS | 436 | PirB |
| NBX0643 | GSIFSGNVM | 193 | MTGGGVT | 315 | HYRRIMQQY | 437 | PirB |
| NBX0644 | GSIFSGNVM | 194 | ITGGGIT | 316 | FYRRIMQQY | 438 | PirB |
| NBX0645 | GSIFSGNVM | 195 | ITGGGIT | 317 | LYRRIMQQY | 439 | PirB |
| NBX0646 | GSIFSGNVM | 196 | ITGGGVT | 318 | HYRRIMQQS | 440 | PirB |
| NBX0647 | GSIFSGNVM | 197 | MTGGGVT | 319 | HYRRIMQQY | 441 | PirB |
| NBX0648 | GIIFSSNVM | 198 | RTSGGLT | 320 | NARRLFTNY | 442 | PirB |
| NBX0649 | GSIASGNVL | 199 | ITSGGLT | 321 | NYRRLATGY | 443 | PirB |
| NBX0650 | GSIFSGNVL | 200 | ITGGGIT | 322 | HYRRIMQQY | 444 | PirB |
| NBX0722 | GRTFSSYPM | 201 | ISRSGDPG | 323 | AARQIYSNNYSY | 445 | VP28 |
| NBX0723 | GRTFSSYPM | 202 | ISRSGVPG | 324 | AARSIYSNNYSY | 446 | VP28 |
| NBX0724 | GRTFSSYPM | 203 | ISRSGVSG | 325 | AARSIYSNNYSY | 447 | VP28 |
| NBX0725 | GRTFSSYPM | 204 | ISRSGNPG | 326 | AARQIYSNNYSY | 448 | VP28 |
| NBX0730 | GRTFSSYPM | 205 | INRNGNIP | 327 | AARTIYDSHYTS | 449 | VP28 |
| NBX0737 | DGAITGSYYVW | 206 | ITYDGST | 328 | ARAGEEYICSGYGCHGSLGLDY | 450 | VP24 |
| NBX0738 | GFTFGSYAM | 207 | INSGDGRI | 329 | ATGIRTPII | 451 | VP24 |
| NBX0739 | GSIFTSDV | 208 | MTSAGTT | 330 | GVGRF | 452 | VP24 |
| NBX0745 | GRTFSSYPM | 209 | ISRSGDPG | 331 | AAREIYSNNYSY | 453 | VP28 |
| NBX0746 | GRTFSSYAM | 210 | ISRSGNPG | 332 | AARQIYSNNYSY | 454 | VP28 |
| NBX0813 | GMLFSIRNM | 211 | IGSSGNT | 333 | NALNY | 455 | PirA |
| NBX0814 | MRTFNSRTI | 212 | IAWTGGN | 334 | AAQTRPYDLPSERPDDYAS | 456 | PirA |
| NBX0815 | GRTFSSYAM | 213 | VSWSGGST | 335 | AAQRVMDYYRPRTESAYAY | 457 | PirA |
| NBX0816 | EYIFSNFGM | 214 | ISRSGSRM | 336 | AAVYGQYSYHYSSDSKQYSY | 458 | PirA |
| NBX0817 | GGTNSNYAM | 215 | LSWSGYNT | 337 | AARLSGRTAGSRTYYAEGQYDY | 459 | PirA |
| NBX0818 | GRTSSSSYL | 216 | IRWSDGST | 338 | AAATTDWGPRGPYNY | 460 | PirA |
| NBX0819 | GRTSGAWNM | 217 | ISGSGRTT | 339 | AASTFDWGPRGPYRL | 461 | PirA |
| NBX0820 | GRTFSNYVI | 218 | VGRGINSAYHAT | 340 | AVTSRWGQFDRTDFNSW | 462 | PirA |
| NBX0821 | GMLFSIRNM | 219 | IGTSGAT | 341 | NALNY | 463 | PirA |
| NBX0822 | GQTFTNSGM | 220 | VSRSGLGR | 342 | AATTLDWGPRGPYRY | 464 | PirA |
| NBX0823 | GSIFSIDFM | 221 | IRGGNT | 343 | NAQITMRGGTWSTSEY | 465 | PirA |
| NBX0824 | GRTLSSYLSSYAM | 222 | ITWNGDRT | 344 | AADTVGRWRSTLSVRDEYDY | 466 | PirA |
| NBX0825 | GRTFSAYTM | 223 | MSRGSAA | 345 | AGGSPGSSQIATPEAYTY | 467 | PirA |
| NBX0826 | GSISSSHVM | 224 | ITSGGST | 346 | HARRLWNTY | 468 | PirB |
| NBX0827 | GSISSSFVM | 225 | ITSGGST | 347 | HARRLWNTY | 469 | PirB |
| NBX0828 | RSIFSGNAM | 226 | VNTGGLT | 348 | NAVLVRARGM | 470 | PirB |
| NBX0829 | GDRLSSYVM | 227 | VTSGGRT | 349 | NARILFTNY | 471 | PirB |
| NBX0830 | GSVLSRYVM | 228 | ITSGGIT | 350 | NARALWNTY | 472 | PirB |
| NBX0831 | GDAFSRYVM | 229 | ITSGGST | 351 | HARRLWNTY | 473 | PirB |
| NBX0832 | GSISSSYVM | 230 | ITSGGST | 352 | HARRLWNTY | 474 | PirB |
| NBX0833 | GSISSSHVM | 231 | ITSGGST | 353 | HARRLWDTY | 475 | PirB |
| NBX0834 | ESMFRDHNM | 232 | ISRGGLI | 354 | NARRLLTTV | 476 | PirB |
| NBX0835 | GNRFSSSYVM | 233 | VTSGGLT | 355 | NARILLTNY | 477 | PirB |
| NBX0836 | GIRLGSYVM | 234 | VTSGGTT | 356 | NARILFTNY | 478 | PirB |
| NBX0837 | GIVFANHVM | 235 | ITNGGLI | 357 | NARRLYQQY | 479 | PirB |
| NBX0838 | GRTVGSYAM | 236 | IGWSGAST | 358 | AQRPSSRYASRYLGDYAY | 480 | PirB |
| NBX0839 | GSIGSDYVL | 237 | ITSGGLT | 359 | NARRLFRNY | 481 | PirB |
| NBX0840 | GSIRSSNVM | 238 | MTAGGLT | 360 | HYRRIFNVY | 482 | PirB |
| NBX0841 | ARTFIYKM | 239 | IMWSVGNNY | 361 | AAATTSTQWRY | 483 | PirB |
| NBX0842 | GSIDNGYVM | 240 | ITSGTNT | 362 | LARRLFTMY | 484 | PirB |
| NBX0843 | GNRFSSSYVM | 241 | VTTGGLT | 363 | NARILLTNY | 485 | PirB |
| NBX0844 | GRMFSGFVM | 242 | ITNGGLT | 364 | NARRLFTNY | 486 | PirB |
| NBX0845 | GRTFEATYM | 243 | ISWGGGIT | 365 | AAATVDWGPRGPYRY | 487 | PirA |
| NBX0846 | GRTFSTYYK | 244 | ISDGGT | 366 | AAQGVWRGTGSYTWQYSYDY | 488 | PirA |
| NBX0849 | GRTFNRYAM | 245 | ISWSGNT | 367 | ALRIPASSSTTYYYADQYDY | 489 | PirA |
| NBX0850 | GSISASYVM | 246 | TTSGGTT | 368 | NARRLFLNY | 490 | PirB |
| NBX0851 | GRMFSGYVM | 247 | ITNGGLT | 369 | NARRLWDIY | 491 | PirB |
| NBX0852 | GRTFSSYRV | 248 | ISATGGTT | 370 | AATKGIVNYRVAGTYDA | 492 | PirB |
| NBX0853 | GSISSSHVM | 249 | ITNGGLTH | 371 | NARRLWDNY | 493 | PirB |
| NBX0854 | GSISSAYVM | 250 | ITSGGTT | 372 | NARRLWTTY | 494 | PirB |
| NBX0855 | TSGFSSYVM | 251 | MTTGGLT | 373 | NARRLWNAY | 495 | PirB |
| NBX0856 | GSISASHVM | 252 | ITSGGTT | 374 | HARRLWDTY | 496 | PirB |
| NBX0857 | GRIFSGHVM | 253 | ITNGGLT | 375 | NARRLWDTYW | 497 | PirB |
| NBX0858 | RRDFTTTTM | 254 | VNTGGLT | 376 | NAVLVRARGM | 498 | PirB |
| NBX0859 | GNRFSSSYVM | 255 | VTSGGMT | 377 | FARRLWDIH | 499 | PirB |
| NBX0860 | ETIDSSHVM | 256 | ITSGGLT | 378 | HARRLFRVY | 500 | PirB |
| NBX0861 | GSISNSHVM | 257 | LTSGGLT | 379 | NARRLLTSM | 501 | PirB |
| NBX0862 | GNRFSSSYVL | 258 | VTSGGLT | 380 | NARILLTNY | 502 | PirB |
| NBX0863 | GRTFNWYTM | 259 | IRLGST | 381 | AVGITGDGTIQGGPYQY | 503 | PirB |
| NBX0864 | GIISSAYVM | 260 | ITSGGTT | 382 | NARILLTNY | 504 | PirB |
| NBX0865 | GRVFSTYVM | 261 | ITPGGLI | 383 | NARRLFAIN | 505 | PirB |
| NBX09001 | GSALSSNVL | 262 | ISSGGLT | 384 | QSRRLFTVY | 506 | PirB |
| NBX09002 | GRTSSTYDM | 263 | ISRDGGRL | 385 | AAFSIRGGLRPSYKY | 507 | PirB |
| NBX09003 | GSIFSLLNM | 264 | ITSRSYT | 386 | NLNPADWGRLRN | 508 | PirB |
| NBX09004 | RSIFSGNAM | 265 | VNTGGLT | 387 | NAVLVRARGM | 509 | PirB |
| NBX09005 | GLTFGSYAM | 266 | IMRYSSRT | 388 | AAAKRLSIVTLPRQYEF | 510 | PirB |
| NBX09006 | GSISGSYVM | 267 | ITSGGLT | 389 | SARRIATTY | 511 | PirB |
| NBX09007 | GSISSSYVM | 268 | ITNAGNI | 390 | NARALWRAY | 512 | PirB |
| NBX09008 | GRTFSVRAM | 269 | IHQNTRTT | 391 | AASDDYGLQIKEVAYKY | 513 | PirB |
| NBX09009 | GLTFGSYAM | 270 | IMRYSSRT | 392 | AAAKRLSIVALPRQYEF | 514 | PirB |
| NBX09010 | GLTFGSYAM | 271 | IMRYSSRT | 393 | AAAKRLSRVTLPREYEF | 515 | PirB |
| NBX09011 | TSGFSSYVM | 272 | MTTGGLT | 394 | NARRLWNAY | 516 | PirB |

### Antibodies Recombinantly Expressed

In another aspect, the present invention provides a method for producing V_{H}H in a suitable producing organism. Suitable producing organisms include, without limitation, bacteria, yeast and algae. In certain embodiments, the producing bacterium is Escherichia coli. In certain embodiments, the producing bacterium is a member of the Bacillus genus. In certain embodiments, the producing bacterium is a probiotic. In certain embodiments, the yeast is Pichia pastoris. In certain embodiments, the yeast is Saccharomyces cerevisiae. In certain embodiments, the algae is a member of the Chlamydomonas or Phaeodactylum genera.

### Antibodies Added to Feed

In yet another aspect, the present invention provides a polypeptide or pluralities thereof comprising a V_{H}H or V_{H}Hs that bind disease-causing agents and are administered to host animals via any suitable route as part of a feed product. In certain embodiments, the animal is selected from the list of host animals described, with that list being representative but not limiting. In certain embodiments, the route of administration to a recipient animal can be, but is not limited to: introduction to the alimentary canal orally or rectally, provided to the exterior surface (for example, as a spray or submersion), provided to the medium in which the animal dwells (including air and water based media), provided by injection, provided intravenously, provided via the respiratory system, provided via diffusion, provided via absorption by the endothelium or epithelium, or provided via a secondary organism such as a yeast, bacterium, algae, bacteriophages, plants and insects. In certain embodiments, the host animal is a shellfish. In certain embodiments, the host animal is shrimp.

### Feed Product

In a further aspect, the present invention provides a polypeptide or pluralities thereof comprising a V_{H}H or V_{H}Hs that bind disease-causing agents and are administered to host animals in the form of a product. The form of the product is not limited, so long as it retains binding to the disease-causing agent in the desired form. In certain embodiments, the product is feed, pellet, nutritional supplement, premix, therapeutic, medicine, or feed additive, but is not limited to these forms.

### Feeding Dosage

In a further aspect, the present invention provides a polypeptide or pluralities thereof comprising a V_{H}H or V_{H}Hs that bind disease-causing agents and are administered to host animals as part of a product at any suitable dosage regime. In practice, the suitable dosage is the dosage at which the product offers any degree of protection against a disease-causing agent, and depends on the delivery method, delivery schedule, the environment of the recipient animal, the size of the recipient animal, the age of the recipient animal and the health condition of the recipient animal among other factors. In certain embodiments, V_{H}Hs are administered to recipient animals at a concentration in excess of 1 mg/kg of body weight. In certain embodiments, V_{H}Hs are administered to recipient animals at a concentration in excess of 5 mg/kg of body weight. In certain embodiments, V_{H}Hs are administered to recipient animals at a concentration in excess of 10 mg/kg of body weight. In certain embodiments, V_{H}Hs are administered to recipient animals at a concentration in excess of 50 mg/kg of body weight. In certain embodiments, V_{H}Hs are administered to recipient animals at a concentration in excess of 100 mg/kg of body weight. In certain embodiments, V_{H}Hs are administered to recipient animals at a concentration less than 1 mg/kg of body weight. In certain embodiments, V_{H}Hs are administered to recipient animals at a concentration less than 500 mg/kg of body weight. In certain embodiments, V_{H}Hs are administered to recipient animals at a concentration less than 100 mg/kg of body weight. In certain embodiments, V_{H}Hs are administered to recipient animal at a concentration less than 50 mg/kg of body weight. In certain embodiments, V_{H}Hs are administered to recipient animals at a concentration less than 10 mg/kg of body weight.

### Feeding Frequency

In a further aspect, the present invention provides a polypeptide or pluralities thereof comprising a V_{H}H or V_{H}Hs that bind disease-causing agents and are administered to host animals as part of a product at any suitable dosage frequency. In practice, the suitable dosage frequency is that at which the product offers any protection against a disease-causing agent, and depends on the delivery method, delivery schedule, the environment of the recipient animal, the size of the recipient animal, the age of the recipient animal and the health condition of the recipient animal, among other factors. In certain embodiments, the dosage frequency can be but is not limited to: constantly, at consistent specified frequencies under an hour, hourly, at specified frequencies throughout a 24-hour cycle, daily, at specified frequencies throughout a week, weekly, at specified frequencies throughout a month, monthly, at specified frequencies throughout a year, annually, and at any other specified frequency greater than 1 year.

### Feed Additives

In a further aspect, the present invention provides a polypeptide or pluralities thereof comprising a V_{H}H or V_{H}Hs that bind disease-causing agents and are administered to host animals as part of a product that also comprises other additives or coatings. In practice, the most suitable coating or additive depends on the method of delivery, the recipient animal, the environment of the recipient, the dietary requirements of the recipient animal, the frequency of delivery, the age of the recipient animal, the size of the recipient animal, the health condition of the recipient animal In certain embodiments, these additives and coatings can include, but are not limited to the following list and mixtures thereof: a vitamin, an antibiotic, a hormone, 1 peptide, a steroid, a probiotic, a bacteriophage, chitin, chitosan, B-1,3-glucan, vegetable extracts, peptone, shrimp meal, krill, algae, B-cyclodextran, alginate, gum, tragacanth, pectin and gelatin.

### Non-Feed Uses

In a further aspect, the present invention provides a polypeptide or pluralities thereof comprising a V_{H}H or V_{H}Hs that bind disease-causing agents, and can be used in a non-feed use, such as but not limited to: a diagnostic kit, an ELISA-based assay, a western blot assay, an immunofluorescence assay, or a FRET assay, in its current form and/or as a polypeptide conjugated to another molecule. In certain embodiments, the conjugated molecule is can be but is not limited to: a fluorophore, a chemiluminescent substrate, an antimicrobial peptide, a nucleic acid or a lipid.

### Antigens

In a further aspect, the present invention provides a polypeptide or pluralities thereof comprising a V_{H}H or V_{H}Hs that bind disease-causing agents, including toxins, produced by a species of Vibrio. In certain embodiments, the Vibrio species is capable of harbouring the pVA-1 plasmid. In certain embodiments, the species does not belong to the Vibrio genus but is capable of harbouring disease-causing agents shared by Vibrio species, such as but not limited to the pVA-1 plasmid. In certain embodiments, the Vibrio species refers to both current and reclassified organisms. In certain embodiments, the Vibrio species is V. adaptatus, V. aerogenes, V. aestivus, V. aestuarianus, V. agarivorans, V. albensis, V. alfacsensis, V. alginolyticus, V. anguillarum, V. areninigrae, V. artabrorum, V. atlanticus, V. atypicus, V. azureus, V. brasiliensis, V. bubulus, V. calviensis, V. campbellii, V. casei, V. chagasii, V. cholerae, V. cincinnatiensis, V. coralliilyticus, V. crassostreae, V. cyclitrophicus, V. diabolicus, V. diazotrophicus, V. ezurae, V. fluvialis, V. fortis, V. furnissii, V. gallicus, V. gazogenes, V. gigantis, V. halioticoli, V. harveyi, V. hepatarius, V. hippocampi, V. hispanicus, V. ichthyoenteri, V. indicus, V. kanaloae, V. lentus, V. litoralis, V. logei, V. mediterranei, V. metschnikovii, V. mimicus, V. mytili, V. natriegens, V. navarrensis, V. neonatus, V. neptunius, V. nereis, V. nignpulchritudo, V. ordalii, V. orientalis, V. pacinii, V. parahaemolyticus, V. pectenicida, V. penaeicida, V. pomeroyi, V. ponticus, V. proteolyticus, V. rotiferianus, V. ruber, V. rumoiensis, V. salmonicida, V. scophthalmi, V. splendidus, V. superstes, V. tapetis, V. tasmaniensis, V. tubiashii, V. vulnificus, V. wodanis, V. xuii, V. fischer, or V. hollisae.

In certain embodiments, the V_{H}H or plurality thereof is capable of binding to two or more disease-causing agents, originating from the same or different species. In certain embodiments, the disease-causing agent is a polypeptide with 60%, 70%, 80%, 90%, 95%, 98%, 99%, or 100% amino acid sequence identity to PirA (SEQ ID 25). In certain embodiments, the disease-causing agent is a polypeptide with 60%, 70%, 80%, 90%, 95%, 98%, 99%, or 100% amino acid sequence identity to PirB (SEQ ID 26). In certain embodiments, the disease-causing agent is an exposed peptide, protein, protein complex, nucleic acid, lipid, or combination thereof, that is associated to the surface of the Vibrio bacterium. In certain embodiments, the disease-causing agent is a pilus, fimbria, flagellum, secretion system or porin. In certain embodiments, the disease-causing agent is the Vibrio bacterium.

In a further aspect, the present invention provides a polypeptide or pluralities thereof comprising a V_{H}H or V_{H}Hs that bind disease-causing agents produced by White Spot Syndrome Virus. In certain embodiments, the disease-causing agent is a polypeptide with 60%, 70% 80%, 90%, 95%, 98%, 99%, or 100% amino acid sequence identity VP24 (SEQ ID 27). In certain embodiments, the disease-causing agent is a polypeptide with 60%, 70% 80%, 90%, 95%, 98%, 99%, or 100% amino acid sequence identity to VP28 (SEQ ID 28). In certain embodiments, the disease-causing agent is viral protein associated with or hypothesised to be associated with the envelope of the White Spot Syndrome Virus. In certain embodiments, the disease-causing agent is the White Spot Syndrome Virus.

SEQ ID 25:
PirA
>tr|A0A085YLC0|A0A085YLC0_VIBPH JHE-like toxin
PirA-like OS = Vibrio parahaemolyticus OX = 670
GN = vp19 PE = 4 SV = 1
MSNNIKHETDYSHDWTVEPNGGVTEVDSKHTPIIPEVGRSVDIENTGRG
ELTIQYQWGAPFMAGGWKVAKSHVVQRDETYHLQRPDNAFYHQRIVVIN
NGASRGFCTIYYH
SEQ ID 26:
PirB
>tr|A0A085YLC1|A0A085YLC1_VIBPH JHE-like toxin
PirB-like OS = Vibrio parahaemolyticus OX = 670
GN = BTO19_25780 PE = 4 SV = 1
MTNEYVVTMSSLTEFNPNNARKSYLFDNYEVDPNYAFKAMVSFGLSNIP
YAGGFLSTLWNIFWPNTPNEPDIENIWEQLRDRIQDLVDESIIDAINGI
LDSKIKETRDKIQDINETIENFGYAAAKDDYIGLVTHYLIGLEENFKRE
LDGDEWLGYAILPLLATTVSLQITYMACGLDYKDEFGFTDSDVHKLTRN
IDKLYDDVSSYITELAAWADNDSYNNANQDNVYDEVMGARSWCTVHGFE
HMLIWQKIKELKKVDVFVHSNLISYSPAVGFPSGNFNYIATGTEDEIPQ
PLKPNMFGERRNRIVKIESWNSIEIHYYNRVGRLKLTYENGEVVELGKA
HKYDEHYQSIELNGAYIKYVDVIANGPEAIDRIVFHFSDDRTFVVGENS
GKPSVRLQLEGHFICGMLADQEGSDKVAAFSVAYELFHPDEFGTEK
SEQ ID 27:
VP24
>tr|Q9E7K6|Q9E7K6_WSSV Major structural protein
VP24 OS = White spot syndrome virus OX = 92652
GN = VP24 PE = 4 SV = 1
MHMWGVYAAILAGLTLILVVISIVVTNIELNKKLDKKDKDAYPVESEII
NLTINGVARGNHFNFVNGTLQTRNYGKVYVAGQGTSDSELVKKKGDIIL
TSLLGDGDHTLNVNKAESKELELYARVYNNTKRDITVDSVSLSPGLNAT
GREFSANKFVLYFKPTVLKKNRINTLVFGATFDEDIDDTNRHYLLSMRF
SPGNDLFKVGEK
SEQ ID 28:
VP28
>tr|A6ZI33|A6ZI33_WSSV Coat protein OS = White
spot syndrome virus OX = 92652 GN = VP28 PE = 4
SV = 1
MDLSFTLSVVSAILAITAVIAVFIVIFRYHNTVTKTIETHTGNIETNMD
ENLRIPVTAEVGSGYFKMTDVSFDSDTLGKIKIRNGKSDAQMKEEDADL
VITPVEGRALEVTVGQNLTFEGTFKMWNNTSRKINITGMQMVPKINPSK
AFVGSSNTSSFTPVSIDEDEVGTFVCGTTFGAPIAATAGGNLFDMYVHV
TYSGTETE

### EXAMPLES

The following illustrative examples are representative of the embodiments of the applications, systems and methods described herein and are not meant to be limiting in any way.

While preferred embodiments of the present invention are shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

### 1. Production of Antigens

Recombinant antigens can be purified from an E. coli expression system. For example, the gene for an antigen can be expressed at 18° C. in E. coli BL21 (DE3) cells grown overnight in autoinducing media (Formedium). Cells are then lysed by sonication in buffer A (250 mM NaCl, 50 mM CaCl₂, 20 mM Imidazole and 10 mM HEPES, pH 7.4) with 12.5 μg/ml DNase I, and 1× Protease inhibitor cocktail (Bioshop). The lysate is cleared by centrifugation at 22000×g for 30 minutes at 4° C., and is then applied to a 5 ml HisTrap HP column (GE Healthcare) pre-equilibrated with buffer A, washed with ten column volumes of buffer A and eluted with a gradient of 0% to 60% (vol/vol) buffer B (250 mM NaCl, 50 mM CaCl₂, 500 mM imidazole and 10 mM HEPES, pH 7.4). The protein is then dialyzed overnight in the presence of TEV against buffer C (250 mM NaCl, 10 mM HEPES, pH 7.4 and 5 mM β-mercaptoethanol) at 4° C. The dialyzed protein is applied to a HisTrap HP column (GE Biosciences) pre-equilibrated with buffer C. 6xHis-tagged TEV and 6xHis-tag are bound to the column and the antigen is collected in the flowthrough. The sample is dialyzed overnight against buffer D (5 mM NaCl and 10 mM Tris pH 8.8) and then applied to a 5 ml HiTrap Q HP column (GE Healthcare). The protein is eluted with a gradient of 0% to 50% (vol/vol) buffer E (1.0 M NaCl and 10 mM Tris pH 8.8). Lastly, the elution is loaded onto a Superdex 75 Increase 10/300 GL gel filtration column (GE Healthcare) using buffer F (400 mM NaCl and 20 mM HEPES pH 7.4). The protein sample is then concentrated to 1 mg/mL using Amicon concentrators with appropriate molecular weight cut-off (MWCO; Millipore). The purified protein is stored at −80° C.

### 2. Production of NBXs and Panning

### Llama Immunization

A single llama is immunized with purified disease-causing agents, such as the antigens listed, which may be accompanied by adjuvants. The llama immunization is performed using 100 μg of each antigen that are pooled and injected for a total of four injections. At the time of injection, the antigens are thawed, and the volume increased to 1 ml with PBS. The 1 ml antigen-PBS mixture is then mixed with 1 ml of Complete Freund's adjuvant (CFA) or Incomplete Freund's adjuvant (IFA) for a total of 2 ml. A total of 2 ml is immunized per injection. Whole llama blood and sera are then collected from the immunized animal on days 0, 28, 49, 70. Sera from days 28, 49 and 70 are then fractionated to separate V_{H}H from conventional antibodies. ELISA can be used to measure reactivity against target antigens in polyclonal and V_{H}H-enriched fractions. Lymphocytes are collected from sera taken at days 28, 49, and 70.

### Panning

RNA isolated from purified llama lymphocytes is used to generate cDNA for cloning into phagemids. The resulting phagemids are used to transform E. coli TG-1 cells to generate a library of expressed V_{H}H genes. The phagemid library size can be ˜2.5×10⁷ total transformants and the estimated number of phagemid containing V_{H}H inserts can be estimated to be ˜100%. High affinity antibodies are then selected by panning against the Vibrio or WSSV antigens used for llama immunization. At least two rounds of panning are performed and antigen-binding clones arising from rounds 2 or later are identified using phage ELISA. Antigen-binding clones are sequenced, grouped according to their CDR regions, and prioritized for soluble expression in E. coli and antibody purification.

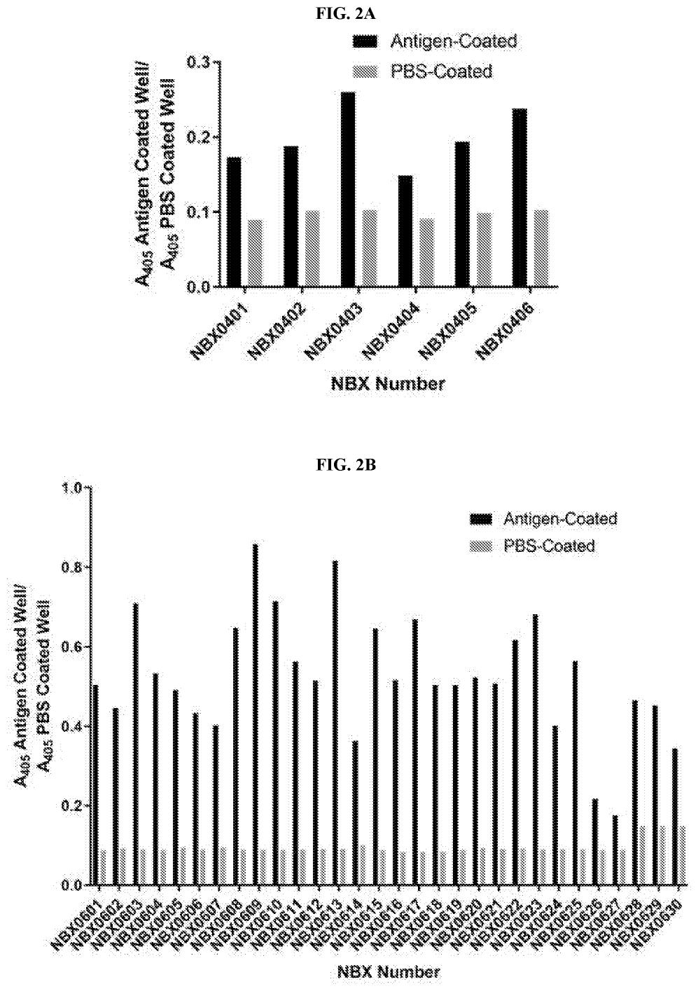

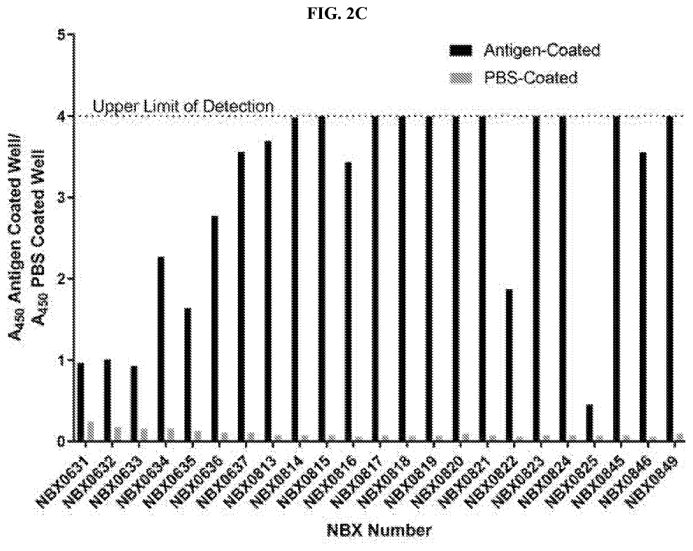

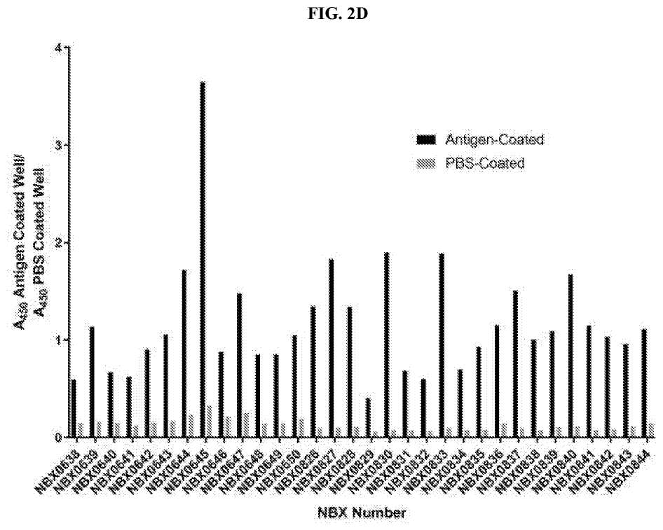

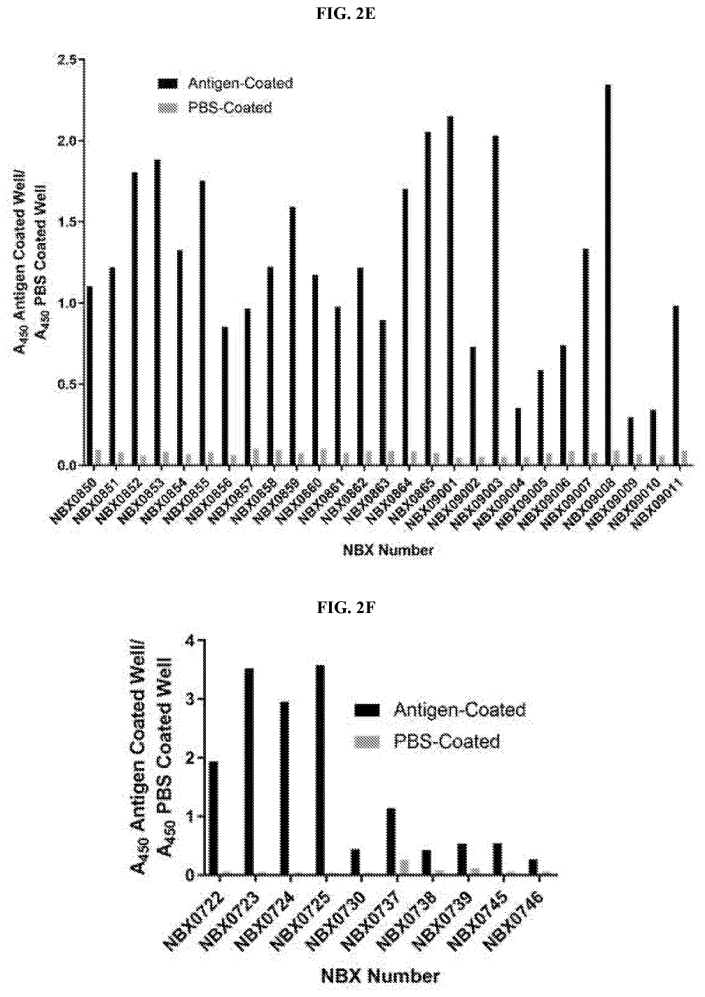

FIG. 2 shows the Phage ELISA results for all antibodies of this disclosure. Black bars show binding to wells coated with the antigen specified in Tables 1 and 2 dissolved in phosphate-buffered saline (PBS). Grey bars are negative controls that show binding to wells coated with PBS only. In all cases binding to the antigen target is at least 50% above binding to the PBS-coated wells. Panel A shows the results for NBX0401 to NBX0406. Panel B shows the results for NBX0601 to NBX0630. Panel C shows the results for NBX0631 to NBX0637, NBX0813 to NBX0825, NBX0845, NBX0846, and NBX0849. Panel D shows the results for NBX0638 to NBX0650, and NBX0826 to NBX0844. Panel E shows the results for NBX0850 to NBX0865, and NBX09001 to NBX09011. Panel F shows the results for NBX0722 to NBX0725, NBX0730, NBX0738, NBX0739, NBX0745, and NBX0746.

Purification of V_{H}Hs from E. coli

TEV protease-cleavable, 6xHis-thioredoxin-NBX fusion proteins are expressed in the cytoplasm of E. coli grown in autoinducing media (Formedium) for 24 hours at 30° C. Bacteria are collected by centrifugation, resuspended in buffer A (10 mM HEPES, pH 7.5, 250 mM NaCl, 20 mM Imidazole) and lysed using sonication. Insoluble material is removed by centrifugation and the remaining soluble fraction is applied to a HisTrap column (GE Biosciences) pre-equilibrated with buffer A. The protein is eluted from the column using an FPLC with a linear gradient between buffer A and buffer B (10 mM HEPES, pH 7.5, 500 mM NaCl, 500 mM Imidazole). The eluted protein is dialyzed overnight in the presence of TEV protease to buffer C (10 mM HEPES, pH 7.5, 500 mM NaCl). The dialyzed protein is applied to a HisTrap column (GE Biosciences) pre-equilibrated with buffer C. 6xHis-tagged TEV and 6xHis-tagged thioredoxin are bound to the column and highly purified NBX is collected in the flowthrough. NBX proteins are dialyzed overnight to PBS and concentrated to ˜10 mg/ml.

Purification of V_{H}Hs from P. pastoris

Pichia pastoris strain GS115 with constructs for the expression and secretion of 6xHis-tagged V_{H}H are grown for 5 days at 30° C. with daily induction of 0.5% (vol/vol) methanol. Yeast cells are removed by centrifugation and the NBX-containing supernatant is spiked with 10 mM imidazole. The supernatant is applied to a HisTrap column (GE Biosciences) pre-equilibrated with buffer A (10 mM HEPES, pH 7.5, 500 mM NaCl). The protein is eluted from the column using an FPLC with a linear gradient between buffer A and buffer B (10 mM HEPES, pH 7.5, 500 mM NaCl, 500 mM Imidazole). NBX proteins are dialyzed overnight to PBS and concentrated to ˜1.5 mg/ml.

### 3. Protein Pull-Downs

Approximately 0.1 mg of antigen is incubated with NBX at a 1:5 molar ratio in 200 μl of binding buffer (10 mM phosphate buffer pH7.4 and 500 mM NaCl) for 30 minutes at room temperature, and then applied onto a column containing Ni-NTA (nickel-nitrilotriacetic acid) resin pre-equilibrated with the binding buffer. Protein mixture and the resin are incubated for 30 minutes before the resin is washed with the binding buffer and then with the binding buffer plus 20 mM Imidazole. Bound proteins are eluted with 100 μl of 1 M imidazole, pH 7.4. The presence or absence of NBX in the various fractions is analyzed on an SDS-PAGE gel. A protein solution containing only the NBX is also applied to a separate column to assess non-specific binding of the NBX to the resin.

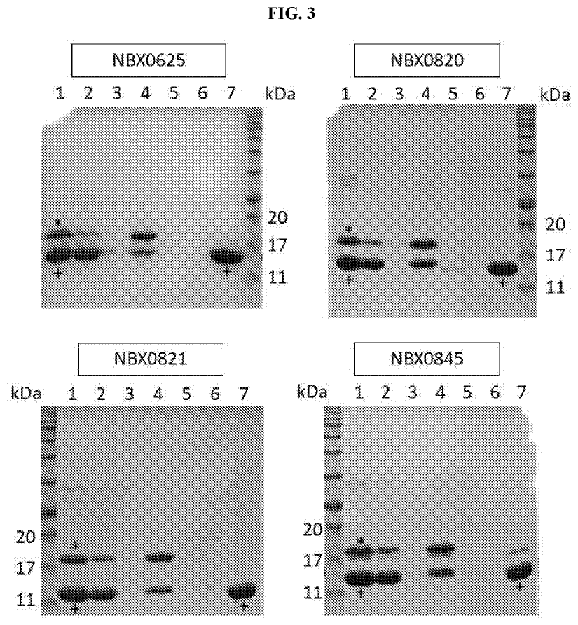

FIG. 3 shows representative results for four unique NBXs. For each of the four antibodies shown, the lanes are as follows. (1) Starting material of PirA(*) and NBX(⁺) mixture prior to application to Ni-NTA resin. (2) Flow-through of PirA and NBX through the Ni-NTA resin. (3) Final wash of the Ni-NTA resin prior to protein elution. (4) Elution of PirA and NBX from the Ni-NTA resin. (5) Elution from Ni-NTA resin to which only NBX was applied. (6) Final wash of Ni-NTA resin to which only NBX was applied. (7) NBX(⁺) only mixture prior to application to Ni-NTA resin. NBXs that can successfully be pulled down by PirA are those that appear in the lane 4 elution but not in the lane 5 elution. For each gel a ladder of proteins of known sizes in kilodaltons (kDa) are shown for reference.

### 4. Protein Stability in Shrimp Intestinal Tract Fluid

Thaw frozen shrimp midgut extract and NBX at room temperature, and immediately place on ice. Spin shrimp midgut extract and protein at 10,000 RCF for 1 minute to pellet and remove any precipitation. Prechill PBS and saline on ice. Label and prechill 8×0.2 mL strip tubes on ice. Set up two reactions in volumes of 10 μlon ice. The first reaction contains no shrimp midgut extract and consists of 5 μg NBX in 3.2 μL PBS and 4.8 μL of 150 mM NaCl. The second reaction contains shrimp midgut extract and is generated using the following ratios: 2.4 μL shrimp midgut extract, 5 μg NBX in 0.8 μL PBS, and 4.8 μL of 150 mM NaCl. The tubes are incubated on ice for 5 minutes (corresponds to time=0 minutes in FIG. 1) followed by 26° C. for up to 24 hours. The final incubation temperature (26° C.) is the internal temperature of a shrimp. After incubation, add 8 μL of preheated 2×SDS sample buffer to stop the reaction. Boil at 95-100° C. for 5 minutes. The stability of each NBXs is assessed by the presence or absence of the NBX on an 18% SDS-PAGE gel.

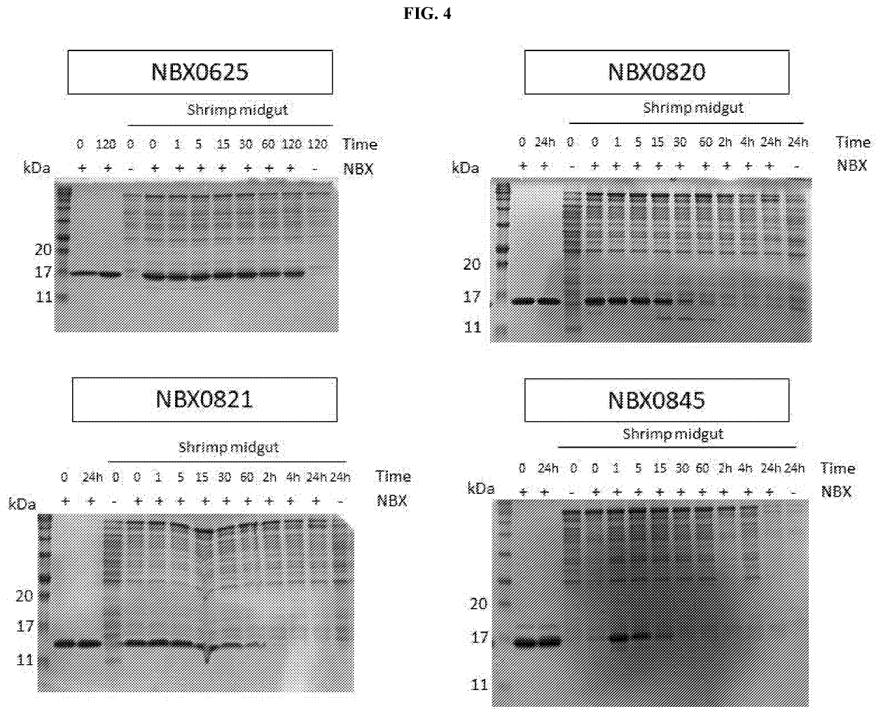

FIG. 4 shows representative results for four unique NBXs. For each of the four antibodies shown SDS-PAGE gels are arranged from left to right as follows. A ladder of proteins of known sizes in kilodaltons (kDa) are shown for reference. The next two lanes show the NBX at the beginning and end of the experiment in the absence of shrimp midgut extract. These lanes show that the NBX is not degraded over time in the absence of shrimp midgut extract. The subsequent lane shows the appearance of the shrimp midgut extract at the start of the experiment without NBX added. This lane allows for the visualization of naturally occurring proteins in the extract. The subsequent 7-9 lanes show the time course of NBX stability in the shrimp midgut extract. These lanes allow for the visualization of the relative stability of the NBX. The longer the full-sized NBX can be visualized on the gel the more stable it is. The final lane shows the shrimp midgut extract in the absence of NBX at the endpoint of the assay.

All publications, patent applications, issued patents, and other documents referred to in this specification are herein incorporated by reference as if each individual publication, patent application, issued patent, or other document is specifically and individually indicated to be incorporated by reference in its entirety. Definitions that are contained in text incorporated by reference are excluded to the extent that they contradict definitions in this disclosure.

The following references are incorporated by reference in their entirety.
1. Kierath, D. (2015, March). The growth of global aquaculture—Fishy business. Retrieved from deloitte.com/au/en/pages/consumer-business/articles/the-growth-of-aqua-culture-fishy-business
2. Stentiford, G. D., Sritunyalucksana, K., Flegel, T. W., Williams, B. A. P., Withyachumnarnkul, B., Itathitphaisarn, O., Bass, D. (2017). New paradigms to help solve the global aquaculture disease crisis. PLos Pathogens, 13(2), pp. 1-6
3. Lee, C. T., Chen, T. I., Yang, Y. T., Ko, T. P., Huang, Y. T., Huang., J. Y., Huang, M. F., Lin, S. J., Chen, C. Y., Lin, S. S., Lightner, D. V., Wang, H. C., Wang, A. H. J., Wang, H. C., Hor, L. I., Lo, C. F. (2015) The opportunistic marine pathogen Vibrio parahaemolyticus becomes virulent by acquiring a plasmid that expresses a deadly toxin. PNAS, 112(34), pp. 10789-10803.
4. FAO Fisheries and Aquaculture (2013) Report of the FAO/MARD Technical Workshop on Early Mortality Syndrome (EMS) or Acute Hepatopancreatic Necrosis Syndrome (AHPNS) of Cultured Shrimp (under TCP/VIE/3304). rep. no. 1053. Retrieved from www.fao.org/docrep/018/i3422e/i3422e.pdf
5. Tran, L., Numan, L., Redman, R. M., Mohney, L. L., Pantoj a, C. R., Fitzsimmons, K., Lightner, D. V. (2013) Determination of the infectious nature of the agent of acute hepatopancreatic necrosis syndrome affecting penaeid shrimp. Diseases of Aquatic Organisms, 105(1), pp. 45-55.
6. Ahmed, H. A., El Bayomi, R. M., Hussein, M. A., Khedr, M. H. E., Abo Ramela, E. M., El-Ashram, A. M. M. (2018) Molecular characterization, antibiotic resistance pattern and biofilm formation of Vibrio parahaemolyticus and V. cholerae isolated from crustaceans and humans. International Journal of Food Microbiology, 274, pp. 31-37.
7. Flegel, T. (2012) Historic emergence, impact and current status of shrimp pathogens in Asia, Journal of Invertebrate Pathology, 110(2), pp. 166-173.
8. Lightner, D. V., Redman, R. M., Pantoja, C. R., Tang, K. F. J., Noble, B. L., Schofield, P., Mohney, L. L., Nunan, L. M., Navarro, S. A. (2012) Historic emergence, impact and current status of shrimp pathogens in the Americas, Journal of Invertebrate Pathology, 110 (2), pp. 174-183.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A polypeptide comprising at least one variable region fragment of a heavy chain antibody (V_{H}H), wherein the at least one V_{H}H specifically binds a species of Vibrio or a White Spot Syndrome virus.

2. (canceled)

3. The polypeptide of claim 1, wherein the V_{H}H comprises an amino acid sequence with at least 80% identity to the amino acid sequence set forth in any one of SEQ ID Nos: 1 or 2 or 4 or 5 or 53 or 96 or 97 or 121.

4. The polypeptide of claim 1, wherein the polypeptide comprises a plurality of V_{H}Hs.

5. The polypeptide of claim 4, wherein the polypeptide comprises at least three V_{H}Hs.

6. The polypeptide of claim 4, wherein any one of the plurality of V_{H}Hs is identical to another V_{H}H of the plurality of V_{H}Hs.

7. The polypeptide of claim 4, wherein the plurality of V_{H}Hs are covalently coupled to one another by a linker, the linker comprising one or more amino acids.

8. The polypeptide of claim 1, wherein the at least one variable region fragment of the heavy chain antibody comprises an amino acid sequence at least 80%, 90%, 95%, 97%, 98%, 99%, or 100% identical to any one of SEQ ID Nos: 1 to 6 or 29 to 150.

9. (canceled)

10. The polypeptide of claim 1, wherein the amino acid sequence of the V_{H}H comprises:
(a) a CDR1 sequence set forth in SEQ ID No: 7, a CDR2 sequence set forth in SEQ ID No: 13, and a CDR3 sequence set forth in SEQ ID No: 19.
(b) a CDR1 sequence set forth in SEQ ID No: 8, a CDR2 sequence set forth in SEQ ID No: 14, and a CDR3 sequence set forth in SEQ ID No: 20.
(c) a CDR1 sequence set forth in SEQ ID No: 10, a CDR2 sequence set forth in SEQ ID No: 16, and a CDR3 sequence set forth in SEQ ID No: 22.
(d) a CDR1 sequence set forth in SEQ ID No: 11, a CDR2 sequence set forth in SEQ ID No: 17, and a CDR3 sequence set forth in SEQ ID No: 23.
(e) a CDR1 sequence set forth in SEQ ID No: 175, a CDR2 sequence set forth in SEQ ID No: 297, and a CDR3 sequence set forth in SEQ ID No: 419.
(f) a CDR1 sequence set forth in SEQ ID No: 218, a CDR2 sequence set forth in SEQ ID No: 340, and a CDR3 sequence set forth in SEQ ID No: 462.
(g) a CDR1 sequence set forth in SEQ ID No: 219, a CDR2 sequence set forth in SEQ ID No: 341, and a CDR3 sequence set forth in SEQ ID No: 463.
(h) a CDR1 sequence set forth in SEQ ID No: 243, a CDR2 sequence set forth in SEQ ID No: 365, and a CDR3 sequence set forth in SEQ ID No: 487.

11. (canceled)

12. The polypeptide of claim 1, wherein the at least one V_{H}H that specifically binds a species of Vibrio or a White Spot Syndrome virus binds a White Spot Syndrome virus.

13. The polypeptide of claim 1, wherein the at least one V_{H}H that specifically binds a species of Vibrio or a White Spot Syndrome virus binds a species of Vibrio.

14. The polypeptide of claim 1, wherein the species of Vibrio is selected from the list consisting of V. adaptatus, V. aerogenes, V. aestivus, V. aestuarianus, V. agarivorans, V. albensis, V. alfacsensis, V. alginolyticus, V. anguillarum, V. areninigrae, V. artabrorum, V. atlanticus, V. atypicus, V. azureus, V. brasiliensis, V. bubulus, V. calviensis, V. campbellii, V. casei, V. chagasii, V. cholerae, V. cincinnatiensis, V. coralliilyticus, V. crassostreae, V. cyclitrophicus, V. diabolicus, V. diazotrophicus, V. ezurae, V. fluvialis, V. fortis, V. furnissii, V. gallicus, V. gazogenes, V. gigantis, V. halioticoli, V. harveyi, V. hepatarius, V. hippocampi, V. hispanicus, V. ichthyoenteri, V. indicus, V. kanaloae, V. lentus, V. litoralis, V. logei, V. mediterranei, V. metschnikovii, V. mimicus, V. mytili, V. natriegens, V. navarrensis, V. neonatus, V. neptunius, V. nereis, V. nignpulchritudo, V. ordalii, V. orientalis, V. pacinii, V. parahaemolyticus, V. pectenicida, V. penaeicida, V. pomeroyi, V. ponticus, V. proteolyticus, V. rotiferianus, V. ruber, V. rumoiensis, V. salmonicida, V. scophthalmi, V. splendidus, V. superstes, V. tapetis, V. tasmaniensis, V. tubiashii, V. vulnificus, V. wodanis, V. xuii, V. fischer, and V. hollisae.

15. (canceled)

16. The polypeptide of claim 1, wherein the V_{H}H specifically binds an antigen or polypeptide at least 60%, 70%, 80%, 90%, 95%, 98%, 99%, or 100% identical to SEQ IDs Nos: 25 or 26 or combinations thereof.

17. (canceled)

18. (canceled)

19. The polypeptide of claim 1, wherein the V_{H}H specifically binds an antigen or polypeptide at least 60%, 70%, 80%, 90%, 95%, 98%, 99%, or 100% identical to SEQ IDs Nos: 27 or 28 or combinations thereof.

20. (canceled)

21. The polypeptide of claim 1, wherein the V_{H}H can be pulled-down in a protein-protein binding assay by any of SEQ ID Nos: 25 or 26 or 27 or 28.

22. The polypeptide of claim 1, wherein the V_{H}H survives in shrimp midgut extract for at least 1 minute, 5 minutes, 15 minutes, 30 minutes, 1 hour, 2 hours, 4 hours, or 24 hours.

23. A nucleic acid or a plurality of nucleic acids encoding the polypeptide of claim 1.

24. (canceled)

25. A cell comprising the nucleic acid or the plurality of nucleic acids of claim 23.

26.-33. (canceled)

34. A method of producing the polypeptide of claim 1, comprising (a) incubating a cell comprising nucleic acids encoding the polypeptide in a medium suitable for secretion of the polypeptide from the cell; and (b) purifying the polypeptide from the medium.

35.-39. (canceled)

40. A method of reducing transmission or preventing transmission of a species of Vibrio or a White Spot Syndrome virus from a fish or shellfish to another fish or shellfish comprising administering the polypeptide of claim 1 to an aquaculture comprising the fish or the shellfish, thereby reducing transmission or preventing transmission of a species of Vibrio or a White Spot Syndrome virus.

41. A method of reducing, treating, or preventing an infection by a species of Vibrio or a White Spot Syndrome virus in a human individual comprising administering the polypeptide of claim 1 to the human individual, thereby reducing, treating, or preventing infection by the species of Vibrio or the White Spot Syndrome virus.
